SEQUENCE LISTING (1510 sequences)

SEQ ID NO: 1 - DNA, 20 bp, synthetic construct
  misc_feature 1..20: note = Synthetic
  source 1..20: mol_type = other DNA
tagcactgaagggttctgaa

SEQ ID NO: 2 - DNA, 20 bp, synthetic construct
  misc_feature 1..20: note = Synthetic
  source 1..20: mol_type = other DNA
tgtgaggggttaaggctggg

SEQ ID NO: 3 - DNA, 20 bp, synthetic construct
  misc_feature 1..20: note = Synthetic
  source 1..20: mol_type = other DNA
tctctggccacttctctctg

SEQ ID NO: 4 - DNA, 20 bp, synthetic construct
  misc_feature 1..20: note = Synthetic
  source 1..20: mol_type = other DNA
gacaggcagacatgcagcca

SEQ ID NO: 5 - DNA, 20 bp, synthetic construct
  misc_feature 1..20: note = Synthetic
  source 1..20: mol_type = other DNA
atgttggacaggcagcacca

SEQ ID NO: 6 - DNA, 20 bp, synthetic construct
  misc_feature 1..20: note = Synthetic
  source 1..20: mol_type = other DNA
ccaacatgtcagccgaggtg

SEQ ID NO: 7 - DNA, 20 bp, synthetic construct
  misc_feature 1..20: note = Synthetic
  source 1..20: mol_type = other DNA
catggcttaggagtcacagc

SEQ ID NO: 8 - DNA, 20 bp, synthetic construct
  misc_feature 1..20: note = Synthetic
  source 1..20: mol_type = other DNA
tgaccctgaccctgaccctg

SEQ ID NO: 9 - DNA, 20 bp, synthetic construct
  misc_feature 1..20: note = Synthetic
  source 1..20: mol_type = other DNA
cccaggccacaggaaaccag

SEQ ID NO: 10 - DNA, 20 bp, synthetic construct
  misc_feature 1..20: note = Synthetic
  source 1..20: mol_type = other DNA
ttgtccctcctggatcgcag

SEQ ID NO: 11 - DNA, 20 bp, synthetic construct
  misc_feature 1..20: note = Synthetic
  source 1..20: mol_type = other DNA
ggaatggtggctgacccaca

SEQ ID NO: 12 - DNA, 20 bp, synthetic construct
  misc_feature 1..20: note = Synthetic
  source 1..20: mol_type = other DNA
aaccaaacaccagtcactga

SEQ ID NO: 13 - DNA, 20 bp, synthetic construct
  misc_feature 1..20: note = Synthetic
  source 1..20: mol_type = other DNA
catcacacctgtggcttcct

SEQ ID NO: 14 - DNA, 20 bp, synthetic construct
  misc_feature 1..20: note = Synthetic
  source 1..20: mol_type = other DNA
taccctgccaacccaacttc

SEQ ID NO: 15 - DNA, 21 bp, synthetic construct
  misc_feature 1..21: note = Synthetic
  source 1..21: mol_type = other DNA
gagcaacaccttcacggtcaa

SEQ ID NO: 16 - DNA, 22 bp, synthetic construct
  misc_feature 1..22: note = Synthetic
  source 1..22: mol_type = other DNA
cgcgttgtcactgttcttcttt

SEQ ID NO: 17 - DNA, 19 bp, synthetic construct
  misc_feature 1..19: note = Synthetic
  source 1..19: mol_type = other DNA
taaactgttttcgcggctg

SEQ ID NO: 18 - DNA, 26 bp, synthetic construct
  misc_feature 1..26: note = Synthetic
  source 1..26: mol_type = other DNA
tccctagttcaagaggaagaaatacc

SEQ ID NO: 19 - DNA, 23 bp, synthetic construct
  misc_feature 1..23: note = Synthetic
  source 1..23: mol_type = other DNA
tgccatgtggaaaagacttccta

SEQ ID NO: 20 - DNA, 16 bp, synthetic construct
  misc_feature 1..16: note = Synthetic
  source 1..16: mol_type = other DNA
ctggaaagccaatgag

SEQ ID NO: 21 - DNA, 23 bp, synthetic construct
  misc_feature 1..23: note = Synthetic
  source 1..23: mol_type = other DNA
tgatgaggatggtgatgatatgg

SEQ ID NO: 22 - DNA, 22 bp, synthetic construct
  misc_feature 1..22: note = Synthetic
  source 1..22: mol_type = other DNA
atctgaccacctctcccttacg

SEQ ID NO: 23 - DNA, 22 bp, synthetic construct
  misc_feature 1..22: note = Synthetic
  source 1..22: mol_type = other DNA
tagttatcaggaagaagactta

SEQ ID NO: 24 - protein, 7 aa, synthetic construct
  REGION 1..7: note = Synthetic
PKKKRKV

SEQ ID NO: 25 - protein, 16 aa, synthetic construct
  REGION 1..16: note = Synthetic
KRPAATKKAGQAKKKK

SEQ ID NO: 26 - DNA, 19 bp, synthetic construct
  misc_feature 1..19: note = Synthetic
  source 1..19: mol_type = other DNA
aactaaaggacgcagggac

SEQ ID NO: 27 - DNA, 19 bp, synthetic construct
  misc_feature 1..19: note = Synthetic
  source 1..19: mol_type = other DNA
tcctatgccaccactctgg

SEQ ID NO: 28-100 - skipped (73 entries)

SEQ ID NO: 101 - DNA, 19 bp, synthetic construct
  misc_feature 1..19: note = Synthetic
  source 1..19: mol_type = other DNA
tagcactgaagggttctga

SEQ ID NO: 102 - DNA, 18 bp, synthetic construct
  misc_feature 1..18: note = Synthetic
  source 1..18: mol_type = other DNA
tagcactgaagggttctg

SEQ ID NO: 103 - DNA, 17 bp, synthetic construct
  misc_feature 1..17: note = Synthetic
  source 1..17: mol_type = other DNA
tagcactgaagggttct

SEQ ID NO: 104-200 - skipped (97 entries)

SEQ ID NO: 201 - DNA, 19 bp, synthetic construct
  misc_feature 1..19: note = Synthetic
  source 1..19: mol_type = other DNA
tgtgaggggttaaggctgg

SEQ ID NO: 202 - DNA, 18 bp, synthetic construct
  misc_feature 1..18: note = Synthetic
  source 1..18: mol_type = other DNA
tgtgaggggttaaggctg

SEQ ID NO: 203 - DNA, 17 bp, synthetic construct
  misc_feature 1..17: note = Synthetic
  source 1..17: mol_type = other DNA
tgtgaggggttaaggct

SEQ ID NO: 204-300 - skipped (97 entries)

SEQ ID NO: 301 - DNA, 19 bp, synthetic construct
  misc_feature 1..19: note = Synthetic
  source 1..19: mol_type = other DNA
ctctggccacttctctctg

SEQ ID NO: 302 - DNA, 18 bp, synthetic construct
  misc_feature 1..18: note = Synthetic
  source 1..18: mol_type = other DNA
tctggccacttctctctg

SEQ ID NO: 303 - DNA, 17 bp, synthetic construct
  misc_feature 1..17: note = Synthetic
  source 1..17: mol_type = other DNA
ctggccacttctctctg

SEQ ID NO: 304-400 - skipped (97 entries)

SEQ ID NO: 401 - DNA, 19 bp, synthetic construct
  misc_feature 1..19: note = Synthetic
  source 1..19: mol_type = other DNA
gacaggcagacatgcagcc

SEQ ID NO: 402 - DNA, 18 bp, synthetic construct
  misc_feature 1..18: note = Synthetic
  source 1..18: mol_type = other DNA
gacaggcagacatgcagc

SEQ ID NO: 403 - DNA, 17 bp, synthetic construct
  misc_feature 1..17: note = Synthetic
  source 1..17: mol_type = other DNA
gacaggcagacatgcag

SEQ ID NO: 404-500 - skipped (97 entries)

SEQ ID NO: 501 - DNA, 19 bp, synthetic construct
  misc_feature 1..19: note = Synthetic
  source 1..19: mol_type = other DNA
tgttggacaggcagcacca

SEQ ID NO: 502 - DNA, 18 bp, synthetic construct
  misc_feature 1..18: note = Synthetic
  source 1..18: mol_type = other DNA
gttggacaggcagcacca

SEQ ID NO: 503 - DNA, 17 bp, synthetic construct
  misc_feature 1..17: note = Synthetic
  source 1..17: mol_type = other DNA
ttggacaggcagcacca

SEQ ID NO: 504-600 - skipped (97 entries)

SEQ ID NO: 601 - DNA, 19 bp, synthetic construct
  misc_feature 1..19: note = Synthetic
  source 1..19: mol_type = other DNA
ccaacatgtcagccgaggt

SEQ ID NO: 602 - DNA, 18 bp, synthetic construct
  misc_feature 1..18: note = Synthetic
  source 1..18: mol_type = other DNA
ccaacatgtcagccgagg

SEQ ID NO: 603 - DNA, 17 bp, synthetic construct
  misc_feature 1..17: note = Synthetic
  source 1..17: mol_type = other DNA
ccaacatgtcagccgag

SEQ ID NO: 604-700 - skipped (97 entries)

SEQ ID NO: 701 - DNA, 19 bp, synthetic construct
  misc_feature 1..19: note = Synthetic
  source 1..19: mol_type = other DNA
catggcttaggagtcacag

SEQ ID NO: 702 - DNA, 18 bp, synthetic construct
  misc_feature 1..18: note = Synthetic
  source 1..18: mol_type = other DNA
catggcttaggagtcaca

SEQ ID NO: 703 - DNA, 17 bp, synthetic construct
  misc_feature 1..17: note = Synthetic
  source 1..17: mol_type = other DNA
catggcttaggagtcac

SEQ ID NO: 704-709 - skipped (6 entries)

SEQ ID NO: 710 - protein, 1368 aa, Streptococcus pyogenes [sequence omitted]

SEQ ID NO: 711 - protein, 1052 aa, Staphylococcus aureus
  REGION 1..1052: note = misc_feature - Staphylococcus aureus Cas9
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 712 - protein, 1053 aa, Staphylococcus lugdunensis
  REGION 1..1053: note = misc_feature - Staphylococcus lugdunensis Cas9
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 713-714 - skipped (2 entries)

SEQ ID NO: 715 - protein, 1052 aa, synthetic construct
  REGION 1..1052: note = Synthetic: designated herein as SaCas9-KKH or SACAS9KKH
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 716 - protein, 1052 aa, synthetic construct
  REGION 1..1052: note = Synthetic: designated herein as SaCas9-HF
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELASVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDAQIAIFARLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 717 - protein, 1052 aa, synthetic construct
  REGION 1..1052: note = Synthetic: SaCas9-KKH-HF
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELASVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDAQIAIFARLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATAGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 718 - protein, 1053 aa, synthetic construct
  REGION 1..1053: note = Synthetic: SluCas9-KH or SLUCAS9KH
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRKLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPHIKKTIGKKVNSIEKLTTDVLGNVFTNTQYTKPQLLFKRGN

SEQ ID NO: 719 - protein, 1053 aa, synthetic construct
  REGION 1..1053: note = Synthetic: SluCas9-HF
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELASVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRAQMEIFAHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATAELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 720 - protein, 1053 aa, synthetic construct
  REGION 1..1053: note = Synthetic: SluCas9-HF-KH
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELASVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRAQMEIFAHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATAELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRKLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPHIKKTIGKKVNSIEKLTTDVLGNVFTNTQYTKPQLLFKRGN

SEQ ID NO: 721 - protein, 1057 aa, synthetic construct
  REGION 1..1057: note = Synthetic: sRGN1
MNQKFILGLDIGITSVGYGLIDYETKNIIDAGVRLFPEANVENNEGRRSKRGSRRLKRRRIHRLDRVKHLLAEYDLLDLTNIPKSTNPYQTRVKGLNEKLSKDELVIALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGTPEFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 722 - protein, 1053 aa, synthetic construct
  REGION 1..1053: note = Synthetic: sRGN2
MNQKFILGLDIGITSVGYGLIDYETKNIIDAGVRLFPEANVENNEGRRSKRGSRRLKRRRIHRLERVKSLLSEYKIISGLAPTNNQPYNIRVKGLTEQLTKDELAVALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTEFKLYHDLKSVLFDQSILENEDVLDQIAEILTIYQDKDSIKSKLTELDILLNEEDKENIAQLTGYNGTHRLSLKCIRLVLEEQWYSSRNQMEIFTHLNIKPKKINLTAANKIPKAMIDEFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 723 - protein, 1057 aa, synthetic construct
  REGION 1..1057: note = Synthetic: sRGN3
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 724 - protein, 1057 aa, synthetic construct
  REGION 1..1057: note = Synthetic: sRGN3.1
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 725 - protein, 1057 aa, synthetic construct
  REGION 1..1057: note = Synthetic: sRGN3.2
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 726 - protein, 1057 aa, synthetic construct
  REGION 1..1057: note = Synthetic: sRGN3.3
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 727 - protein, 1054 aa, synthetic construct
  REGION 1..1054: note = Synthetic: sRGN4
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 728 - protein, 1051 aa, Staphylococcus hyicus
  REGION 1..1051: note = misc_feature - Staphylococcus hyicus Cas9 or ShyCas9
MNNYILGLDIGITSVGYGIVDSDTREIKDAGVRLFPEANVDNNEGRRSKRGARRLKRRRIHRLDRVKHLLAEYDLLDLTNIPKSTNPYQTRVKGLNEKLSKDELVIALLHIAKRRGIHNVNVMMDDNDSGNELSTKDQLKKNAKALSDKYVCELQLERFEQDYKVRGEKNRFKTEDFVREARKLLETQSKFFEIDQTFIMRYIELIETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGKPEFTQFKLYHDLKNIFKDPKYLNDIQLMDNIAEIITIYQDAESIIKELNQLPELLSEREKEKISALSGYSGTHRLSLKCINLLLDDLWESSLNQMELFTKLNLKPKKIDLSQQHKIPSKLVDDFILSPVVKRAFIQSIQVVNAIIDKYGLPEDIIIELARENNSDDRRKFLNQLQKQNEETRKQVEKVLREYGNDNAKRIVQKIKLHNMQEGKCLYSLKDIPLEDLLRNPHHYEVDHIIPRSVAFDNSMHNKVLVRADENSKKGNRTPYQYLNSSESSLSYNEFKQHILNLSKTKDRITKKKREYLLEERDINKFDVQKEFINRNLVDTRYATRELTSLLKAYFSANNLDVKVKTINGSFTNYLRKVWKFDKDRNKGYKHHAEDALIIANADFLFKHNKKLRNINKVLDAPSKEVDKKRVTVQSEDEYNQIFEDTQKAQAIKKFEIRKFSHRVDKKPNRQLINDTLYSTRNIDGIEYVVESIKDIYSVNNDKVKTKFKKDPHRLLMYRNDPQTFEKFEKVFKQYESEKNPFAKYYEETGEKIRKFSKTGQGPYINKIKYLRERLGRHCDVTNKYINSRNKIVQLKIYSYRFDIYQYGNNYKMITISYIDLEQKSNYYYISREKYEQKKKDKQIDDSYKFIGSFYKNDIINYNGEMYRVIGVNDSEKNKIQLDMIDISIKDYMELNNIKKTGVIYKTIGKSTTHIEKYTTDILGNLYKAAPPKKPQLIFK

SEQ ID NO: 729 - protein, 1052 aa, Staphylococcus microti
  REGION 1..1052: note = misc_feature - Staphylococcus microti Cas9 or Smi Cas9
MEKDYILGLDIGIGSVGYGLIDYDTKSIIDAGVRLFPEANADNNLGRRAKRGARRLKRRRIHRLERVKSLLSEYKIISGLAPTNNQPYNIRVKGLTEQLTKDELAVALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYYEGPGKGSPYGWDADVKKWYQLMMGHCTYFPVEFRSVKYAYTADLYNALNDLNNLTIARDDNPKLEYHEKYHIIENVFKQKRNPTLKQIAKEIGVNDINISGYRVTKSGKPQFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRSFKQAIGVVNAIIKKYGLPKDIIIELARESNSAEKSRYLRAIQKKNEKTRERIEAIIKEYGNENAKGLVQKIKLHDAQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERNINKYDVQKEFINRNLVDTRYTTRELTTLLKTYFTINNLDVKVKTINGSFTDFLRKRWGFKKNRDEGYKHHAEDALIIANADYLFKEHKLLKEIKDVSDLAGDERNSNVKDEDQYEEVFGGYFKIEDIKKYKIKKFSHRVDKKPNRQLINDTIYSTRVKDDKRYLINTLKNLYDKSNGDLKERMQKDPESLLMYHHDPQTFEKLKIVMSQYENEKNPLAKYFEETGQYLTKYAKHDNGPAIHKIKYYGNKLVEHLDITKNYHNPQNKVVQLSQKSFRFDVYQTDKGYKFISIAYLTLKNEKNYYAISQEKYDQLKSEKKISNNAVFIGSFYTSDIIEINNEKFRVIGVNSDKNNLIEVDRIDIRQKEFIELEEEKKNNRIKVTIGRKTTNIEKFHTDILGNMYKSKRPKAPQLVFKKG

SEQ ID NO: 730 - protein, 1054 aa, Staphylococcus pasteuri
  REGION 1..1054: note = misc_feature - Staphylococcus pasteuri Cas9 or Spa Cas9
MKEKYILGLDLGITSVGYGIINFETKKIIDAGVRLFPEANVDNNEGRRSKRGSRRLKRRRIHRLERVKLLLTEYDLINKEQIPTSNNPYQIRVKGLSEILSKDELAIALLHLAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPQFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 731 - protein, 1093 aa, synthetic construct
  REGION 1..1093: note = Synthetic: Cas12i1
MSNKEKNASETRKAYTTKMIPRSHDRMKLLGNFMDYLMDGTPIFFELWNQFGGGIDRDIISGTANKDKISDDLLLAVNWFKVMPINSKPQGVSPSNLANLFQQYSGSEPDIQAQEYFASNFDTEKHQWKDMRVEYERLLAELQLSRSDMHHDLKLMYKEKCIGLSLSTAHYITSVMFGTGAKNNRQTKHQFYSKVIQLLEESTQINSVEQLASIILKAGDCDSYRKLRIRCSRKGATPSILKIVQDYELGTNHDDEVNVPSLIANLKEKLGRFEYECEWKCMEKIKAFLASKVGPYYLGSYSAMLENALSPIKGMTTKNCKFVLKQIDAKNDIKYENEPFGKIVEGFFDSPYFESDTNVKWVLHPHHIGESNIKTLWEDLNAIHSKYEEDIASLSEDKKEKRIKVYQGDVCQTINTYCEEVGKEAKTPLVQLLRYLYSRKDDIAVDKIIDGITFLSKKHKVEKQKINPVIQKYPSFNFGNNSKLLGKIISPKDKLKHNLKCNRNQVDNYIWIEIKVLNTKTMRWEKHHYALSSTRFLEEVYYPATSENPPDALAARFRTKTNGYEGKPALSAEQIEQIRSAPVGLRKVKKRQMRLEAARQQNLLPRYTWGKDFNINICKRGNNFEVTLATKVKKKKEKNYKVVLGYDANIVRKNTYAAIEAHANGDGVIDYNDLPVKPIESGFVTVESQVRDKSYDQLSYNGVKLLYCKPHVESRRSFLEKYRNGTMKDNRGNNIQIDFMKDFEAIADDETSLYYFNMKYCKLLQSSIRNHSSQAKEYREEIFELLRDGKLSVLKLSSLSNLSFVMFKVAKSLIGTYFGHLLKKPKNSKSDVKAPPITDEDKQKADPEMFALRLALEEKRLNKVKSKKEVIANKIVAKALELRDKYGPVLIKGENISDTTKKGKKSSTNSFLMDWLARGVANKVKEMVMMHQGLEFVEVNPNFTSHQDPFVHKNPENTFRARYSRCTPSELTEKNRKEILSFLSDKPSKRPTNAYYNEGAMAFLATYGLKKNDVLGVSLEKFKQIMANILHQRSEDQLLFPSRGGMFYLATYKLDADATSVNWNGKQFWVCNADLVAAYNVGLVDIQKDFKKK

SEQ ID NO: 732 - protein, 1054 aa, synthetic construct
  REGION 1..1054: note = Synthetic: Cas12i2
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 733-800 - skipped (68 entries)

SEQ ID NO: 801 - DNA, 19 bp, synthetic construct
  misc_feature 1..19: note = Synthetic
  source 1..19: mol_type = other DNA
gaccctgaccctgaccctg

SEQ ID NO: 802 - DNA, 18 bp, synthetic construct
  misc_feature 1..18: note = Synthetic
  source 1..18: mol_type = other DNA
accctgaccctgaccctg

SEQ ID NO: 803 - DNA, 17 bp, synthetic construct
  misc_feature 1..17: note = Synthetic
  source 1..17: mol_type = other DNA
ccctgaccctgaccctg

SEQ ID NO: 804-900 - skipped (97 entries)

SEQ ID NO: 901 - DNA, 19 bp, synthetic construct
  misc_feature 1..19: note = Synthetic
  source 1..19: mol_type = other DNA
ccaggccacaggaaaccag

SEQ ID NO: 902 - DNA, 18 bp, synthetic construct
  misc_feature 1..18: note = Synthetic
  source 1..18: mol_type = other DNA
caggccacaggaaaccag

SEQ ID NO: 903 - DNA, 17 bp, synthetic construct
  misc_feature 1..17: note = Synthetic
  source 1..17: mol_type = other DNA
aggccacaggaaaccag

SEQ ID NO: 904-913 - skipped (10 entries)

SEQ ID NO: 914 - DNA, 3156 bp, synthetic construct
  misc_feature 1..3156: note = Synthetic
  source 1..3156: mol_type = other DNA
aagcgcaattacatcctgggcctggatatcggcatcacctccgtgggctacggcatcatcgactatgagacacgggatgtgatcgacgccggcgtgagactgttcaaggaggccaacgtggagaacaatgagggccggcggagcaagaggggagcaaggcgcctgaagcggagaaggcgccacagaatccagagagtgaagaagctgctgttcgattacaacctgctgaccgaccactccgagctgtctggcatcaatccttatgaggcccgggtgaagggcctgtcccagaagctgtctgaggaggagttttctgccgccctgctgcacctggcaaagaggagaggcgtgcacaacgtgaatgaggtggaggaggacaccggcaacgagctgagcacaaaggagcagatcagccgcaattccaaggccctggaggagaagtatgtggccgagctgcagctggagcggctgaagaaggatggcgaggtgaggggctccatcaatcgcttcaagacctctgactacgtgaaggaggccaagcagctgctgaaggtgcagaaggcctaccaccagctggatcagagctttatcgatacatatatcgacctgctggagaccaggcgcacatactatgagggaccaggagagggctcccccttcggctggaaggacatcaaggagtggtacgagatgctgatgggccactgcacctattttccagaggagctgagatccgtgaagtacgcctataacgccgatctgtacaacgccctgaatgacctgaacaacctggtcatcaccagggatgagaacgagaagctggagtactatgagaagttccagatcatcgagaacgtgttcaagcagaagaagaagcctacactgaagcagatcgccaaggagatcctggtgaacgaggaggacatcaagggctaccgcgtgaccagcacaggcaagccagagttcaccaatctgaaggtgtatcacgatatcaaggacatcacagcccggaaggagatcatcgagaacgccgagctgctggatcagatcgccaagatcctgaccatctatcagagctccgaggacatccaggaggagctgaccaacctgaatagcgagctgacacaggaggagatcgagcagatcagcaatctgaagggctacaccggcacacacaacctgtccctgaaggccatcaatctgatcctggatgagctgtggcacacaaacgacaatcagatcgccatctttaacaggctgaagctggtgccaaagaaggtggacctgagccagcagaaggagatcccaaccacactggtggacgatttcatcctgtcccccgtggtgaagcggagcttcatccagagcatcaaagtgatcaacgccatcatcaagaagtacggcctgcccaatgatatcatcatcgagctggccagggagaagaactctaaggacgcccagaagatgatcaatgagatgcagaagaggaaccgccagaccaatgagcggatcgaggagatcatcagaaccacaggcaaggagaacgccaagtacctgatcgagaagatcaagctgcacgatatgcaggagggcaagtgtctgtatagcctggaggccatccctctggaggacctgctgaacaatccattcaactacgaggtggatcacatcatcccccggagcgtgagcttcgacaattcctttaacaataaggtgctggtgaagcaggaggagaactctaagaagggcaataggacccctttccagtacctgtctagctccgattctaagatcagctacgagaccttcaagaagcacatcctgaatctggccaagggcaagggccgcatctctaagaccaagaaggagtacctgctggaggagcgggacatcaacagattcagcgtgcagaaggacttcatcaaccggaatctggtggacaccagatacgccacacgcggcctgatgaatctgctgcggtcctatttcagagtgaacaatctggatgtgaaggtgaagagcatcaacggcggcttcacctcctttctgcggagaaagtggaagtttaagaaggagagaaacaagggctataagcaccacgccgaggatgccctgatcatcgccaatgccgacttcatctttaaggagtggaagaagctggacaaggccaagaaagtgatggagaaccagatgttcgaggagaagcaggccgagagcatgcccgagatcgagaccgagcaggagtacaaggagattttcatcacacctcaccagatcaagcacatcaaggacttcaaggactacaagtattcccacagggtggataagaagcccaaccgcgagctgatcaatgacaccctgtattctacaaggaaggacgataagggcaataccctgatcgtgaacaatctgaacggcctgtacgacaaggataatgacaagctgaagaagctgatcaacaagagccccgagaagctgctgatgtaccaccacgatcctcagacatatcagaagctgaagctgatcatggagcagtacggcgacgagaagaacccactgtataagtactatgaggagaccggcaactacctgacaaagtattccaagaaggataatggccccgtgatcaagaagatcaagtactatggcaacaagctgaatgcccacctggacatcaccgacgattaccccaacagccggaataaggtggtgaagctgagcctgaagccatacaggttcgacgtgtacctggacaacggcgtgtataagtttgtgacagtgaagaatctggatgtgatcaagaaggagaactactatgaagtgaatagcaagtgctacgaggaggccaagaagctgaagaagatcagcaaccaggccgagttcatcgcctctttttacaacaatgacctgatcaagatcaatggcgagctgtatagagtgatcggcgtgaacaatgatctgctgaaccgcatcgaagtgaatatgatcgacatcacctaccgggagtatctggagaacatgaatgataagaggccccctcgcatcatcaagaccatcgcctctaagacacagagcatcaagaagtactctacagacatcctgggcaacctgtatgaggtgaagagcaagaagcaccctcagatcatcaagaagggc

SEQ ID NO: 915-1000 - skipped (86 entries)

SEQ ID NO: 1001 - DNA, 19 bp, synthetic construct
  misc_feature 1..19: note = Synthetic
  source 1..19: mol_type = other DNA
tgtccctcctggatcgcag

SEQ ID NO: 1002 - DNA, 18 bp, synthetic construct
  misc_feature 1..18: note = Synthetic
  source 1..18: mol_type = other DNA
gtccctcctggatcgcag

SEQ ID NO: 1003 - DNA, 17 bp, synthetic construct
  misc_feature 1..17: note = Synthetic
  source 1..17: mol_type = other DNA
tccctcctggatcgcag

SEQ ID NO: 1004-1100 - skipped (97 entries)

SEQ ID NO: 1101 - DNA, 19 bp, synthetic construct
  misc_feature 1..19: note = Synthetic
  source 1..19: mol_type = other DNA
gaatggtggctgacccaca

SEQ ID NO: 1102 - DNA, 18 bp, synthetic construct
  misc_feature 1..18: note = Synthetic
  source 1..18: mol_type = other DNA
aatggtggctgacccaca

SEQ ID NO: 1103 - DNA, 17 bp, synthetic construct
  misc_feature 1..17: note = Synthetic
  source 1..17: mol_type = other DNA
atggtggctgacccaca

SEQ ID NO: 1104-1200 - skipped (97 entries)

SEQ ID NO: 1201 - DNA, 19 bp, synthetic construct
  misc_feature 1..19: note = Synthetic
  source 1..19: mol_type = other DNA
accaaacaccagtcactga

SEQ ID NO: 1202 - DNA, 18 bp, synthetic construct
  misc_feature 1..18: note = Synthetic
  source 1..18: mol_type = other DNA
ccaaacaccagtcactga

SEQ ID NO: 1203 - DNA, 17 bp, synthetic construct
  misc_feature 1..17: note = Synthetic
  source 1..17: mol_type = other DNA
caaacaccagtcactga

SEQ ID NO: 1204-1299 - skipped (96 entries)

SEQ ID NO: 1300 - DNA, 22 bp, synthetic construct
  misc_feature 1..22: note = Synthetic
  source 1..22: mol_type = other DNA
atggcctctctgcaccccgcct

SEQ ID NO: 1301 - DNA, 22 bp, synthetic construct
  misc_feature 1..22: note = Synthetic
  source 1..22: mol_type = other DNA
gaccctgaccctgaccctgagg

SEQ ID NO: 1302 - DNA, 22 bp, synthetic construct
  misc_feature 1..22: note = Synthetic
  source 1..22: mol_type = other DNA
tctctgcaccccgcctcagggt

SEQ ID NO: 1303 - DNA, 22 bp, synthetic construct
  misc_feature 1..22: note = Synthetic
  source 1..22: mol_type = other DNA
caccccgcctcagggtcagggt

SEQ ID NO: 1304 - DNA, 22 bp, synthetic construct
  misc_feature 1..22: note = Synthetic
  source 1..22: mol_type = other DNA
ccatagagcccacttttggggg

SEQ ID NO: 1305 - DNA, 22 bp, synthetic construct
  misc_feature 1..22: note = Synthetic
  source 1..22: mol_type = other DNA
ctggttctcccacagggcccgc

SEQ ID NO: 1306 - DNA, 22 bp, synthetic construct
  misc_feature 1..22: note = Synthetic
  source 1..22: mol_type = other DNA
gaagaacccaaagttgtccctc

SEQ ID NO: 1307 - DNA, 22 bp, synthetic construct
  misc_feature 1..22: note = Synthetic
  source 1..22: mol_type = other DNA
ctgcgatccaggagggacaact

SEQ ID NO: 1308 - DNA, 22 bp, synthetic construct
  misc_feature 1..22: note = Synthetic
  source 1..22: mol_type = other DNA
ggagggacaactttgggttctt

SEQ ID NO: 1309-1399 - skipped (91 entries)

SEQ ID NO: 1400 - DNA, 22 bp, synthetic construct
  misc_feature 1..22: note = Synthetic
  source 1..22: mol_type = other DNA
gtgggtgcagaacctacaaaag

SEQ ID NO: 1401 - DNA, 22 bp, synthetic construct
  misc_feature 1..22: note = Synthetic
  source 1..22: mol_type = other DNA
gaagaacccaaagttgtccctc

SEQ ID NO: 1402 - DNA, 22 bp, synthetic construct
  misc_feature 1..22: note = Synthetic
  source 1..22: mol_type = other DNA
aagttgtccctcctggatcgca

SEQ ID NO: 1403 - DNA, 22 bp, synthetic construct
  misc_feature 1..22: note = Synthetic
  source 1..22: mol_type = other DNA
ttgtccctcctggatcgcagag

SEQ ID NO: 1404 - DNA, 22 bp, synthetic construct
  misc_feature 1..22: note = Synthetic
  source 1..22: mol_type = other DNA
tgtccctcctggatcgcagagg

SEQ ID NO: 1405 - DNA, 22 bp, synthetic construct
  misc_feature 1..22: note = Synthetic
  source 1..22: mol_type = other DNA
gtccctcctggatcgcagagga

SEQ ID NO: 1406 - DNA, 22 bp, synthetic construct
  misc_feature 1..22: note = Synthetic
  source 1..22: mol_type = other DNA
tccctcctggatcgcagaggag

SEQ ID NO: 1407 - DNA, 22 bp, synthetic construct
  misc_feature 1..22: note = Synthetic
  source 1..22: mol_type = other DNA
ctggatcgcagaggagggggca

SEQ ID NO: 1408 - DNA, 22 bp, synthetic construct
  misc_feature 1..22: note = Synthetic
  source 1..22: mol_type = other DNA
gatcgcagaggagggggcactg

SEQ ID NO: 1409 - DNA, 22 bp, synthetic construct
  misc_feature 1..22: note = Synthetic
  source 1..22: mol_type = other DNA
ctggtggcgcctgtctgcaaag

SEQ ID NO: 1410 - DNA, 22 bp, synthetic construct
  misc_feature 1..22: note = Synthetic
  source 1..22: mol_type = other DNA
gtctgcaaagctggttctccca

SEQ ID NO: 1411 - DNA, 22 bp, synthetic construct
  misc_feature 1..22: note = Synthetic
  source 1..22: mol_type = other DNA
tctgcaaagctggttctcccac

SEQ ID NO: 1412 - DNA, 22 bp, synthetic construct
  misc_feature 1..22: note = Synthetic
  source 1..22: mol_type = other DNA
tgagtcaccgccctcccagtgc

SEQ ID NO: 1413 - DNA, 22 bp, synthetic construct
  misc_feature 1..22: note = Synthetic
  source 1..22: mol_type = other DNA
gagtcaccgccctcccagtgcc

SEQ ID NO: 1414 - DNA, 22 bp, synthetic construct
  misc_feature 1..22: note = Synthetic
  source 1..22: mol_type = other DNA
cctcccagtgcctgggcacctg

SEQ ID NO: 1415 - DNA, 22 bp, synthetic construct
  misc_feature 1..22: note = Synthetic
  source 1..22: mol_type = other DNA
ctcccagtgcctgggcacctgt

SEQ ID NO: 1416 - DNA, 22 bp, synthetic construct
  misc_feature 1..22: note = Synthetic
  source 1..22: mol_type = other DNA
tcccagtgcctgggcacctgtt

SEQ ID NO: 1417 - DNA, 22 bp, synthetic construct
  misc_feature 1..22: note = Synthetic
  source 1..22: mol_type = other DNA
gcctgggcacctgttggggcag

SEQ ID NO: 1418 - DNA, 22 bp, synthetic construct
  misc_feature 1..22: note = Synthetic
  source 1..22: mol_type = other DNA
ggcacctgttggggcagctgga

SEQ ID NO: 1419 - DNA, 22 bp, synthetic construct
  misc_feature 1..22: note = Synthetic
  source 1..22: mol_type = other DNA
cctgttggggcagctggagagg

SEQ ID NO: 1420 - DNA, 22 bp, synthetic construct
  misc_feature 1..22: note = Synthetic
  source 1..22: mol_type = other DNA
gctggcgccgaacacctgcctg

SEQ ID NO: 1421 - DNA, 22 bp, synthetic construct
  misc_feature 1..22: note = Synthetic
  source 1..22: mol_type = other DNA
cacctgcctgtcggctgcgccc

SEQ ID NO: 1422 - DNA, 22 bp, synthetic construct
  misc_feature 1..22: note = Synthetic
  source 1..22: mol_type = other DNA
cgcccctggcagctgccccatg

SEQ ID NO: 1423 - DNA, 22 bp, synthetic construct
  misc_feature 1..22: note = Synthetic
  source 1..22: mol_type = other DNA
cagctgccccatgctggccctc

SEQ ID NO: 1424 - DNA, 22 bp, synthetic construct
  misc_feature 1..22: note = Synthetic
  source 1..22: mol_type = other DNA
tgctggccctcctggcttgccc

SEQ ID NO: 1425 - DNA, 22 bp, synthetic construct
  misc_feature 1..22: note = Synthetic
  source 1..22: mol_type = other DNA
cctcctggcttgccccaggcca

SEQ ID NO: 1426 - DNA, 22 bp, synthetic construct
  misc_feature 1..22: note = Synthetic
  source 1..22: mol_type = other DNA
cttgccccaggccacaggaaac

SEQ ID NO: 1427 - DNA, 22 bp, synthetic construct
  misc_feature 1..22: note = Synthetic
  source 1..22: mol_type = other DNA
ttgccccaggccacaggaaacc

SEQ ID NO: 1428 - DNA, 22 bp, synthetic construct
  misc_feature 1..22: note = Synthetic
  source 1..22: mol_type = other DNA
tgccccaggccacaggaaacca

SEQ ID NO: 1429 - DNA, 22 bp, synthetic construct
  misc_feature 1..22: note = Synthetic
  source 1..22: mol_type = other DNA
caggccacaggaaaccagggga

SEQ ID NO: 1430 - DNA, 22 bp, synthetic construct
  misc_feature 1..22: note = Synthetic
  source 1..22: mol_type = other DNA
aggccacaggaaaccaggggag

SEQ ID NO: 1431 - DNA, 22 bp, synthetic construct
  misc_feature 1..22: note = Synthetic
  source 1..22: mol_type = other DNA
gagagggccatagagcccactt

SEQ ID NO: 1432 - DNA, 22 bp, synthetic construct
  misc_feature 1..22: note = Synthetic
  source 1..22: mol_type = other DNA
agagggccatagagcccacttt

SEQ ID NO: 1433 - DNA, 22 bp, synthetic construct
  misc_feature 1..22: note = Synthetic
  source 1..22: mol_type = other DNA
gagggccatagagcccactttt

SEQ ID NO: 1434 - DNA, 22 bp, synthetic construct
  misc_feature 1..22: note = Synthetic
  source 1..22: mol_type = other DNA
agggccatagagcccacttttg

SEQ ID NO: 1435 - DNA, 22 bp, synthetic construct
  misc_feature 1..22: note = Synthetic
  source 1..22: mol_type = other DNA
gggccatagagcccacttttgg

SEQ ID NO: 1436 - DNA, 22 bp, synthetic construct
  misc_feature 1..22: note = Synthetic
  source 1..22: mol_type = other DNA
ccatagagcccacttttggggg

SEQ ID NO: 1437 - DNA, 22 bp, synthetic construct
  misc_feature 1..22: note = Synthetic
  source 1..22: mol_type = other DNA
catagagcccacttttgggggg

SEQ ID NO: 1438 - DNA, 22 bp, synthetic construct
  misc_feature 1..22: note = Synthetic
  source 1..22: mol_type = other DNA
cccacttttggggggagggtcc

SEQ ID NO: 1439 - DNA, 22 bp, synthetic construct
  misc_feature 1..22: note = Synthetic
  source 1..22: mol_type = other DNA
ttttggggggagggtcctagga

SEQ ID NO: 1440 - DNA, 22 bp, synthetic construct
  misc_feature 1..22: note = Synthetic
  source 1..22: mol_type = other DNA
tttggggggagggtcctaggag

SEQ ID NO: 1441 - DNA, 22 bp, synthetic construct
  misc_feature 1..22: note = Synthetic
  source 1..22: mol_type = other DNA
gcatgaccctgaccctgaccct

SEQ ID NO: 1442 - DNA, 22 bp, synthetic construct
  misc_feature 1..22: note = Synthetic
  source 1..22: mol_type = other DNA
tgaccctgaccctgaccctgag

SEQ ID NO: 1443 - DNA, 22 bp, synthetic construct
  misc_feature 1..22: note = Synthetic
  source 1..22: mol_type = other DNA
gaccctgaccctgaccctgagg

SEQ ID NO: 1444 - DNA, 22 bp, synthetic construct
  misc_feature 1..22: note = Synthetic
  source 1..22: mol_type = other DNA
accctgaccctgaccctgaggc

SEQ ID NO: 1445 - DNA, 22 bp, synthetic construct
  misc_feature 1..22: note = Synthetic
  source 1..22: mol_type = other DNA
tgaccctgaggcggggtgcaga

SEQ ID NO: 1446 - DNA, 22 bp, synthetic construct
  misc_feature 1..22: note = Synthetic
  source 1..22: mol_type = other DNA
tgaggcggggtgcagagaggcc

SEQ ID NO: 1447 - DNA, 22 bp, synthetic construct
  misc_feature 1..22: note = Synthetic
  source 1..22: mol_type = other DNA
gaggcggggtgcagagaggcca

SEQ ID NO: 1448 - DNA, 22 bp, synthetic construct
  misc_feature 1..22: note = Synthetic
  source 1..22: mol_type = other DNA
ctgggtgtgtctccttcttttg

SEQ ID NO: 1449 - DNA, 22 bp, synthetic construct
  misc_feature 1..22: note = Synthetic
  source 1..22: mol_type = other DNA
gagggacaactttgggttcttc

SEQ ID NO: 1450 - DNA, 22 bp, synthetic construct
  misc_feature 1..22: note = Synthetic
  source 1..22: mol_type = other DNA
ggagggacaactttgggttctt

SEQ ID NO: 1451 - DNA, 22 bp, synthetic construct
  misc_feature 1..22: note = Synthetic
  source 1..22: mol_type = other DNA
tgcgatccaggagggacaactt

SEQ ID NO: 1452 - DNA, 22 bp, synthetic construct
  misc_feature 1..22: note = Synthetic
  source 1..22: mol_type = other DNA
ctgcgatccaggagggacaact

SEQ ID NO: 1453 - DNA, 22 bp, synthetic construct
  misc_feature 1..22: note = Synthetic
  source 1..22: mol_type = other DNA
gccccctcctctgcgatccagg

SEQ ID NO: 1454 - DNA, 22 bp, synthetic construct
  misc_feature 1..22: note = Synthetic
  source 1..22: mol_type = other DNA
tgccccctcctctgcgatccag

SEQ ID NO: 1455 - DNA, 22 bp, synthetic construct
  misc_feature 1..22: note = Synthetic
  source 1..22: mol_type = other DNA
cagtgccccctcctctgcgatc

SEQ ID NO: 1456 - DNA, 22 bp, synthetic construct
  misc_feature 1..22: note = Synthetic
  source 1..22: mol_type = other DNA
gtgggagaaccagctttgcaga

SEQ ID NO: 1457 - DNA, 22 bp, synthetic construct
  misc_feature 1..22: note = Synthetic
  source 1..22: mol_type = other DNA
gcggtgactcacgcgggccctg

SEQ ID NO: 1458 - DNA, 22 bp, synthetic construct
  misc_feature 1..22: note = Synthetic
  source 1..22: mol_type = other DNA
ggcggtgactcacgcgggccct

SEQ ID NO: 1459 - DNA, 22 bp, synthetic construct
  misc_feature 1..22: note = Synthetic
  source 1..22: mol_type = other DNA
actgggagggcggtgactcacg

SEQ ID NO: 1460 - DNA, 22 bp, synthetic construct
  misc_feature 1..22: note = Synthetic
  source 1..22: mol_type = other DNA
cactgggagggcggtgactcac

SEQ ID NO: 1461 - DNA, 22 bp, synthetic construct
  misc_feature 1..22: note = Synthetic
  source 1..22: mol_type = other DNA
caggtgcccaggcactgggagg

SEQ ID NO: 1462 - DNA, 22 bp, synthetic construct
  misc_feature 1..22: note = Synthetic
  source 1..22: mol_type = other DNA
caacaggtgcccaggcactggg

SEQ ID NO: 1463 - DNA, 22 bp, synthetic construct
  misc_feature 1..22: note = Synthetic
  source 1..22: mol_type = other DNA
ccaacaggtgcccaggcactgg

SEQ ID NO: 1464 - DNA, 22 bp, synthetic construct
  misc_feature 1..22: note = Synthetic
  source 1..22: mol_type = other DNA
gccccaacaggtgcccaggcac

SEQ ID NO: 1465 - DNA, 22 bp, synthetic construct
  misc_feature 1..22: note = Synthetic
  source 1..22: mol_type = other DNA
tgccccaacaggtgcccaggca

SEQ ID NO: 1466 - DNA, 22 bp, synthetic construct
  misc_feature 1..22: note = Synthetic
  source 1..22: mol_type = other DNA
tccagctgccccaacaggtgcc

SEQ ID NO: 1467 - DNA, 22 bp, synthetic construct
  misc_feature 1..22: note = Synthetic
  source 1..22: mol_type = other DNA
ccagcctctccagctgccccaa

SEQ ID NO: 1468 - DNA, 22 bp, synthetic construct
  misc_feature 1..22: note = Synthetic
  source 1..22: mol_type = other DNA
ggcgcagccgacaggcaggtgt

SEQ ID NO: 1469 - DNA, 22 bp, synthetic construct
  misc_feature 1..22: note = Synthetic
  source 1..22: mol_type = other DNA
tgccaggggcgcagccgacagg

SEQ ID NO: 1470 - DNA, 22 bp, synthetic construct
  misc_feature 1..22: note = Synthetic
  source 1..22: mol_type = other DNA
cagctgccaggggcgcagccga

SEQ ID NO: 1471 - DNA, 22 bp, synthetic construct
  misc_feature 1..22: note = Synthetic
  source 1..22: mol_type = other DNA
ggccagcatggggcagctgcca

SEQ ID NO: 1472 - DNA, 22 bp, synthetic construct
  misc_feature 1..22: note = Synthetic
  source 1..22: mol_type = other DNA
gggccagcatggggcagctgcc

SEQ ID NO: 1473 - DNA, 22 bp, synthetic construct
  misc_feature 1..22: note = Synthetic
  source 1..22: mol_type = other DNA
agggccagcatggggcagctgc

SEQ ID NO: 1474 - DNA, 22 bp, synthetic construct
  misc_feature 1..22: note = Synthetic
  source 1..22: mol_type = other DNA
ggcaagccaggagggccagcat

SEQ ID NO: 1475 - DNA, 22 bp, synthetic construct
  misc_feature 1..22: note = Synthetic
  source 1..22: mol_type = other DNA
gggcaagccaggagggccagca

SEQ ID NO: 1476 - DNA, 22 bp, synthetic construct
  misc_feature 1..22: note = Synthetic
  source 1..22: mol_type = other DNA
ggggcaagccaggagggccagc

SEQ ID NO: 1477 - DNA, 22 bp, synthetic construct
  misc_feature 1..22: note = Synthetic
  source 1..22: mol_type = other DNA
ctgtggcctggggcaagccagg

SEQ ID NO: 1478 - DNA, 22 bp, synthetic construct
  misc_feature 1..22: note = Synthetic
  source 1..22: mol_type = other DNA
cctgtggcctggggcaagccag

SEQ ID NO: 1479 - DNA, 22 bp, synthetic construct
  misc_feature 1..22: note = Synthetic
  source 1..22: mol_type = other DNA
tttcctgtggcctggggcaagc

SEQ ID NO: 1480 - DNA, 22 bp, synthetic construct
  misc_feature 1..22: note = Synthetic
  source 1..22: mol_type = other DNA
ctcccctggtttcctgtggcct

SEQ ID NO: 1481 - DNA, 22 bp, synthetic construct
  misc_feature 1..22: note = Synthetic
  source 1..22: mol_type = other DNA
tctcccctggtttcctgtggcc

SEQ ID NO: 1482 - DNA, 22 bp, synthetic construct
  misc_feature 1..22: note = Synthetic
  source 1..22: mol_type = other DNA
ctctcccctggtttcctgtggc

SEQ ID NO: 1483 - DNA, 22 bp, synthetic construct
  misc_feature 1..22: note = Synthetic
  source 1..22: mol_type = other DNA
tggccctctcccctggtttcct

SEQ ID NO: 1484 - DNA, 22 bp, synthetic construct
  misc_feature 1..22: note = Synthetic
  source 1..22: mol_type = other DNA
gtgggctctatggccctctccc

SEQ ID NO: 1485 - DNA, 22 bp, synthetic construct
  misc_feature 1..22: note = Synthetic
  source 1..22: mol_type = other DNA
cctccccccaaaagtgggctct

SEQ ID NO: 1486 - DNA, 22 bp, synthetic construct
  misc_feature 1..22: note = Synthetic
  source 1..22: mol_type = other DNA
cctaggaccctccccccaaaag

SEQ ID NO: 1487 - DNA, 22 bp, synthetic construct
  misc_feature 1..22: note = Synthetic
  source 1..22: mol_type = other DNA
tcctaggaccctccccccaaaa

SEQ ID NO: 1488 - DNA, 22 bp, synthetic construct
  misc_feature 1..22: note = Synthetic
  source 1..22: mol_type = other DNA
tcatgctgggagctccctctcc

SEQ ID NO: 1489 - DNA, 22 bp, synthetic construct
  misc_feature 1..22: note = Synthetic
  source 1..22: mol_type = other DNA
agggtcagggtcagggtcatgc

SEQ ID NO: 1490 - DNA, 22 bp, synthetic construct
  misc_feature 1..22: note = Synthetic
  source 1..22: mol_type = other DNA
cagggtcagggtcagggtcatg

SEQ ID NO: 1491 - DNA, 22 bp, synthetic construct
  misc_feature 1..22: note = Synthetic
  source 1..22: mol_type = other DNA
accccgcctcagggtcagggtc

SEQ ID NO: 1492 - DNA, 22 bp, synthetic construct
  misc_feature 1..22: note = Synthetic
  source 1..22: mol_type = other DNA
caccccgcctcagggtcagggt

SEQ ID NO: 1493 - DNA, 22 bp, synthetic construct
  misc_feature 1..22: note = Synthetic
  source 1..22: mol_type = other DNA
ctctgcaccccgcctcagggtc

SEQ ID NO: 1494 - DNA, 22 bp, synthetic construct
  misc_feature 1..22: note = Synthetic
  source 1..22: mol_type = other DNA
tctctgcaccccgcctcagggt

SEQ ID NO: 1495 - DNA, 22 bp, synthetic construct
  misc_feature 1..22: note = Synthetic
  source 1..22: mol_type = other DNA
tggcctctctgcaccccgcctc

SEQ ID NO: 1496 - DNA, 22 bp, synthetic construct
  misc_feature 1..22: note = Synthetic
  source 1..22: mol_type = other DNA
atggcctctctgcaccccgcct

SEQ ID NO: 1497 - DNA, 22 bp, synthetic construct
  misc_feature 1..22: note = Synthetic
  source 1..22: mol_type = other DNA
ggggctcgaagggtccttgtag

SEQ ID NO: 1498 - DNA, 22 bp, synthetic construct
  misc_feature 1..22: note = Synthetic
  source 1..22: mol_type = other DNA
cgcgccagacgctccccagagc

SEQ ID NO: 1499 - skipped

SEQ ID NO: 1500 - DNA, 20 bp, synthetic construct
  misc_feature 1..20: note = Synthetic
  source 1..20: mol_type = other DNA
ggagacaggtctgggcacag

SEQ ID NO: 1501 - DNA, 20 bp, synthetic construct
  misc_feature 1..20: note = Synthetic
  source 1..20: mol_type = other DNA
gaggccaggagagtcattag

SEQ ID NO: 1502 - DNA, 20 bp, synthetic construct
  misc_feature 1..20: note = Synthetic
  source 1..20: mol_type = other DNA
agagaaggggagacagacag

SEQ ID NO: 1503 - DNA, 20 bp, synthetic construct
  misc_feature 1..20: note = Synthetic
  source 1..20: mol_type = other DNA
aagccaggagggccagcatg

SEQ ID NO: 1504 - DNA, 20 bp, synthetic construct
  misc_feature 1..20: note = Synthetic
  source 1..20: mol_type = other DNA
tcaccgccctcccagtgcct

SEQ ID NO: 1505 - DNA, 20 bp, synthetic construct
  misc_feature 1..20: note = Synthetic
  source 1..20: mol_type = other DNA
tgtgaggggttaaggctggg

SEQ ID NO: 1506 - protein, 9 aa, synthetic construct
  REGION 1..9: note = Synthetic: c-Myc NLS
PAAKKKKLD

SEQ ID NO: 1507 - DNA, 27 bp, synthetic construct
  misc_feature 1..27: note = Synthetic
  source 1..27: mol_type = other DNA
ccggcagctaagaaaaagaaactggat

SEQ ID NO: 1508 - protein, 4 aa, synthetic construct
  REGION 1..4: note = Synthetic
GSVD

SEQ ID NO: 1509 - protein, 4 aa, synthetic construct
  REGION 1..4: note = Synthetic
GSGS

SEQ ID NO: 1510 - protein, 4 aa, synthetic construct
  REGION 1..4: note = Synthetic
GSGS

(19) United States
(12) Patent Application Publication
(10) Pub. No.: US 2024/0344059 A1
(43) Pub. Date: Oct. 17, 2024

(54) Gene Editing of Regulatory Elements

(71) Applicant: Vertex Pharmaceuticals Incorporated, Boston, MA (US)

(72) Inventors: Guoxiang Ruan, Boston, MA (US); Jianming Liu, Boston, MA (US); Lingjun Rao, Boston, MA (US); Jinjin Zhu, Boston, MA (US)

(73) Assignee: Vertex Pharmaceuticals Incorporated, Boston, MA (US)

(21) Appl. No.: 18/435,805

(22) Filed: Feb. 7, 2024

Related U.S. Application Data
(63) Continuation of application No. PCT/US2022/039675, filed on Aug. 8, 2022.
(60) Provisional application No. 63/231,067, filed on Aug. 9, 2021.

(51) Int. Cl. C12N 15/11; C12N 9/22; C12N 15/90

(52) U.S. Cl. CPC C12N 15/11; C12N 9/22; C12N 15/907; C12N 2310/20

(57) ABSTRACT

Compositions and methods for disrupting and/or excising regulatory elements or portions thereof that are associated with genes that produce toxic proteins and transcripts are encompassed.

This application is a bypass continuation of PCT/US2022/039675, filed Aug. 8, 2022, which claims the benefit of priority to U.S. Provisional Application No. 63/231,067, filed Aug. 9, 2021, which is incorporated by reference in its entirety.

The instant application contains a Sequence Listing which has been submitted electronically in XML format and is hereby incorporated by reference in its entirety. Said XML copy, created on Jan. 17, 2024, is named 01245-0030-00US-SL, and is 543131 bytes in size.

### INTRODUCTION AND SUMMARY

Protein and RNA toxicity are a suspected cause of cell dysfunction and death in many diseases or disorders characterized by aberrant mRNA, DNA, and/or protein production. In some instances, protein-coding and non-coding RNAs are improperly regulated such that “toxic” transcripts are produced by the cell. Such events can lead to cell death and damage. In other instances, misregulation of RNA production can lead to abnormal protein production and interations, which can impede normal functioning of the cell. The production of these toxic RNA and proteins can also prevent and otherwise interfere with biogenesis of other essential mRNAs, which can disturb multiple downstream cellular processes, which ultimately causes complex and often multisystemic diseases, many of which are largely untreated. There is thus a need in the art for a therapeutic approach to prevent the production of toxic proteins and RNA in order to restore normal protein and RNA function.

Gene expression is normally a precisely controlled process through the integration of signals that act at gene regulatory elements such as promoters and enhancers. DNA regulatory elements recruit proteins known as transcription factors (TFs) in a sequence-dependent fashion, allowing cells to precisely control chromatin architecture, transcription initiation, and elongation. This disclosure provides compositions and methods for gene editing by disrupting regulatory elements that at least partially drive expression of genes associated with toxic RNA and protein (e.g., toxic proteins and transcripts or gain-of-function toxic proteins and transcripts) by using CRISPR gene editing.

In some of these neurological and neuromuscular diseases, repetitive DNA sequences, including trinucleotide repeats (TNRs) and other sequences with self-complementarity in or near various genes can lead to the production of the toxic RNA or proteins. Myotonic Dystrophy Type 1 (DM1), for example, is an autosomal dominant muscle disorder caused by an abnormal expansion of CTG trinucleotide repeat in the 3′ untranslated region (UTR) of human DM protein kinase gene (DMPK) gene, which leads to RNA foci and mis-splicing of genes important for muscle function. The disorder affects skeletal and smooth muscle as well as the eye, heart, endocrine system, and central nervous system, and causes muscle weakness, wasting, physical disablement, and shortened lifespan. As a consequence of the abnormal expansion of CTG tribucleotide repeat, mutant DMPK transcripts containing expanded CUG repeats are retained in nuclear foci and disrupt normal activities of RNA-binding proteins belonging to the muscleblind-like (MBNL) and CUGBP and ETR-3-like factors (CELF) families, which leads to mis-splicing of various genes important for muscle function.

Regulation of transcriptional initiation is a crucial step in the control of gene expression and commences when RNA polymerase binds at a transcription start site (TSS). In DM1, one possible way to reduce mutant DMPK transcript levels and thus ameliorate the DM1 disease phenotype could be to disrupt TSS in the DMPK gene. The muscle-specific transcription factor MyoD induces the expression of muscle genes by binding as a heterodimer to the conserved E-box motif in the transcriptional regulatory regions of target genes. The first intron of the DMPK gene was previously shown to function as a myogenic and cardiac enhancer and it contains four clustered E-box elements (Storbeck et al., J Biol Chem. 1998). Similar to TSS disruptions, ablation of these four E-boxes and/or other cis-regulatory elements in the DMPK promoter and intronic enhancer region could also reduce muscle-specific mutant DMPK expression.

For other diseases or disorders characterized by gain-of-function toxic proteins and transcripts, different genes and different regulatory elements are implicated in the abnormal production of RNA and/or proteins. Disruption or ablation of the regulatory elements driving expression of a gene should similarly reduce the abnormal expression, leading to treatment of the disease.

Accordingly, the following embodiments are provided:
Embodiment 1. A method of gene editing, comprising contacting a cell with
   a. one or more guide RNAs or nucleic acids encoding one or more guide RNAs; and
   b. an RNA-targeted endonuclease, or a nucleic acid encoding the RNA-targeted endonuclease,
      wherein the guide RNA comprises a guide sequence that directs the RNA-targeted endonuclease to or near one or more regulatory elements that at least partially drive expression of a gene, and wherein the method causes disruption and/or exision of at least a portion of the one or more regulatory elements, wherein the gene is selected from: DM1 protein kinase (DMPK), CCHC-type zinc finger nucleic acid binding protein (CNBP), transcription Factor 4 (TCF4), junctophilin 3 (JPH3), huntingtin (HTT), ataxin 1 (ATXN1), ataxin 2 (ATXN2), ataxin 3 (ATXN3), ataxin 7 (ATXN7), ATXN8 opposite strand lncRNA (ATXN8OS), androgen receptor (AR), atrophin 1 (ATN1), fragile X Mental Retardation 1 (FMR1), chromosome 9 open reading frame 72 (C90RF72), polyadenylate-binding protein 2 (PABP2), amyloid precursor protein (APP), presenilin 1 (PSEN1), presenilin 2 (PSEN2), apolipoprotein E4 (APOE4), and synuclein alpha (SNCA).
Embodiment 2. A method of treating a disease or disorder characterized by toxic proteins and/or transcripts in a human subject, comprising administering
   a. one or more guide RNAs or nucleic acids encoding one or more guide RNAs; and
   b. an RNA-targeted endonuclease, or a nucleic acid encoding the RNA-targeted endonuclease,
      wherein the guide RNA comprises a guide sequence that directs the RNA-targeted endonuclease to or near one or more regulatory elements that at least partially drive expression of a gene causing production of a toxic protein and/or producing toxic transcript, and wherein the method causes disruption and/or exision of at least a portion of the one or more regulatory elements, wherein the disruption and/or exision results in a reduction in the production of the toxic protein and/or transcript.
Embodiment 3. A method of treating DM1 in a human subject, comprising administering
   a. one or more guide RNAs or nucleic acids encoding one or more guide RNAs; and
   b. an RNA-targeted endonuclease, or a nucleic acid encoding the RNA-targeted endonuclease,
      wherein the guide RNA comprises a guide sequence that directs the RNA-targeted endonuclease to or near one or more regulatory elements that at least partially drive expression of the DMPK gene, and wherein the method causes disruption and/or exision of at least a portion of the one or more regulatory elements.
Embodiment 4. The method of embodiment 1 or 2, wherein the gene is associated with aberrant nucleotide repeats.
Embodiment 5. The method of embodiment 4, wherein the guide RNA does not excise the aberrant nucleotide repeats.
Embodiment 6. The method of embodiment 1 or 2, where the gene is DMPK.
Embodiment 7. The method of embodiment 6, wherein the one or more regulatory elements is a transcription start site (TSS).
Embodiment 8. The method of embodiment 6, wherein the one or more regulatory elements is an E-Box.
Embodiment 9. The method of embodiment 6, wherein the one or more regulatory elements is a TSS and an E-Box
Embodiment 10. The method of embodiment 1 or 2, wherein the gene is HTT.
Embodiment 11. The method of embodiment 10, wherein the one or more regulatory elements is selected from NF-κB binding domain, 20 bp direct repeats, transcription factor AP2, transcription factor Sp1, Huntington disease gene regulatory region-binding protein 1 and 2 (HDBP1 and HDBP2), uncharacterized regulatory region (UCRR).
Embodiment 12. The method of embodiment 1 or 2, wherein the gene is FMR1.
Embodiment 13. The method of embodiment 12, wherein the one or more regulatory elements is selected from 22-bp methylation sensitive element (MSE) and the single-nucleotide polymorphism (SNP) variant comprising a T/C at ss71651738.
Embodiment 14. The method of embodiment 1 or 2, wherein the gene is CCHC-type zinc finger nucleic acid binding protein (CNBP).
Embodiment 15. The method of embodiment 1 or 2, wherein the gene is transcription Factor 4 (TCF4).
Embodiment 16. The method of embodiment 1 or 2, wherein the gene is junctophilin 3 (JPH3).
Embodiment 17. The method of embodiment 1 or 2, wherein the gene is ataxin 1 (ATXN1).
Embodiment 18. The method of embodiment 1 or 2, wherein the gene is ataxin 2 (ATXN2).
Embodiment 19. The method of embodiment 1 or 2, wherein the gene is ataxin 3 (ATXN3).
Embodiment 20. The method of embodiment 1 or 2, wherein the gene is ataxin 7 (ATXN7).
Embodiment 21. The method of embodiment 1 or 2, wherein the gene is ATXN8 opposite strand lncRNA (ATXN8OS).
Embodiment 22. The method of embodiment 1 or 2, wherein the gene is androgen receptor (AR).
Embodiment 23. The method of embodiment 1 or 2, wherein the gene is atrophin 1 (ATN1).
Embodiment 24. The method of embodiment 1 or 2, wherein the gene is chromosome 9 open reading frame 72 (C90RF72).
Embodiment 25. The method of embodiment 1 or 2, wherein the gene is polyadenylate-binding protein 2 (PABP2).
Embodiment 26. The method of embodiment 1 or 2, wherein the gene is amyloid precursor protein (APP).
Embodiment 27. The method of embodiment 1 or 2, wherein the gene is presenilin 1 (PSEN1).
Embodiment 28. The method of embodiment 1 or 2, wherein the gene is presenilin 2 (PSEN2).
Embodiment 29. The method of embodiment 1 or 2, wherein the gene is apolipoprotein E4 (APOE4).
Embodiment 30. The method of embodiment 1 or 2, wherein the gene is and synuclein alpha (SNCA).
Embodiment 31. The method of embodiment 1 or 2, wherein the guide sequence directs the RNA-targeted endonuclease to a target sequence within within 100, 200, 300, 400, 500, 600, 700, 800, 900, 1000, 1100, 1200, 1300, 1400 or 1500 nucleotides of a regulatory element, or wherein a pair of guide RNAs comprising a pair of guide sequences directs the RNA-targeted endonuclease to two target sequences flanking a regulatory element, wherein at least one or both of the target sequences flanking the regulatory element are within 5, within 10, within 20, within 30, within 40, within 50 or within 100 nucleotides of the regulatory element.
Embodiment 32. The method of embodiment 1 or 2, comprising a pair of guide RNAs, wherein the pair of guide RNAs excises at least a portion of a regulatory element, wherein excised portion is less than 3000, 2500, 2200, 2000, 1800, 1500, 1400, 1300, 1200, 1100, 1000, 900, 800, 700, 600, 500, 400, 300, 200, 100, 50, or 25 nucleotides in length.
Embodiment 33. The method of embodiment 32, wherein the excised portion is less than 300 nucleotides in length.
Embodiment 34. The method of embodiment 32, wherein the excised portion is is less than 100 nucleotides in length.
Embodiment 35. The method of embodiment 21, wherein the excised portion is between 25-2000, 25-1500, 25-1000, 25-700, 25-400, 25-250, 25-100, 100-2000, 100-1500, 100-1000, 100-700, 100-400, 100-250, 250-2000, 250-1500, 250-1000, 250-700, 250-400, 400-2000, 400-1500, 400-1000, 400-700, 700-2000, 700-1500, 700-1000, 1000-2000, 1000-1500, or 1500-2000 nucleotides in length.
Embodiment 36. The method of any one of the preceding embodiments, wherein the regulatory element is not in the exon of the target gene.
Embodiment 37. The method of any one of the preceding embodiments, wherein the regulatory element is in an intron, and wherein the one or more guide RNAs directs the RNA-targeted endonuclease to a target sequence in the same intron.
Embodiment 38. The method of any one of the preceding embodiments, wherein the regulatory element is a transcription start site (TSS).
Embodiment 39. The method of any one of the preceding embodiments, wherein the regulatory element is an E-box.
Embodiment 40. The method of any one of the preceding embodiments comprising at least two guide RNAs, wherein the guide RNAs direct the RNA-targeted endonuclease to or near an E-box and/or a TSS.
Embodiment 41. The method of any one of the preceding embodiments, wherein the regulatory element is an enhancer.
Embodiment 42. The method of any one of the preceding embodiments, wherein the regulatory element is a promoter.
Embodiment 43. The method of any one of the preceding embodiments, wherein the RNA-targeted endonuclease is a Cas nuclease.
Embodiment 44. The method of embodiment 43, wherein the Cas nuclease is Cas9.
Embodiment 45. The method of embodiment 44, wherein the Cas9 nuclease is from Streptococcus pyogenes (spCas9).
Embodiment 46. The method of embodiment 44 wherein the Cas9 nuclease is from Staphylococcus aureus (saCas9).
Embodiment 47. The method of embodiment 44 wherein the Cas9 nuclease is from Staphylococcus lugdunensis (sluCas9).
Embodiment 48. The method of embodiment 43, wherein the Cas nuclease is a Cpf1 nuclease.
Embodiment 49. The method of any one of the preceding embodiments, further comprising administering a DNA-PK inhibitor.
Embodiment 50. The method of any one of the preceding embodiments, wherein no portion of the target gene that codes for the gene is excised.
Embodiment 51. The method of any one of the preceding embodiments, wherein the method does not completely eliminate expression of the target gene.
Embodiment 52. The method of any one of the preceding embodiments, wherein the method reduces expression of the target gene by at least 10%, at least 20%, at least 30%, at least 40%, or at least 50%.
Embodiment 53. The method of any of the preceding embodiments, wherein the guide RNA is an sgRNA.
Embodiment 54. The method of embodiment 53, wherein the sgRNA is modified.
Embodiment 55. The method of embodiment 54, wherein the modification alters one or more 2′ positions and/or phosphodiester linkages.
Embodiment 56. The method of any one of embodiments 53-55, wherein the modification alters one or more, or all, of the first three nucleotides of the sgRNA.
Embodiment 57. The method of any one of embodiments 53-56, wherein the modification alters one or more, or all, of the last three nucleotides of the sgRNA.
Embodiment 58. The method of any one of embodiments 53-57, wherein the modification includes one or more of a phosphorothioate modification, a 2′-OMe modification, a 2′-O-MOE modification, a 2′-F modification, a 2′-O-methine-4′ bridge modification, a 3′-thiophosphonoacetate modification, or a 2′-deoxy modification.
Embodiment 59. The method of any one of the preceding embodiments, wherein the guide RNA is associated with a lipid nanoparticle (LNP), lipoplex, or a viral vector.
Embodiment 60. The method of embodiment 59, wherein the viral vector is an adeno-associated virus vector, a lentiviral vector, an integrase-deficient lentiviral vector, an adenoviral vector, a vaccinia viral vector, an alphaviral vector, or a herpes simplex viral vector.
Embodiment 61. The method of embodiment 60, wherein the viral vector is an adeno-associated virus (AAV) vector.
Embodiment 62. The method of embodiment 61, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
Embodiment 63. The method of embodiment 62, wherein the AAV vector is an AAV serotype 9 vector.
Embodiment 64. The method of embodiment 62, wherein the AAV vector is an AAVrh10 vector.
Embodiment 65. The method of embodiment 62, wherein the AAV vector is an AAVrh74 vector.
Embodiment 66. The method of any one of embodiments 53-65, comprising a viral vector, wherein the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.
Embodiment 67. The method of any one of the preceding embodiments, comprising at least two guide RNAs, wherein two guide RNAs act as a pair to excise the regulatory element that at least partially drives expression of a gene.
Embodiment 68. The method of any one of the preceding embodiments, comprising at least two guide RNAs, wherein the two guide RNAs act as a pair to excise one or more exons from the gene.
Embodiment 69. The method of embodiment 68, wherein the method excises one or more exons from the gene and results in a different splice form of the gene than if the one or more exons had not been excised.
Embodiment 70. The method of embodiment 69, wherein the different splice form of the gene is expressed at a lower level than the gene if the one or more exons had not been excised.
Embodiment 71. The method of any one of embodiments 68-70, wherein the method excises all or most of exon 1 of the DMPK gene.
Embodiment 72. The method of embodiment 71, wherein the method excises all or most of intron 1 of the DMPK gene.
Embodiment 73. The method of embodiment 3, wherein the regulatory element is TSS and a pair of guide RNAs is administered, wherein the pair of guide RNAs comprise a first guide RNA comprising at least 17, 18, 19, or 20 contiguous nucleotides of a first guide sequence selected from any one of SEQ ID NOs: 1, 2, or 3, and a second guide RNA comprising at least 17, 18, 19, or 20 contiguous nucleotides of a second guide sequence selected from any one of SEQ ID NOs: 4, 5, or 6.
Embodiment 74. The method of embodiment 3, wherein the regulatory element is TSS and a pair of guide RNAs is administered, wherein the pair of guide RNAs comprise a first guide RNA comprising a first guide sequence that binds to a nucleotide sequence that is complementary to any one of SEQ ID NOs: 1, 2, or 3, and a second guide RNA comprising a second guide sequences that binds to a nucleotide sequence that is complementary to any one of SEQ ID NOs: 4, 5, or 6.
Embodiment 75. The method of embodiment 3, wherein the regulatory element is an E-box and a pair of guide RNAs is administered, wherein the pair of guide RNAs comprise a first guide RNA comprising at least 17, 18, 19, or 20 contiguous nucleotides of a first guide sequence selected from any one of SEQ ID NOs: 7, 8, or 9, and a second guide RNA comprising at least 17, 18, 19, or 20 contiguous nucleotides of a second guide sequence selected from any one of SEQ ID NOs: 10, 11, or 12.
Embodiment 76. The method of embodiment 3, wherein the regulatory element is and E-box and a pair of guide RNAs is administered, wherein the pair of guide RNAs comprise a first guide RNA comprising a first guide sequence that binds to a nucleotide sequence that is complementary to any one of SEQ ID NOs: 7, 8, or 9, and a second guide RNA comprising a second guide sequences that binds to a nucleotide sequence that is complementary to any one of SEQ ID NOs: 10, 11, or 12.
Embodiment 77. The method of embodiment 3, wherein a pair of guide RNAs is administered, wherein the pair of guide RNAs comprise a first guide RNA comprising a first guide sequence that binds to a nucleotide sequence that is complementary to any one of SEQ ID NOs: 1300, 1301, 1302, 1303, and 1304, and a second guide RNA comprising a second guide sequences that binds to a nucleotide sequence that is complementary to any one of SEQ ID NOs: 1305, 1306, 1307, and 1308.
Embodiment 78. The method of embodiment 3, wherein a pair of guide RNAs is administered, wherein the pair of guide RNAs comprise a first guide RNA comprising a first guide sequence that binds to a nucleotide sequence that is complementary to any one of SEQ ID NOs: 1420, 1428, and 1475, and a second guide RNA comprising a second guide sequences that binds to a nucleotide sequence that is complementary to any one of SEQ ID NOs: 1402, 1411, and 1463.
Embodiment 79. The method of embodiment 3, and wherein
   a. a first pair of guide RNAs is administered, wherein the first pair of guide RNAs comprise at least 17, 18, 19, or 20 contiguous nucleotides of a first guide sequence selected from any one of SEQ ID NOs: 1, 2, or 3, and at least 17, 18, 19, or 20 contiguous nucleotides of a second guide sequence selected from any one of SEQ ID NOs: 4, 5, or 6; and
   b. a second pair of guide RNAs is administered, wherein the pair of guide RNAs comprise at least 17, 18, 19, or 20 contiguous nucleotides of a first guide sequence selected from any one of SEQ ID NOs: 7, 8, or 9, and at least 17, 18, 19, or 20 contiguous nucleotides of a second guide sequence selected from any one of SEQ ID NOs: 10, 11, or 12.
Embodiment 80. The method of embodiment 3, and wherein
   a. a first pair of guide RNAs is administered, wherein the first pair of guide RNAs comprise at least 17, 18, 19, 20, 21, or 22 contiguous nucleotides of a first guide sequence selected from any one of SEQ ID NOs: 1300, 1301, 1302, 1303, and 1304, and at least 17, 18, 19, 20, 21, or 22 contiguous nucleotides of a second guide sequence selected from any one of SEQ ID NOs: 1305, 1306, 1307, and 1308; and
   b. a second pair of guide RNAs is administered, wherein the pair of guide RNAs comprise at least 17, 18, 19, 20, 21, or 22 contiguous nucleotides of a first guide sequence selected from any one of SEQ ID NOs: 1300, 1301, 1302, 1303, and 1304, and at least 17, 18, 19, 20, 21, or 22 contiguous nucleotides of a second guide sequence selected from any one of SEQ ID NOs: 1305, 1306, 1307, and 1308.
Embodiment 81. The method of embodiment 3, and wherein
   a. a first pair of guide RNAs is administered, wherein the first pair of guide RNAs comprise at least 17, 18, 19, 20, 21, or 22 contiguous nucleotides of a first guide sequence selected from any one of SEQ ID NOs: 1420, 1428, and 1475, and at least 17, 18, 19, 20, 21, or 22 contiguous nucleotides of a second guide sequence selected from any one of SEQ ID NOs: 1402, 1411, and 1463; and
   b. a second pair of guide RNAs is administered, wherein the pair of guide RNAs comprise at least 17, 18, 19, 20, 21, or 22 contiguous nucleotides of a first guide sequence selected from any one of SEQ ID NOs: 1420, 1428, and 1475, and at least 17, 18, 19, 20, 21, or 22 contiguous nucleotides of a second guide sequence selected from any one of SEQ ID NOs: 1402, 1411, and 1463.
Embodiment 82. The method of embodiment 2, wherein
   a. if the gene is DMPK and a subject has myotonic dystrophy type 1 (DM1), then administration to the subject leads to treatment of DM1;
   b. if the gene is CNBP and a subject has myotonic dystrophy type 2 (DM2), then administration to the subject leads to treatment of DM2;
   c. if the gene is TCF4 and a subject has Fuchs endothelial corneal dystrophy (FECD), then administration to the subject leads to treatment of FECD;
   d. if the gene is JPH3 and a subject has Huntington disease-like 2 (HDL2), then administration to the subject leads to treatment of HDL2;
   e. if the gene is HTT and a subject has Huntington disease (HD), then administration to the subject leads to treatment of HD;
   f. if the gene is ATXN and a subject has Spinocerebellar ataxia (SCA), then administration to the subject leads to treatment of SCA;
   g. if the gene is AR and a subject has Spinal and bulbar muscular atrophy (SBMA), then administration to the subject leads to treatment of SBMA;
   h. if the gene is ATN1 and a subject has Dentatorubral-pallidoluysian atrophy (DRPLA), then administration to the subject leads to treatment of DRPLA;
   i. if the gene is FMR1 and a subject has Fragile X-associated tremor/ataxia syndrome (FXTAS), then administration to the subject leads to treatment of FXTAS;
   j. if the gene is C900RF72 and a subject has Frontotemporal dementia (FTD) or amyotrophic lateral sclerosis (ALS), then administration to the subject leads to treatment of FTD or ALS;
   k. if the gene is PABP2 and a subject has oculopharyngeal muscular dystrophy (OMPD), then administration to the subject leads to the treatment of OPMD;
   l. if the gene is APP, PSEN1, PSEN2, or APOE4, and a subject has Alzheimer's Disease (AD), then administration to the subject leads to treatment of AD; and
   m. if the gene is SNCA and a subject has Parkinson's Disease (PD), then administration to the subject leads to treatment of PD.
Embodiment 83. A composition comprising a first guide RNA and a second guide RNA, wherein the first guide RNA directs an RNA-targeted endonuclease to or near the 3′ end of a regulatory element that at least partially drives expression of a gene that produces a toxic protein and/or toxic transcript, and wherein the second guide RNA directs a RNA-targeted endonuclease to or near the 5′ end the regulatory element.
Embodiment 84. The composition of embodiment 83, wherein the regulatory element is in an intron.
Embodiment 85. The composition of embodiment 83, wherein the regulatory element is a transcription start (TSS) site.
Embodiment 86. The composition of embodiment 83, wherein the regulatory element is an intronic E-box site.
Embodiment 87. The composition of embodiment 83, wherein the regulatory element is in exon 1 of the DMPK gene.
Embodiment 88. The composition of embodiment 83, comprising more than one regulatory element, wherein the regulatory elements are in intron 1 and exon 1 of the DMPK gene. Embodiment 89. The composition of embodiment 83, wherein the gene is HTT and the regulatory element a cis-regulatory element for regulating gene expression.
Embodiment 90. The composition of embodiment 89, wherein the regulatory element is an NF-κB binding domain, 20 bp direct repeats, AP2, Sp1, HDBP1/2, or UCRR.
Embodiment 91. The composition of embodiment 83, wherein the gene is FMR1 and the regulatory element is a 22-bp methylation sensitive element (MSE).
Embodiment 92. The composition of embodiment 83, wherein first and second guide RNAs function to excise at least a portion of a regulatory element, wherein excised portion is less than 3000, 2500, 2200, 2000, 1800, 1500, 1400, 1300, 1200, 1100, 1000, 900, 800, 700, 600, 500, 400, 300, 200, 100, 50, or 25 nucleotides in length.
Embodiment 93. The composition of embodiment 83, wherein the excised portion is less than 300 nucleotides in length.
Embodiment 94. The composition of embodiment 83, wherein the excised portion is is less than 100 nucleotides in length.
Embodiment 95. The composition of embodiment 83, wherein the excised portion is between 25-2000, 25-1500, 25-1000, 25-700, 25-400, 25-250, 25-100, 100-2000, 100-1500, 100-1000, 100-700, 100-400, 100-250, 250-2000, 250-1500, 250-1000, 250-700, 250-400, 400-2000, 400-1500, 400-1000, 400-700, 700-2000, 700-1500, 700-1000, 1000-2000, 1000-1500, or 1500-2000 nucleotides in length.
Embodiment 96. The composition of any one of embodiments 83-95, wherein the guide RNA is associated with a lipid nanoparticle (LNP) or lipoplex or one or more viral vectors.
Embodiment 97. The composition of embodiment 96 comprising one viral vector.
Embodiment 98. The composition of embodiment 96 comprising more than one viral vector, wherein a first vector comprises one or more guide RNAs and a second vector comprises a nucleic acid encoding an RNA-targeted endonuclease.
Embodiment 99. The composition of embodiment 96 comprising one viral vector comprising at least one guide RNA and a nucleic acid encoding an RNA-targeted endonuclease.
Embodiment 100. The composition of embodiment 98 or 99, wherein the RNA-targeted endonuclease is a Cas nuclease.
Embodiment 101. The composition of embodiment 100, wherein the Cas nuclease is Cas9. Embodiment 102. The composition of embodiment 101, wherein the Cas9 nuclease is from Streptococcus pyogenes (spCas9).
Embodiment 103. The composition of embodiment 101, wherein the Cas9 nuclease is from Staphylococcus aureus (saCas9).
Embodiment 104. The composition of embodiment 101, wherein the Cas9 nuclease is from Staphylococcus lugdunensis (sluCas9).
Embodiment 105. The composition of embodiment 100, wherein the Cas nuclease is a Cpf1 nuclease.
Embodiment 106. The composition of any one of embodiments 96-105, wherein the viral vector is an adeno-associated virus (AAV) vector.
Embodiment 107. The composition of embodiment 106, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
Embodiment 108. The composition of any one of embodiments 83-107, wherein
   a. the first guide RNA comprises at least one guide sequence selected from:
      i. at least 17, 18, 19, or 20 contiguous nucleotides of any one of SEQ ID NOs: 1, 2, or 3;
      ii. at least 17, 18, 19, or 20 contiguous nucleotides of a sequence complementary to any one of SEQ ID NOs: 1, 2, or 3; or
      iii. a sequence that binds to a sequence that is complementary to any one of SEQ ID NOs: 1, 2 or 3 and
   b. the second guide RNA comprises at least one guide sequence selected from:
      i. at least 17, 18, 19, or 20 contiguous nucleotides of any one of SEQ ID NOs: 4, 5, or 6;
      ii. at least 17, 18, 19, or 20 contiguous nucleotides of a sequence complementary to any one of SEQ ID NOs: 4, 5, or 6, or
      iii. a sequence that binds to a sequence that is complementary to any one of SEQ ID Nos: 4, 5 or 6.
Embodiment 109. The composition of any one of embodiments 83-107, wherein
   a. the first guide RNA comprises at least one guide sequence selected from:
      i. at least 17, 18, 19, or 20 contiguous nucleotides of any one of SEQ ID NOs: 7, 8, or 9;
      ii. at least 17, 18, 19, or 20 contiguous nucleotides of a sequence complementary to any one of SEQ ID NOs: 7, 8, or 9; or
      iii. a sequence that binds to a sequence that is complementary to any one of SEQ ID NOs: 7, 8 or 9 and
   b. the second guide RNA comprises at least one guide sequence selected from:
      i. at least 17, 18, 19, or 20 contiguous nucleotides of any one of SEQ ID NOs: 10, 11, or 12;
      ii. at least 17, 18, 19, or 20 contiguous nucleotides of a sequence complementary to any one of SEQ ID NOs: 10, 11, or 12; or
      iii. a sequence that binds to a sequence that is complementary to any one of SEQ ID NOs: 10, 11 or 12.
Embodiment 110. A composition comprising:
   a. at least one guide RNA comprising at least one guide sequence selected from:
      i. at least 17, 18, 19, or 20 contiguous nucleotides of any one of SEQ ID NOs: 1, 2, or 3;
      ii. at least 17, 18, 19, or 20 contiguous nucleotides of a sequence complementary to any one of SEQ ID NOs: 1, 2, or 3;
      iii. at least 17, 18, 19, or 20 contiguous nucleotides of any one of SEQ ID NOs: 4, 5, or 6;
      iv. at least 17, 18, 19, or 20 contiguous nucleotides of a sequence complementary to any one of SEQ ID NOs: 4, 5, or 6;
      v. at least 17, 18, 19, or 20 contiguous nucleotides of any one of SEQ ID NOs: 7, 8, or 9;
      vi. at least 17, 18, 19, or 20 contiguous nucleotides of a sequence complementary to any one of SEQ ID NOs: 7, 8, or 9;
      vii. at least 17, 18, 19, or 20 contiguous nucleotides of any one of SEQ ID NOs: 10, 11, or 12;
      viii. at least 17, 18, 19, or 20 contiguous nucleotides of a sequence complementary to any one of SEQ ID NOs: 10, 11, or 12; or
      ix. at least 17, 18, 19, 20, 21, or 22 contiguous nucleotides of any one of SEQ ID NOs: 1300, 1301, 1302, 1303, or 1304;
      x. at least 17, 18, 19, 20, 21, or 22 contiguous nucleotides of a sequence complementary to any one of SEQ ID NOs: 1300, 1301, 1302, 1303, or 1304;
      xi. at least 17, 18, 19, 20, 21, or 22 contiguous nucleotides of any one of SEQ ID NOs: 1305, 1306, 1307, or 1308;
      xii. at least 17, 18, 19, 20, 21, or 22 contiguous nucleotides of a sequence complementary to any one of SEQ ID NOs: 1305, 1306, 1307, or 1308;
      xiii. at least 17, 18, 19, 20, 21, or 22 contiguous nucleotides of any one of SEQ ID NOs: 1420, 1428, or 1475;
      xiv. at least 17, 18, 19, 20, 21, or 22 contiguous nucleotides of a sequence complementary to any one of SEQ ID NOs: 1420, 1428, or 1475;
      xv. at least 17, 18, 19, 20, 21, or 22 contiguous nucleotides of any one of SEQ ID NOs: 1402, 1411, or 1463;
      xvi. at least 17, 18, 19, 20, 21, or 22 contiguous nucleotides of a sequence complementary to any one of SEQ ID NOs: 1402, 1411, or 1463; or
   b. a pair of guide RNAs comprising:
      i. a first guide RNA comprising least 17, 18, 19, or 20 contiguous nucleotides of any one of SEQ ID NOs: 1, 2, or 3, or a sequence comprising least 17, 18, 19, or 20 contiguous nucleotides of a sequence that is complementary to any one of SEQ ID NOs: 1, 2, or 3; and a second guide RNA comprising at least 17, 18, 19, or 20 contiguous nucleotides of any one of SEQ ID NOs: 4, 5, or 6, or a sequence comprising least 17, 18, 19, or 20 contiguous nucleotides of a sequence that is complementary to any one of SEQ ID NOs: 4, 5, or 6;
      ii. a first guide RNA comprising at least 17, 18, 19, or 20 contiguous nucleotides of any one of SEQ ID NOs: 7, 8, or 9, or a sequence comprising least 17, 18, 19, or 20 contiguous nucleotides of a sequence that is complementary to any one of SEQ ID NOs: 7, 8, or 9; and a second guide RNA comprising at least 17, 18, 19, or 20 contiguous nucleotides of of any one of SEQ ID NOs: 10, 11, or 12, or a sequence comprising least 17, 18, 19, or 20 contiguous nucleotides of a sequence that is complementary to any one of SEQ ID NOs: 10, 11, or 12;
      iii. a first guide RNA comprising at least 17, 18, 19, 20, 21, or 22 contiguous nucleotides of any one of SEQ ID NOs: 1300, 1301, 1302, 1303, or 1304, or a sequence comprising least 17, 18, 19, 20, 21, or 22 contiguous nucleotides of a sequence that is complementary to any one of SEQ ID NOs: 1300, 1301, 1302, 1303, or 1304; and a second guide RNA comprising at least 17, 18, 19, 20, 21, or 22 contiguous nucleotides of any one of SEQ ID NOs: 1305, 1306, 1307, or 1308, or a sequence comprising least 17, 18, 19, 20, 21, or 22 contiguous nucleotides of a sequence that is complementary to any one of SEQ ID NOs: 1305, 1306, 1307, or 1308;
      iv. a first guide RNA comprising at least 17, 18, 19, 20, 21, or 22 contiguous nucleotides of any one of SEQ ID NOs: 1420, 1428, or 1475, or a sequence comprising least 17, 18, 19, 20, 21, or 22 contiguous nucleotides of a sequence that is complementary to any one of SEQ ID NOs: 1420, 1428, or 1475; and a second guide RNA comprising at least 17, 18, 19, 20, 21, or 22 contiguous nucleotides of of any one of SEQ ID NOs: 1420, 1428, or 1475, or a sequence comprising least 17, 18, 19, 20, 21, or 22 contiguous nucleotides of a sequence that is complementary to any one of SEQ ID NOs: 1420, 1428, or 1475; or
   c. two pairs of guide RNAs comprising 1) a first pair of guide RNAs selected from any one of the guide RNA pairs of (b)(i); and 2) a second pair of guide RNAs selected from any one of the guide RNA pairs of (b)(ii); or
   d. at least one guide RNA comprising at least one guide sequence that binds to a target sequence that is at least partially complementary to any one of SEQ ID NOs: 1-12, 1300-1308, or 1400-1496.
Embodiment 111. The composition of any one of embodiments 83-107, wherein
   a. the first guide RNA comprises at least one guide sequence selected from:
      i. at least 17, 18, 19, 20, 21, or 22 contiguous nucleotides of any one of SEQ ID NOs: 1300, 1301, 1302, 1303, or 1304;
      ii. at least 17, 18, 19, 20, 21, or 22 contiguous nucleotides of a sequence complementary to any one of SEQ ID NOs: 1300, 1301, 1302, 1303, or 1304; or
      iii. a sequence that binds to a sequence that is complementary to any one of SEQ ID NOs: 1300, 1301, 1302, 1303, or 1304 and
   b. the second guide RNA comprises at least one guide sequence selected from:
      i. at least 17, 18, 19, 20, 21, or 22 contiguous nucleotides of any one of SEQ ID NOs: 1305, 1306, 1307, or 1308;
      ii. at least 17, 18, 19, 20, 21, or 22 contiguous nucleotides of a sequence complementary to any one of SEQ ID NOs: 1305, 1306, 1307, or 1308, or
      iii. a sequence that binds to a sequence that is complementary to any one of SEQ ID Nos: 1305, 1306, 1307, or 1308.
Embodiment 112. The composition of any one of embodiments 83-107, wherein
   a. the first guide RNA comprises at least one guide sequence selected from:
      i. at least 17, 18, 19, 20, 21, or 22 contiguous nucleotides of any one of SEQ ID NOs: 1420, 1428, or 1475;
      ii. at least 17, 18, 19, 20, 21, or 22 contiguous nucleotides of a sequence complementary to any one of SEQ ID NOs: 1420, 1428, or 1475; or
      iii. a sequence that binds to a sequence that is complementary to any one of SEQ ID NOs: 1420, 1428, or 1475 and
   b. the second guide RNA comprises at least one guide sequence selected from:
      i. at least 17, 18, 19, 20, 21, or 22 contiguous nucleotides of any one of SEQ ID NOs: 1402, 1411, or 1463;
      ii. at least 17, 18, 19, 20, 21, or 22 contiguous nucleotides of a sequence complementary to any one of SEQ ID NOs: 1402, 1411, or 1463; or
      iii. a sequence that binds to a sequence that is complementary to any one of SEQ ID NOs: 1402, 1411, or 1463.
Embodiment 113. The composition of any one of embodiments 108-112, wherein the guide RNA is associated with a lipid nanoparticle (LNP) or one or more viral vectors.
Embodiment 114. The composition of embodiment 113 comprising one viral vector.
Embodiment 115. The composition of embodiment 113 comprising more than one viral vector, wherein a first vector comprises one or more guide RNAs and a second vector comprises a nucleic acid encoding spCas9.
Embodiment 116. The method of any one of embodiments 1-83, comprising administering a DNA-PKi.
Embodiment 117. A composition comprising any one or more of the guides disclosed in Tables, 3A, 3B, 5A, 5B, 5C, 6A or 6B.
Embodiment 118. A composition comprising a guide sequence selected from any one of SEQ ID Nos: 1-11.
Embodiment 119. A composition comprising a guide sequence selected from any one of SEQ ID Nos: 1-11 and a SpCas9 endonuclease or a nucleic acid encoding the endonuclease.
Embodiment 120. A composition comprising a guide sequence selected from any one of SEQ ID Nos: 1300-1308.
Embodiment 121. A composition comprising a guide sequence selected from any one of SEQ ID Nos: 1300-1308 and SaCas9 endonuclease or a nucleic acid encoding the endonuclease.
Embodiment 122. A composition comprising a guide sequence selected from any one of SEQ ID Nos: 1400-1496.
Embodiment 123. A composition comprising a guide sequence selected from any one of SEQ ID Nos: 1400-1496 and a SluCas9 endonuclease or a nucleic acid encoding the endonuclease.
Embodiment 124. A composition comprising a guide sequence selected from any one of SEQ ID Nos: 1420, 1428, 1475, 1402, 1411, and 1463.
Embodiment 125. A composition comprising a guide sequence selected from any one of SEQ ID Nos: 1420, 1428, 1475, 1402, 1411, and 1463 and a SluCas9 endonuclease or a nucleic acid encoding the endonuclease.
Embodiment 126. Use of any one of the compositions of claims 117-126 to treat a disease or disorder.

### BRIEF DESCRIPTION OF THE DRAWINGS

FIG. 1 shows a schematic of an example of the DMPK TSS and/or transcriptional regulatory elements-targeted gene editing therapeutic approach for DM1. CRISPR gene editing may be used to ablate the TSS and/or important transcriptional regulatory elements of the human DMPK gene.

FIG. 2 shows the location of 12 selected SpCas9 sgRNAs to delete the TSS (6 sgRNAs), and to delete the four E-boxes (6 sgRNAs). The TU (TU1, TU2, or TU3) sgRNAs and ED (ED1, ED2, or ED3) sgRNAs were also paired to delete both TSS and E-boxes.

FIG. 3 shows the experimental design of the experiments described in the Examples.

FIGS. 4A-4C show the results of verification of paired sgRNAs-induced excision by PCR amplification from experiments described in the Examples. Excision was verified by PCR amplification of genomic DNA prepared from DM1 patient myoblasts 72 h post nucleofection. All 27 sgRNA pairs targeting TSS (FIG. 4A), E-box (FIG. 4B), or TSS+E-box (FIG. 4C) showed downshifted bands compare to the band from the mock control (last lane in each gel picture).

FIGS. 5A-5B show reduction of DMPK transcript level by TSS and/or E-boxes targeted gene editing in DM1 patient myoblasts (FIG. 5A) and myotubes (FIG. 5B). DMPK transcript level was quantified by ddPCR and presented as mean±SD (n=3-6). All the samples were treated with 3 uM of DNA-PKi for 24 h.

FIGS. 6A-6B show the frequency of CUG-foci-free myoblast nuclei induced by TSS and/or E-boxes targeted gene editing in primary DM1 patient myoblasts (FIG. 6A) or myotubes (FIG. 6B). The data are presented as mean±SD (n=3-6). DM1 mock served as a negative control, and primary healthy myoblasts served as a positive control. All the myoblast samples were treated with DNA-PKi for 24 h. The myotube samples were treated with DNA-PKi for 24 h (solid bars) or without DNA-PKi (striated bars).

FIGS. 7A-7C show correction of mis-splicing by TSS and/or E-boxes targeted gene editing in primary DM1 patient myotubes. Percent of spliced-in (PSI) of BIN1 (FIG. 7A), KIF13a (FIG. 7B) and DMD (FIG. 7C) was assessed by ddPCR and presented as mean±SD (n=3-6). The PSI of all 3 genes was increased compare to DM1 mock control upon ablation of TSS and/or E-box region. All the samples were treated with 3 uM of DNA-PKi for 24 h.

FIG. 8 shows the selection of saCas9 and sluCas9 sgRNAs for the E-box excision in vitro study in primary DM1 patient myoblasts. All sgRNAs for saCas9 and 6 sgRNA (selected from 97 total designed sgRNAs) for SluCas9 were selected to be screened to excise the E-boxes (sgRNAs are designed within the range of +/−150 bp from the first or last Ebox).

FIG. 9 shows the experiment design and workflow chart for SaCas9 and SluCas9 in vitro study of E-box excision in DM1 patient primary myoblasts.

FIG. 10A-B show PCR and TapeStation verification of paired sgRNAs-induced Ebox excision in DMSO treated DM1 patient myoblasts. FIG. 10A shows how excision was verified by running TapeStation of PCR amplification of genomic DNA prepared from DMSO treated DM1 patient myoblasts 72 h post nucleofection (G1 in FIG. 9). For both SaCas9 and sluCas9 sgRNA pairs, comparing to the band results from the Mock and healthy control (marked by arrow), most of them showed the ability to induce smaller amplicon bands indicative of excision evens. FIG. 10B shows ICE analysis of Sanger sequencing of PCR amplification of genomic DNA prepared from DMSO treated DM1 patient myoblasts 72 h post nucleofection (G1 in FIG. 9). * indicate the sample contains ICE analysis results with low R2 values.

FIG. 11A-B show PCR and TapeStation verification of paired sgRNAs-induced Ebox excision in DNA-Pki treated DM1 patient myoblasts. FIG. 11A shows how excision was verified by running TapeStation of PCR amplification of genomic DNA prepared from DNA-Pki (3 μM) treated DM1 patient myoblasts 72 h post nucleofection (G4 in FIG. 9). For both SaCas9 and sluCas9 sgRNA pairs, comparing to the size of amplicon of the Mock and healthy control (marked arrow line), most of them showed the ability to induce smaller amplicon bands indicative of excision evens. FIG. 10B shows ICE analysis of Sanger sequencing of PCR amplification of genomic DNA prepared from DNA-Pki (3 μM) treated DM1 patient myoblasts 72 h post nucleofection (G4 in FIG. 9). NA indicate the sample failed ICE analysis and * indicate the sample with low R2 values.

FIG. 12A-B show the reduction of DMPK transcript level by E-boxes excision in DM1 patient myotubes. DMPK transcript level was quantified by ddPCR and presented as mean±SD (n=2-3). All the samples were treated with DMSO (FIG. 12A) or 3 μM of DNA-PKi (FIG. 12B) for 48 h after RNP delivery.

FIG. 13 shows CUG foci number reduction induced by E-box targeted gene editing by saCas9 or sluCas9 with/without DNA-pki treatment in primary DM1 patient myotubes. The data are presented as mean±SD (n=2-3). The dotted line indicates the average CUG foci number per myonuclei in Mock treated DM1 primary patient myotubes (DMSO). DM1 Mock served as a negative control (dashed line: DMSO treated DM1 mock), and primary Healthy myoblasts served as a positive control. sluU64D34 (solid line: DMSO sluU64-CTG/D34-CTG) was used as a positive control to compare efficiency of average Foci number reduction between CTG repeat excision and the Ebox cis-element excision. Certain saCas9 and sluCas9 pairs were able to achieve the level of foci reduction like the CTG excision pair (SluU64-CTG/D34-CTG) supporting the cis-element small fragment excision strategy can induce foci reduction to a similar level as to directly large fragment CTG repeat excision. The myotube samples were treated with DNA-PKi for 48 h after RNP nucleofection (open bars) or without DNA-PKi (Gray bars).

FIG. 14A-B shows an overview of single clone development. FIG. 14A shows a schematic view of five specific deletions of cis-element region of DMPK gene in healthy immortalized human myoblast line. FIG. 14B shows a summary of clones derived from single cells with different deletion genotype.

FIG. 15A-C shows a characterization of single cell derived clones with different deletion of cis-elements in DMPK gene. FIG. 15A shows a quantification of myogenic differentiation efficiency. FIGS. 15B and 15C show the quantification of relative DMPK mRNA expression level in clones with different deletion profile and percentage of decrease compare with the level in non-deletion clones.

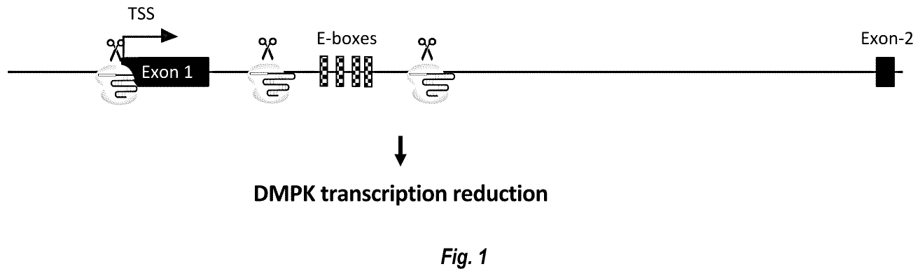

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measureable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide,” “nucleic acid,” and “nucleic acid molecule,” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O6-methylguanine, 4-thio-pyrimidines, 4-amino-pyrimidines, 4-dimethylhydrazine-pyrimidines, and 04-alkyl-pyrimidines; U.S. Pat. No. 5,378,825 and PCT No. WO 93/13121). For general discussion see The Biochemistry of the Nucleic Acids 5-36, Adams et al., ed., 11th ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “guide RNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “guide RNA” refers to each type. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas protein (e.g., Cas9). A guide sequence can be 20 base pairs in length, e.g., in the case of Streptococcus pyogenes (i.e., Spy Cas9, SpCas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 17, 18, 19, or 20 contiguous nucleotides of a sequence selected from SEQ ID NOs: 1-12. In preferred embodiments, a guide/spacer sequence in the case of SluCas9 or SaCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). In some embodiments, the guide sequence comprises a sequence selected from SEQ ID NOs: 1-12. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 17, 18, 19, or 20 contiguous nucleotides of a sequence selected from SEQ ID NOs: 1-12. In some embodiments, the guide sequence comprises a sequence with about 75%, 80%, 85%, 90%, 95%, 96%, 97%, 98%, 99%, or 100% identity to a sequence selected from SEQ ID NOs: 1-12. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides. In general, as set forth above, the U residues in any of the RNA sequences described herein may be replaced with T residues when describing a DNA sequence encoding the RNA.

In some embodiments, the guide sequence comprises a sequence selected from SEQ ID NOs: 1-12, 1300-1308, and 1400-1496 wherein if the 5′ terminal nucleotide is not guanine, one or more guanine (g) is added to the sequence at its 5′ end. The 5′ g or gg is required in some instances for transcription, for example, for expression by the RNA polymerase III-dependent U6 promoter or the T7 promoter. In some embodiments, a 5′ guanine is added to any one of the guide sequences or pairs of guide sequences disclosed herein.

Target sequences for Cas proteins, including Cas9s, include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “ribonucleoprotein” (RNP) or “RNP complex” refers to a guide RNA together with an RNA-targeted endonuclease, which can include a Cas protein such as Cas9. In some embodiments, the guide RNA guides the, e.g., Cas9 (such as SpCas9, SaCas9, or SluCas9) to a target sequence, and the guide RNA hybridizes with and the agent binds to the target sequence, which can be followed by cleaving or nicking (in the context of a modified “nickase” Cas9).

As used herein, an “RNA-targeted endonuclease” means a polypeptide or complex of polypeptides having RNA and DNA binding activity and DNA cleavage activity, or a DNA-binding subunit of such a complex, wherein the DNA binding activity is sequence-specific and depends on the sequence of the RNA. Exemplary RNA-targeted endonucleases include Cas cleavases/nickases. “Cas nuclease”, also called “Cas protein” as used herein, encompasses Cas cleavases and Cas nickases. Cas cleavases/nickases include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. In some embodiments, the Cas protein is not a Cas nickase. In some embodiments, the Cas protein is not a Cas nickase and CRISPR interference is not used. In particular embodiments, the Cas protein is not a nuclease-deficient/nuclease-dead Cas protein.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, a “toxic protein” is a protein that, following expression of the protein by a cell, causes deleterious effects (e.g., death or functional impairment) to the cell or to one or more surrounding cells. In some embodiments, the toxic protein is a misfolded protein. In particular embodiments, the toxic protein is a mutant gain-of-function protein. In some embodiments, the toxic protein comprises an amino acid repeat region (e.g., a polyglutamine repeat region) that is longer than found in the corresponding wildtype protein expressed by a healthy control cell. In some embodiments, the amino acid repeat region is at least one, two, five, eight, ten, fifteen, twenty, twenty-five, thirty, forty-five, or fifty amino acids longer than the amino acid repeat region in the corresponding wildtype protein expressed by a healthy control cell. In some embodiments, the amino acid repeat region is 1-3, 1-5, 5-10, 5-15, 5-25, 5-50, 10-25, 10-50, 20-30, or 25-50 amino acids longer than the amino acid repeat region in the corresponding wildtype protein expressed by a healthy control cell. In some embodiments, if the toxic protein is Huntingtin protein, the protein comprises more than 36, or more than 40 polyglutamine repeats. In some embodiments, the amino acid repeat region is 5%, 15%, 20%, 25%, 50%, 75%, or 100% longer in the toxic protein as compared to the amino acid repeat region in the corresponding wildtype protein expressed by a healthy control cell. In some embodiments, the amino acid repeat region is 5-10%, 5-25%, 5-50%, 5-75%, 5-100%, 20-50%, 20-75%, 20-100%, 50-75%, 50-100%, or 75-100%, longer in the toxic protein as compared to the amino acid repeat region in the corresponding wildtype protein expressed by a healthy control cell. Examples of toxic proteins include proteins produced from any one or more of the following genes: DM1 protein kinase (DMPK), CCHC-type zinc finger nucleic acid binding protein (CNBP), transcription Factor 4 (TCF4), junctophilin 3 (JPH3), huntingtin (HTT), ataxin 1 (ATXN1), ataxin 2 (ATXN2), ataxin 3 (ATXN3), ataxin 7 (ATXN7), ATXN8 opposite strand lncRNA (ATXN8OS), androgen receptor (AR), atrophin 1 (ATN1), fragile X Mental Retardation 1 (FMR1), chromosome 9 open reading frame 72 (C90RF72), polyadenylate-binding protein 2 (PABP2), amyloid precursor protein (APP), presenilin 1 (PSEN1), presenilin 2 (PSEN2), apolipoprotein E4 (APOE4), and synuclein alpha (SNCA).

As used herein, a toxic transcript is an RNA transcript that, following transcription of the transcript by a cell, causes deleterious effects (e.g., death or functional impairment) to the cell or to one or more surrounding cells. In particular embodiments, the toxic transcript is a mutant gain-of-function transcript. In particular embodiments, the toxic transcript comprises a larger number of trinucleotide repeats as compared to the corresponding wildtype transcript in a healthy control cell. In some embodiments, the toxic transcript comprises at least 50, 100, 200, 300, 400, 500, 600, 700, 800, 900, 1000, 1200, 1500 more trinucleotide repeats as compared to the corresponding wildtype transcript in a healthy control cell. In some embodiments, the toxic transcript comprises 50-1500, 300-1500, 700-1500, 1000-1500, 1200-1500, 50-1000, 300-1000, 700-1000, 50-700, or 300-700 more trinucleotide repeats as compared to the corresponding wildtype transcript in a healthy control cell. In some embodiments, the toxic transcript comprises at least 5%, 10%, 15%, 20%, or 25%, 50%, 75%, 100%, or 125% more trinucleotide repeats than the corresponding wildtype transcript in a healthy control cell. In some embodiments, the toxic transcript comprises at least 5-125%, 25-125%, 50-125%, 75-125%, 100-125%, 5-100%, 25-100%, 50-100%, 75-100%, 5-75%, 5-50%, or 25-50%, 5-25% more trinucleotide repeats than the corresponding wildtype transcript in a healthy control cell. In some embodiments, the toxic transcript impairs splicing machinery in the cell. In some embodiments, the toxic transcript is a mutant DMPK transcript.

Guide sequences useful in the guide RNA compositions and methods described herein are shown in Tables 3A-B, 5A-C, and 6A-B and throughout the application.

As used herein, a “target sequence” refers to a sequence of nucleic acid in a target gene that has complementarity to at least a portion of the guide sequence of the guide RNA. The interaction of the target sequence and the guide sequence directs a Cas protein (e.g., Cas9) to bind, and potentially nick or cleave (depending on the activity of the agent), within the target sequence. In some embodiments, the target sequences is “near” a regulatory element. In some embodiments, a target sequence is within 5, 10, 20, 30, 40, 50 or 100 nucleotides of a regulatory element, where the distance from the target to the regulatory element is measured as the number of nucleotides between the closest nucleotide of the regulatory element and the site that undergoes cleavage. In some embodiments, a target sequence is within 5-10, 5-20, 5-40, 5-60, 5-80, 5-100, 10-20, 10-40, 10-60, 10-80, 10-100, 20-40, 20-60, 20-80, 20-100, 40-60, 40-80, 40-100, 60-80, 60-100, or 80-100 nucleotides of a regulatory element. In some embodiments, a target sequence is within 100, 200, 300, 400, 500, 600, 700, 800, 900, 1000, 1100, 1200, 1300, 1400 or 1500 nucleotides of a regulatory element. In some embodiments, the target sequence is within 100-1000, 100-800, 100-600, 100-400, 100-200, 300-1000, 300-800, 300-600, 500-1000, or 500-800 nucleotides of a regulatory element. In some embodiments two guide sequences flank a regulatory element or a portion of a regulatory element. In some embodiments, the flanking guide sequences excise the regulatory element or a portion of the regulatory element. In some embodiments, the guide sequences do not flank a regulatory element, but nevertheless disrupt the regulatory element so that the gene that the regulatory element regulates produces less transcript than normal.

In some embodiments, the disclosure provides for a pair of guide RNAs (e.g., a pair of any of the guide RNAs disclosed herein) that, when combined with an appropriate endonuclease (e.g., any of the Cas proteins disclosed herein), is capable of excising a target region (e.g., at least a portion of a regulatory element) of a nucleic acid. In some embodiments, the excised portion is less than 3000, 2500, 2200, 2000, 1800, 1500, 1400, 1300, 1200, 1100, 1000, 900, 800, 700, 600, 500, 400, 300, 200, 100, 50, or 25 nucleotides in length. In particular embodiments, the excised portion is less than 800 nucleotides in length. In other embodiments, the excised portion is less than 300 nucleotides in length. In further embodiments, the excised portion is less than 100 nucleotides in length. In some embodiments, the excised portion is between 25-2000, 25-1500, 25-1000, 25-700, 25-400, 25-250, 25-100, 100-2000, 100-1500, 100-1000, 100-700, 100-400, 100-250, 250-2000, 250-1500, 250-1000, 250-700, 250-400, 400-2000, 400-1500, 400-1000, 400-700, 700-2000, 700-1500, 700-1000, 1000-2000, 1000-1500, or 1500-2000 nucleotides in length. In particular embodiments, the excised portion is between 25-800 nucleotides in length. In more particular embodiments, the excised portion is between 25-400 nucleotides in length. In more particular embodiments, the excised portion is between 50-200 nucleotides in length.

In some embodiments, the target sequence is near or flanks a transcriptional regulatory element of a target gene. In some embodiments, the regulatory element is in an intron. In some embodiments, where the regulatory element is in an intron, the target sequences are in the same intron. In some embodiments, the target is a transcription start (TSS) site. In some embodiments, the target is an intronic E-box site. In some embodiments, the target is a transcription start (TSS) site and four intronic E-box site. In some embodiments, the target for excision includes an exon of a gene. In some embodiments, the target is intron 1 and exon 1 of a gene. In some embodiments, the target is intron 1 and exon 1 of the DMPK gene. In some embodiments, excision of the target region (e.g., one or more introns, exons, and/or regulatory regions) results in a cell expressing a lower-expression splice form of the gene. For example, in some embodiments, excision of all or most of intron 1 and exon 1 of the DMPK gene from DNA in a muscle cell will result in expression of a DMPK splice form that is expressed at lower levels in the muscle cell than a DMPK splice form including exon 1. In some embodiments, the target region for excision includes exon 1 of the DMPK gene. In some embodiments, the exon encodes a portion of one, but not all, isoforms of a gene. In some embodiments, such as, for example, where the HTT gene is implicated (Huntington Disease), the regulatory element can be a cis-regulatory element for regulating gene expression, including NF-κB binding domain, 20 bp direct repeats, AP2, Sp1, HDBP1/2, or UCRR. (Thomsona and Leavitta, J Huntingtons Dis. 2018; PMID: 30452421). In some embodiments, such as, for example, where the FMR1 gene is implicated (Fragile X-associated tremor/ataxia syndrome), the regulatory element can be a cis-regulatory element known as 22-bp methylation sensitive element (MSE). (Hwu et al., DNA Cell Biol. 1997; PMID: 9150432). In addition, the single-nucleotide polymorphism (SNP) variant T/C (ss71651738) may promote repeat expansion and may be targeted in the methods described herein. (Gerhardt et al., J Cell Biol. 2014; PMID: 25179629).

As used herein, “treatment” when used in the context of a disease or disorder refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. Treatment of any disease or disorder characterized by toxic proteins and transcripts, such as, for example, a disease or disorder caused by a trinucleotide repeat (TNR), may comprises alleviating the symptoms of the disease or disorder. For example, treatment of DM1 may comprise alleviating symptoms of DM1. Similarly, treatment of, e.g., Myotonic dystrophy type 2 (DM2), Fuchs endothelial corneal dystrophy (FECD), Huntington disease-like 2 (HDL2), Huntington disease (HD), Spinocerebellar ataxia (SCA), Spinal and bulbar muscular atrophy (SBMA), Dentatorubral-pallidoluysian atrophy (DRPLA), Fragile X-associated tremor/ataxia syndrome (FXTAS), Frontotemporal dementia (FTD), amyotrophic lateral sclerosis (ALS), Oculopharyngeal muscular dystrophy (OPMD), Alzheimer's Disease (AD), and Parkinson's Disease (PD) may comprise alleviating the symptoms of those diseases.

As used herein, “ameliorating” refers to any beneficial effect on a phenotype or symptom, such as reducing its severity, slowing or delaying its development, arresting its development, or partially or completely reversing or eliminating it. In the case of quantitative phenotypes such as expression levels, ameliorating encompasses changing the expression level so that it is closer to the expression level seen in healthy or unaffected cells or individuals.

As used herein, “DM1 myoblasts” refer to precursors of muscle cells that have a genotype associated with DM1, and include e.g., cells derived from or isolated from a subject with DM1. DM1 myoblasts include primary cells, cultured cells, or cell lines.

A “pharmaceutically acceptable excipient” refers to an agent that is included in a pharmaceutical formulation that is not the active ingredient. Pharmaceutically acceptable excipients may e.g., aid in drug delivery or support or enhance stability or bioavailability.

The term “about” or “approximately” means an acceptable error for a particular value as determined by one of ordinary skill in the art, which depends in part on how the value is measured or determined.

### II. Compositions

Provided herein are compositions comprising one or more of any of the guide RNAs disclosed herein. In some embodiments, the disclosure provides for a composition comprising any one or more of the guides disclosed in Tables, 3A, 3B, 5A, 5B, 5C, 6A or 6B. In some embodiments, the composition comprises a guide sequence selected from any one of SEQ ID Nos: 1-11. In some embodiments, the composition comprises a guide sequence selected from any one of SEQ ID Nos: 1-11 and an endonuclease (e.g., SpCas9) or a nucleic acid encoding an endonuclease (e.g., SpCas9). In some embodiments, the composition comprises a guide sequence selected from any one of SEQ ID Nos: 1300-1308. In some embodiments, the composition comprises a guide sequence selected from any one of SEQ ID Nos: 1300-1308 and an endonuclease (e.g., SaCas9) or a nucleic acid encoding an endonuclease (e.g., SaCas9). In some embodiments, the composition comprises a guide sequence selected from any one of SEQ ID Nos: 1400-1496. In some embodiments, the composition comprises a guide sequence selected from any one of SEQ ID Nos: 1400-1496 and an endonuclease (e.g., SluCas9) or a nucleic acid encoding an endonuclease (e.g., SluCas9). In some embodiments, the composition comprises a guide sequence selected from any one of SEQ ID Nos: 1420, 1428, 1475, 1402, 1411, and 1463. In some embodiments, the composition comprises a guide sequence selected from any one of SEQ ID Nos: 1420, 1428, 1475, 1402, 1411, and 1463 and an endonuclease (e.g., SluCas9) or a nucleic acid encoding an endonuclease (e.g., SluCas9).

In some embodiments, the disclosure provides for a composition comprising a pair of guide RNAs targeting regulatory elements. In some embodiments, the pair of guide RNAs fully or partially excise the regulatory element that at least partially drives expression of a gene. In some embodiments, the pair of guide RNAs comprise a first guide comprising a first guide sequence selected from any one of SEQ ID Nos: 1-3 and a second guide comprising a second guide sequence selected from any one of SEQ ID Nos: 4-6. In some embodiments, the pair of guide RNAs comprise a first guide comprising a first guide sequence selected from any one of SEQ ID Nos: 7-9 and a second guide comprising a second guide sequence selected from any one of SEQ ID Nos: 10-11. In some embodiments, guide pairs comprising any of SEQ ID NOs: 1-6 are for use with SpCas9.

In some embodiments, the pair of guide RNAs comprise a first guide comprising a first guide sequence selected from any one of SEQ ID Nos: 1300, 1301, 1302, 1303, and 1304 and a second guide comprising a second guide sequence selected from any one of SEQ ID Nos: 1305, 1306, 1307, and 1308. In some embodiments, guide pairs comprising any of SEQ ID NOs: 1300-1308 are for use with SaCas9.

In some embodiments, the pair of guide RNAs comprise a first sequence selected from SEQ ID Nos: 1420, 1428, and 1475 and a second sequence selected from any one of SEQ ID Nos: 1402, 1411, and 1463. In some embodiments, guide pairs comprising any of SEQ ID NOs: 1420, 1428, 1475, 1402, 1411, and 1463 are for use with SluCas9.

Nucleic acids encoding such guide RNA pairs are encompassed. Such compositions may be administered/delivered to subjects having or suspected of having a disease or disorder, e.g., characterized by toxic proteins and transcripts.

In some embodiments, a composition is provided comprising a pair of guide RNAs, or nucleic acids encoding same, targeting a TSS of the DMPK gene, wherein the pair of guide RNAs comprise a first guide RNA comprising at least 17, 18, 19, or 20 contiguous nucleotides of a first guide sequence selected from any one of SEQ ID NOs: 1, 2, and 3, and a second guide RNA comprising at least 17, 18, 19, 20, or 21 contiguous nucleotides of a second guide sequence selected from any one of SEQ ID NOs: 4, 5, and 6. In some embodiments, the pair of guide RNAs comprises:
a. a first guide RNA comprising at least 17, 18, 19, or 20 contiguous nucleotides of a first guide sequence of SEQ ID NO: 1 and a second guide RNA comprising at least 17, 18, 19, 20, or 21 contiguous nucleotides of a second guide sequence of SEQ ID NO: 4;
b. a first guide RNA comprising at least 17, 18, 19, or 20 contiguous nucleotides of a first guide sequence of SEQ ID NO: 1 and a second guide RNA comprising at least 17, 18, 19, 20, or 21 contiguous nucleotides of a second guide sequence of SEQ ID NO: 5;
c. a first guide RNA comprising at least 17, 18, 19, or 20 contiguous nucleotides of a first guide sequence of SEQ ID NO: 1 and a second guide RNA comprising at least 17, 18, 19, 20, or 21 contiguous nucleotides of a second guide sequence of SEQ ID NO: 6;
d. a first guide RNA comprising at least 17, 18, 19, or 20 contiguous nucleotides of a first guide sequence of SEQ ID NO: 2 and a second guide RNA comprising at least 17, 18, 19, 20, or 21 contiguous nucleotides of a second guide sequence of SEQ ID NO: 4;
e. a first guide RNA comprising at least 17, 18, 19, or 20 contiguous nucleotides of a first guide sequence of SEQ ID NO: 2 and a second guide RNA comprising at least 17, 18, 19, 20, or 21 contiguous nucleotides of a second guide sequence of SEQ ID NO: 5;
f. a first guide RNA comprising at least 17, 18, 19, or 20 contiguous nucleotides of a first guide sequence of SEQ ID NO: 2 and a second guide RNA comprising at least 17, 18, 19, 20, or 21 contiguous nucleotides of a second guide sequence of SEQ ID NO: 6;
g. a first guide RNA comprising at least 17, 18, 19, or 20 contiguous nucleotides of a first guide sequence of SEQ ID NO: 3 and a second guide RNA comprising at least 17, 18, 19, 20, or 21 contiguous nucleotides of a second guide sequence of SEQ ID NO: 4;
h. a first guide RNA comprising at least 17, 18, 19, or 20 contiguous nucleotides of a first guide sequence of SEQ ID NO: 3 and a second guide RNA comprising at least 17, 18, 19, 20, or 21 contiguous nucleotides of a second guide sequence of SEQ ID NO: 5; or
i. a first guide RNA comprising at least 17, 18, 19, or 20 contiguous nucleotides of a first guide sequence of SEQ ID NO:3 and a second guide RNA comprising at least 17, 18, 19, 20, or 21 contiguous nucleotides of a second guide sequence of SEQ ID NO: 6.

In some embodiments, a composition is provided comprising a pair of guide RNAs, or nucleic acids encoding same, targeting a TSS of the DMPK gene, wherein the pair of guide RNAs comprise a first guide RNA comprising a first guide sequence selected from any one of SEQ ID NOs: 1, 101, 102, 103, 2, 201, 202, 203, 3, 301, 302, and 303; and a second guide RNA comprising a second guide sequence selected from any one of SEQ ID NOs: 4, 401, 402, 403, 5, 501, 502, 503, 6, 601, 602, and 603. In some embodiments, the first guide RNA comprises a first guide sequence selected from any one of SEQ ID NOs: 1, 2, and 3; and the second guide RNA comprises a second guide sequence selected from any one of SEQ ID NOs: 4, 5, and 6. In some embodiments, the first guide RNA comprises a first guide sequence selected from any one of SEQ ID NOs: 101, 201, and 301; and the second guide RNA comprises a second guide sequence selected from any one of SEQ ID NOs: 401, 501, and 601. In some embodiments, the first guide RNA comprises a first guide sequence selected from any one of SEQ ID NOs: 102, 202, and 302; and the second guide RNA comprises a second guide sequence selected from any one of SEQ ID NOs: 402, 502, and 602. In some embodiments, the first guide RNA comprises a first guide sequence selected from any one of SEQ ID NOs: 103, 203, and 303; and the second guide RNA comprises a second guide sequence selected from any one of SEQ ID NOs: 403, 503, and 603. Nucleic acids encoding any of the above guide RNA pairs is encompassed.

In some embodiments, a composition is provided comprising a pair of guide RNAs targeting a TSS of the DMPK gene, wherein the pair of guide RNAs comprise a first guide RNA comprising a first guide sequence that binds to a nucleotide sequence that is complementary to any one of SEQ ID NOs: 1, 2, and 3, and a second guide RNA comprising a second guide sequences that binds to a nucleotide sequence that is complementary to any one of SEQ ID NOs: 4, 5, and 6. In some embodiments, the pair of guide RNAs comprises:
a. a first guide RNA comprising a guide sequence that binds to a nucleotide sequence that is complementary to SEQ ID NO: 1 and a second guide RNA comprising a guide sequence that binds to a nucleotide sequence that is complementary to SEQ ID NO: 4;
b. a first guide RNA comprising a guide sequence that binds to a nucleotide sequence that is complementary to SEQ ID NO: 1 and a second guide RNA comprising a guide sequence that binds to a nucleotide sequence that is complementary to SEQ ID NO: 5;
c. a first guide RNA comprising a guide sequence that binds to a nucleotide sequence that is complementary to SEQ ID NO: 1 and a second guide RNA comprising a guide sequence that binds to a nucleotide sequence that is complementary to SEQ ID NO: 6;
d. a first guide RNA comprising a guide sequence that binds to a nucleotide sequence that is complementary to SEQ ID NO: 2 and a second guide RNA comprising a guide sequence that binds to a nucleotide sequence that is complementary to SEQ ID NO: 4;
e. a first guide RNA comprising a guide sequence that binds to a nucleotide sequence that is complementary to SEQ ID NO: 2 and a second guide RNA comprising a guide sequence that binds to a nucleotide sequence that is complementary to SEQ ID NO: 5;
f. a first guide RNA comprising a guide sequence that binds to a nucleotide sequence that is complementary to SEQ ID NO: 2 and a second guide RNA comprising a guide sequence that binds to a nucleotide sequence that is complementary to SEQ ID NO: 6;
g. a first guide RNA comprising a guide sequence that binds to a nucleotide sequence that is complementary to SEQ ID NO: 3 and a second guide RNA comprising a guide sequence that binds to a nucleotide sequence that is complementary to SEQ ID NO: 4;
h. a first guide RNA comprising a guide sequence that binds to a nucleotide sequence that is complementary to SEQ ID NO: 3 and a second guide RNA comprising a guide sequence that binds to a nucleotide sequence that is complementary to SEQ ID NO: 5; or
i. a first guide RNA comprising a guide sequence that binds to a nucleotide sequence that is complementary to SEQ ID NO: 3 and a second guide RNA comprising a guide sequence that binds to a nucleotide sequence that is complementary to SEQ ID NO: 6.

Nucleic acids encoding any of the above guide RNA pairs is encompassed.

In some embodiments, a composition is provided comprising a pair of guide RNAs, or nucleic acids encoding same, targeting an E-box of the DMPK gene, wherein the pair of guide RNAs comprise a first guide RNA comprising at least 17, 18, 19, or 20 contiguous nucleotides of a first guide sequence selected from any one of SEQ ID NOs: 7, 8, and 9, and a second guide RNA comprising at least 17, 18, 19, or 20 contiguous nucleotides of a second guide sequence selected from any one of SEQ ID NOs: 10, 11, and 12. In some embodiments, the pair of guide RNAs comprises:
a. a first guide RNA comprising at least 17, 18, 19, or 20 contiguous nucleotides of a first guide sequence of SEQ ID NO: 7 and a second guide RNA comprising at least 17, 18, 19, or 20 contiguous nucleotides of a second guide sequence of SEQ ID NO: 10;
b. a first guide RNA comprising at least 17, 18, 19, or 20 contiguous nucleotides of a first guide sequence of SEQ ID NO: 7 and a second guide RNA comprising at least 17, 18, 19, or 20 contiguous nucleotides of a second guide sequence of SEQ ID NO: 11;
c. a first guide RNA comprising at least 17, 18, 19, or 20 contiguous nucleotides of a first guide sequence of SEQ ID NO: 7 and a second guide RNA comprising at least 17, 18, 19, or 20 contiguous nucleotides of a second guide sequence of SEQ ID NO: 12;
d. a first guide RNA comprising at least 17, 18, 19, or 20 contiguous nucleotides of a first guide sequence of SEQ ID NO: 8 and a second guide RNA comprising at least 17, 18, 19, or 20 contiguous nucleotides of a second guide sequence of SEQ ID NO: 10;
e. a first guide RNA comprising at least 17, 18, 19, or 20 contiguous nucleotides of a first guide sequence of SEQ ID NO: 8 and a second guide RNA comprising at least 17, 18, 19, or 20 contiguous nucleotides of a second guide sequence of SEQ ID NO: 11;
f. a first guide RNA comprising at least 17, 18, 19, or 20 contiguous nucleotides of a first guide sequence of SEQ ID NO: 8 and a second guide RNA comprising at least 17, 18, 19, or 20 contiguous nucleotides of a second guide sequence of SEQ ID NO: 12;
g. a first guide RNA comprising at least 17, 18, 19, or 20 contiguous nucleotides of a first guide sequence of SEQ ID NO: 9 and a second guide RNA comprising at least 17, 18, 19, or 20 contiguous nucleotides of a second guide sequence of SEQ ID NO: 10;
h. a first guide RNA comprising at least 17, 18, 19, or 20 contiguous nucleotides of a first guide sequence of SEQ ID NO: 9 and a second guide RNA comprising at least 17, 18, 19, or 20 contiguous nucleotides of a second guide sequence of SEQ ID NO: 11; or
i. a first guide RNA comprising at least 17, 18, 19, or 20 contiguous nucleotides of a first guide sequence of SEQ ID NO: 9 and a second guide RNA comprising at least 17, 18, 19, or 20 contiguous nucleotides of a second guide sequence of SEQ ID NO: 12.

Nucleic acids encoding any of the above guide RNA pairs is encompassed.

In some embodiments, a composition is provided comprising a pair of guide RNAs targeting an E-box of the DMPK gene, wherein the pair of guide RNAs comprise a first guide RNA comprising at least 17, 18, 19, or 20 contiguous nucleotides of a first guide sequence selected from any one of SEQ ID NOs: 1300, 1301, 1302, 1303, and 1304, and a second guide RNA comprising at least 17, 18, 19, or 20 contiguous nucleotides of a second guide sequence selected from any one of SEQ ID NOs: 1305, 1306, 1307, and 1308. In some embodiments, the pair of guide RNAs comprises:
a. a first guide RNA comprising at least 17, 18, 19, 20, 21, or 22 contiguous nucleotides of a first guide sequence of SEQ ID NO: 1300 and a second guide RNA comprising at least 17, 18, 19, 20, 21, or 22 contiguous nucleotides of a second guide sequence of SEQ ID NO: 1305;
b. a first guide RNA comprising at least 17, 18, 19, 20, 21, or 22 contiguous nucleotides of a first guide sequence of SEQ ID NO: 1300 and a second guide RNA comprising at least 17, 18, 19, 20, 21, or 22 contiguous nucleotides of a second guide sequence of SEQ ID NO: 1306;
c. a first guide RNA comprising at least 17, 18, 19, 20, 21, or 22 contiguous nucleotides of a first guide sequence of SEQ ID NO: 1300 and a second guide RNA comprising at least 17, 18, 19, 20, 21, or 22 contiguous nucleotides of a second guide sequence of SEQ ID NO: 1307;
d. a first guide RNA comprising at least 17, 18, 19, 20, 21, or 22 contiguous nucleotides of a first guide sequence of SEQ ID NO: 1300 and a second guide RNA comprising at least 17, 18, 19, 20, 21, or 22 contiguous nucleotides of a second guide sequence of SEQ ID NO: 1308;
e. a first guide RNA comprising at least 17, 18, 19, 20, 21, or 22 contiguous nucleotides of a first guide sequence of SEQ ID NO: 1301 and a second guide RNA comprising at least 17, 18, 19, 20, 21, or 22 contiguous nucleotides of a second guide sequence of SEQ ID NO: 1305;
f. a first guide RNA comprising at least 17, 18, 19, 20, 21, or 22 contiguous nucleotides of a first guide sequence of SEQ ID NO: 1301 and a second guide RNA comprising at least 17, 18, 19, 20, 21, or 22 contiguous nucleotides of a second guide sequence of SEQ ID NO: 1306;
g. a first guide RNA comprising at least 17, 18, 19, 20, 21, or 22 contiguous nucleotides of a first guide sequence of SEQ ID NO: 1301 and a second guide RNA comprising at least 17, 18, 19, 20, 21, or 22 contiguous nucleotides of a second guide sequence of SEQ ID NO: 1307;
h. a first guide RNA comprising at least 17, 18, 19, 20, 21, or 22 contiguous nucleotides of a first guide sequence of SEQ ID NO: 1301 and a second guide RNA comprising at least 17, 18, 19, 20, 21, or 22 contiguous nucleotides of a second guide sequence of SEQ ID NO: 1308;
i. a first guide RNA comprising at least 17, 18, 19, 20, 21, or 22 contiguous nucleotides of a first guide sequence of SEQ ID NO: 1302 and a second guide RNA comprising at least 17, 18, 19, 20, 21, or 22 contiguous nucleotides of a second guide sequence of SEQ ID NO: 1305;
j. a first guide RNA comprising at least 17, 18, 19, 20, 21, or 22 contiguous nucleotides of a first guide sequence of SEQ ID NO: 1302 and a second guide RNA comprising at least 17, 18, 19, 20, 21, or 22 contiguous nucleotides of a second guide sequence of SEQ ID NO: 1306;
k. a first guide RNA comprising at least 17, 18, 19, 20, 21, or 22 contiguous nucleotides of a first guide sequence of SEQ ID NO: 1302 and a second guide RNA comprising at least 17, 18, 19, 20, 21, or 22 contiguous nucleotides of a second guide sequence of SEQ ID NO: 1307;
l. a first guide RNA comprising at least 17, 18, 19, 20, 21, or 22 contiguous nucleotides of a first guide sequence of SEQ ID NO: 1302 and a second guide RNA comprising at least 17, 18, 19, 20, 21, or 22 contiguous nucleotides of a second guide sequence of SEQ ID NO: 1308;
m. a first guide RNA comprising at least 17, 18, 19, 20, 21, or 22 contiguous nucleotides of a first guide sequence of SEQ ID NO: 1303 and a second guide RNA comprising at least 17, 18, 19, 20, 21, or 22 contiguous nucleotides of a second guide sequence of SEQ ID NO: 1305;
n. a first guide RNA comprising at least 17, 18, 19, 20, 21, or 22 contiguous nucleotides of a first guide sequence of SEQ ID NO: 1303 and a second guide RNA comprising at least 17, 18, 19, 20, 21, or 22 contiguous nucleotides of a second guide sequence of SEQ ID NO: 1306;
o. a first guide RNA comprising at least 17, 18, 19, 20, 21, or 22 contiguous nucleotides of a first guide sequence of SEQ ID NO: 1303 and a second guide RNA comprising at least 17, 18, 19, 20, 21, or 22 contiguous nucleotides of a second guide sequence of SEQ ID NO: 1307;
p. a first guide RNA comprising at least 17, 18, 19, 20, 21, or 22 contiguous nucleotides of a first guide sequence of SEQ ID NO: 1303 and a second guide RNA comprising at least 17, 18, 19, 20, 21, or 22 contiguous nucleotides of a second guide sequence of SEQ ID NO: 1308;
q. a first guide RNA comprising at least 17, 18, 19, 20, 21, or 22 contiguous nucleotides of a first guide sequence of SEQ ID NO: 1304 and a second guide RNA comprising at least 17, 18, 19, 20, 21, or 22 contiguous nucleotides of a second guide sequence of SEQ ID NO: 1305;
r. a first guide RNA comprising at least 17, 18, 19, 20, 21, or 22 contiguous nucleotides of a first guide sequence of SEQ ID NO: 1304 and a second guide RNA comprising at least 17, 18, 19, 20, 21, or 22 contiguous nucleotides of a second guide sequence of SEQ ID NO: 1306;
s. a first guide RNA comprising at least 17, 18, 19, 20, 21, or 22 contiguous nucleotides of a first guide sequence of SEQ ID NO: 1304 and a second guide RNA comprising at least 17, 18, 19, 20, 21, or 22 contiguous nucleotides of a second guide sequence of SEQ ID NO: 1307; or
t. a first guide RNA comprising at least 17, 18, 19, 20, 21, or 22 contiguous nucleotides of a first guide sequence of SEQ ID NO: 1304 and a second guide RNA comprising at least 17, 18, 19, 20, 21, or 22 contiguous nucleotides of a second guide sequence of SEQ ID NO: 1308.

Nucleic acids encoding any of the above guide RNA pairs is encompassed.

In some embodiments, a composition is provided comprising a pair of guide RNAs, or nucleic acids encoding same, targeting an E-box of the DMPK gene, wherein the pair of guide RNAs comprise a first guide RNA comprising at least 17, 18, 19, 20, 21, or 22 contiguous nucleotides of a first guide sequence selected from any one of SEQ ID NOs: 1420, 1428, and 1475, and a second guide RNA comprising at least 17, 18, 19, and 20 contiguous nucleotides of a second guide sequence selected from any one of SEQ ID NOs: 1402, 1411, and 1463. In some embodiments, the pair of guide RNAs comprises:
a. a first guide RNA comprising at least 17, 18, 19, 20, 21, or 22 contiguous nucleotides of a first guide sequence of SEQ ID NO: 1420 and a second guide RNA comprising at least 17, 18, 19, 20, 21, or 22 contiguous nucleotides of a second guide sequence of SEQ ID NO: 1402;
b. a first guide RNA comprising at least 17, 18, 19, 20, 21, or 22 contiguous nucleotides of a first guide sequence of SEQ ID NO: 1420 and a second guide RNA comprising at least 17, 18, 19, 20, 21, or 22 contiguous nucleotides of a second guide sequence of SEQ ID NO: 1411;
c. a first guide RNA comprising at least 17, 18, 19, 20, 21, or 22 contiguous nucleotides of a first guide sequence of SEQ ID NO: 1420 and a second guide RNA comprising at least 17, 18, 19, 20, 21, or 22 contiguous nucleotides of a second guide sequence of SEQ ID NO: 1463;
d. a first guide RNA comprising at least 17, 18, 19, 20, 21, or 22 contiguous nucleotides of a first guide sequence of SEQ ID NO: 1428 and a second guide RNA comprising at least 17, 18, 19, 20, 21, or 22 contiguous nucleotides of a second guide sequence of SEQ ID NO: 1402;
e. a first guide RNA comprising at least 17, 18, 19, 20, 21, or 22 contiguous nucleotides of a first guide sequence of SEQ ID NO: 1428 and a second guide RNA comprising at least 17, 18, 19, 20, 21, or 22 contiguous nucleotides of a second guide sequence of SEQ ID NO: 1411;
f. a first guide RNA comprising at least 17, 18, 19, 20, 21, or 22 contiguous nucleotides of a first guide sequence of SEQ ID NO: 1428 and a second guide RNA comprising at least 17, 18, 19, 20, 21, or 22 contiguous nucleotides of a second guide sequence of SEQ ID NO: 1463;
g. a first guide RNA comprising at least 17, 18, 19, 20, 21, or 22 contiguous nucleotides of a first guide sequence of SEQ ID NO: 1475 and a second guide RNA comprising at least 17, 18, 19, 20, 21, or 22 contiguous nucleotides of a second guide sequence of SEQ ID NO: 1402;
h. a first guide RNA comprising at least 17, 18, 19, 20, 21, or 22 contiguous nucleotides of a first guide sequence of SEQ ID NO: 1475 and a second guide RNA comprising at least 17, 18, 19, 20, 21, or 22 contiguous nucleotides of a second guide sequence of SEQ ID NO: 1411; or
i. a first guide RNA comprising at least 17, 18, 19, 20, 21, or 22 contiguous nucleotides of a first guide sequence of SEQ ID NO: 1475 and a second guide RNA comprising at least 17, 18, 19, 20, 21, or 22 contiguous nucleotides of a second guide sequence of SEQ ID NO: 1463.

In some embodiments, a composition is provided comprising a pair of guide RNAs, or nucleic acids encoding same, targeting an E-box of the DMPK gene, wherein the pair of guide RNAs comprise a first guide RNA comprising a first guide sequence selected from any one of SEQ ID NOs: 7, 701, 702, 703, 8, 801, 802, 803, 9, 901, 902, and 903 and a second guide RNA comprising a second guide sequence selected from any one of SEQ ID NOs: 10, 1001, 1002, 1003, 11, 1101, 1102, 1103, 12, 1201, 1202, and 1203. In some embodiments, the first guide RNA comprises a first guide sequence selected from any one of SEQ ID NOs: 7, 8, and 9; and the second guide RNA comprises a second guide sequence selected from any one of SEQ ID NOs: 10, 11, and 12. In some embodiments, the first guide RNA comprises a first guide sequence selected from any one of SEQ ID NOs: 701, 801, and 901; and the second guide RNA comprises a second guide sequence selected from any one of SEQ ID NOs: 1001, 1101, and 1201. In some embodiments, the first guide RNA comprises a first guide sequence selected from any one of SEQ ID NOs: 702, 802, and 902; and the second guide RNA comprises a second guide sequence selected from any one of SEQ ID NOs: 1002, 1102, and 1202. In some embodiments, the first guide RNA comprises a first guide sequence selected from any one of SEQ ID NOs: 703, 803, and 903; and the second guide RNA comprises a second guide sequence selected from any one of SEQ ID NOs: 1003, 1103, and 1203.

In some embodiments, a composition is provided comprising a pair of guide RNAs, or nucleic acids encoding same, targeting an E-box of the DMPK gene, wherein the pair of guide RNAs comprise a first guide RNA comprising a first guide sequence selected from any one of SEQ ID NOs: 1300, 1301, 1302, 1303, and 1304 and a second guide RNA comprising a second guide sequence selected from any one of SEQ ID NOs: 1305, 1306, 1307, and 1308.

In some embodiments, a composition is provided comprising a pair of guide RNAs, or nucleic acids encoding same, targeting an E-box of the DMPK gene, wherein the pair of guide RNAs comprise a first guide sequence selected from any one of SEQ ID NOs: 1420, 1428, and 1475; and the second guide RNA comprises a second guide sequence selected from any one of SEQ ID NOs: 1402, 1411, and 1463.

In some embodiments, a composition is provided comprising a pair of guide RNAs, or nucleic acids encoding same, targeting a portion or the entirety of an E-box and a portion or the entirety of a TSS, optionally in the DMPK gene.

In some embodiments, a composition is provided comprising a pair of guide RNAs targeting an E-box of the DMPK gene, wherein the pair of guide RNAs comprise a first guide RNA comprising a first guide sequence that binds to a nucleotide sequence that is complementary to any one of SEQ ID NOs: 7, 8, or 9, and a second guide RNA comprising a second guide sequences that binds to a nucleotide sequence that is complementary to any one of SEQ ID NOs: 10, 11, or 12. In some embodiments, the pair of guide RNAs comprises:
a. a first guide RNA comprising a guide sequence that binds to a nucleotide sequence that is complementary to SEQ ID NO: 7 and a second guide RNA comprising a guide sequence that binds to a nucleotide sequence that is complementary to SEQ ID NO: 10;
b. a first guide RNA comprising a guide sequence that binds to a nucleotide sequence that is complementary to SEQ ID NO: 7 and a second guide RNA comprising a guide sequence that binds to a nucleotide sequence that is complementary to SEQ ID NO: 11;
c. a first guide RNA comprising a guide sequence that binds to a nucleotide sequence that is complementary to SEQ ID NO: 7 and a second guide RNA comprising a guide sequence that binds to a nucleotide sequence that is complementary to SEQ ID NO: 12;
d. a first guide RNA comprising a guide sequence that binds to a nucleotide sequence that is complementary to SEQ ID NO: 8 and a second guide RNA comprising a guide sequence that binds to a nucleotide sequence that is complementary to SEQ ID NO: 10;
e. a first guide RNA comprising a guide sequence that binds to a nucleotide sequence that is complementary to SEQ ID NO: 8 and a second guide RNA comprising a guide sequence that binds to a nucleotide sequence that is complementary to SEQ ID NO: 11;
f. a first guide RNA comprising a guide sequence that binds to a nucleotide sequence that is complementary to SEQ ID NO: 8 and a second guide RNA comprising a guide sequence that binds to a nucleotide sequence that is complementary to SEQ ID NO: 12;
g. a first guide RNA comprising a guide sequence that binds to a nucleotide sequence that is complementary to SEQ ID NO: 9 and a second guide RNA comprising a guide sequence that binds to a nucleotide sequence that is complementary to SEQ ID NO: 10;
h. a first guide RNA comprising a guide sequence that binds to a nucleotide sequence that is complementary to SEQ ID NO: 9 and a second guide RNA comprising a guide sequence that binds to a nucleotide sequence that is complementary to SEQ ID NO: 11; or
i. a first guide RNA comprising a guide sequence that binds to a nucleotide sequence that is complementary to SEQ ID NO: 9 and a second guide RNA comprising a guide sequence that binds to a nucleotide sequence that is complementary to SEQ ID NO: 12.

In some embodiments, a composition is provided comprising a pair of guide RNAs, or nucleic acids encoding same, targeting an E-box of the DMPK gene, wherein the pair of guide RNAs comprise a first guide RNA comprising a first guide sequence that binds to a nucleotide sequence that is complementary to any one of SEQ ID NOs: 1300, 1301, 1302, 1303, or 1304, and a second guide RNA comprising a second guide sequences that binds to a nucleotide sequence that is complementary to any one of SEQ ID NOs: 1305, 1306, 1307, or 1308. In some embodiments, the pair of guide RNAs comprises:
a. a first guide RNA comprising a guide sequence that binds to a nucleotide sequence that is complementary to SEQ ID NO: 1300 and a second guide RNA comprising a guide sequence that binds to a nucleotide sequence that is complementary to SEQ ID NO: 1305;
b. a first guide RNA comprising a guide sequence that binds to a nucleotide sequence that is complementary to SEQ ID NO: 1300 and a second guide RNA comprising a guide sequence that binds to a nucleotide sequence that is complementary to SEQ ID NO: 1306;
c. a first guide RNA comprising a guide sequence that binds to a nucleotide sequence that is complementary to SEQ ID NO: 1300 and a second guide RNA comprising a guide sequence that binds to a nucleotide sequence that is complementary to SEQ ID NO: 1307;
d. a first guide RNA comprising a guide sequence that binds to a nucleotide sequence that is complementary to SEQ ID NO: 1300 and a second guide RNA comprising a guide sequence that binds to a nucleotide sequence that is complementary to SEQ ID NO: 1308;
e. a first guide RNA comprising a guide sequence that binds to a nucleotide sequence that is complementary to SEQ ID NO: 1301 and a second guide RNA comprising a guide sequence that binds to a nucleotide sequence that is complementary to SEQ ID NO: 1305;
f. a first guide RNA comprising a guide sequence that binds to a nucleotide sequence that is complementary to SEQ ID NO: 1301 and a second guide RNA comprising a guide sequence that binds to a nucleotide sequence that is complementary to SEQ ID NO: 1306;
g. a first guide RNA comprising a guide sequence that binds to a nucleotide sequence that is complementary to SEQ ID NO: 1301 and a second guide RNA comprising a guide sequence that binds to a nucleotide sequence that is complementary to SEQ ID NO: 1307;
h. a first guide RNA comprising a guide sequence that binds to a nucleotide sequence that is complementary to SEQ ID NO: 1301 and a second guide RNA comprising a guide sequence that binds to a nucleotide sequence that is complementary to SEQ ID NO: 1308;
i. a first guide RNA comprising a guide sequence that binds to a nucleotide sequence that is complementary to SEQ ID NO: 1302 and a second guide RNA comprising a guide sequence that binds to a nucleotide sequence that is complementary to SEQ ID NO: 1305;
j. a first guide RNA comprising a guide sequence that binds to a nucleotide sequence that is complementary to SEQ ID NO: 1302 and a second guide RNA comprising a guide sequence that binds to a nucleotide sequence that is complementary to SEQ ID NO: 1306;
k. a first guide RNA comprising a guide sequence that binds to a nucleotide sequence that is complementary to SEQ ID NO: 1302 and a second guide RNA comprising a guide sequence that binds to a nucleotide sequence that is complementary to SEQ ID NO: 1307;
l. a first guide RNA comprising a guide sequence that binds to a nucleotide sequence that is complementary to SEQ ID NO: 1302 and a second guide RNA comprising a guide sequence that binds to a nucleotide sequence that is complementary to SEQ ID NO: 1308;
m. a first guide RNA comprising a guide sequence that binds to a nucleotide sequence that is complementary to SEQ ID NO: 1303 and a second guide RNA comprising a guide sequence that binds to a nucleotide sequence that is complementary to SEQ ID NO: 1305;
n. a first guide RNA comprising a guide sequence that binds to a nucleotide sequence that is complementary to SEQ ID NO: 1303 and a second guide RNA comprising a guide sequence that binds to a nucleotide sequence that is complementary to SEQ ID NO: 1306;
o. a first guide RNA comprising a guide sequence that binds to a nucleotide sequence that is complementary to SEQ ID NO: 1303 and a second guide RNA comprising a guide sequence that binds to a nucleotide sequence that is complementary to SEQ ID NO: 1307;
p. a first guide RNA comprising a guide sequence that binds to a nucleotide sequence that is complementary to SEQ ID NO: 1303 and a second guide RNA comprising a guide sequence that binds to a nucleotide sequence that is complementary to SEQ ID NO: 1308;
q. a first guide RNA comprising a guide sequence that binds to a nucleotide sequence that is complementary to SEQ ID NO: 1304 and a second guide RNA comprising a guide sequence that binds to a nucleotide sequence that is complementary to SEQ ID NO: 1305;
r. a first guide RNA comprising a guide sequence that binds to a nucleotide sequence that is complementary to SEQ ID NO: 1304 and a second guide RNA comprising a guide sequence that binds to a nucleotide sequence that is complementary to SEQ ID NO: 1306;
s. a first guide RNA comprising a guide sequence that binds to a nucleotide sequence that is complementary to SEQ ID NO: 1304 and a second guide RNA comprising a guide sequence that binds to a nucleotide sequence that is complementary to SEQ ID NO: 1307; or
t. a first guide RNA comprising a guide sequence that binds to a nucleotide sequence that is complementary to SEQ ID NO: 1304 and a second guide RNA comprising a guide sequence that binds to a nucleotide sequence that is complementary to SEQ ID NO: 1308.

In some embodiments, a composition is provided comprising a pair of guide RNAs, or nucleic acids encoding same, targeting an E-box of the DMPK gene, wherein the pair of guide RNAs comprise a first guide RNA comprising a first guide sequence that binds to a nucleotide sequence that is complementary to any one of SEQ ID NOs: 1420, 1428, or 1475, and a second guide RNA comprising a second guide sequence that binds to a nucleotide sequence that is complementary to any one of SEQ ID NOs: 1402, 1411, or 1463. In some embodiments, the pair of guide RNAs comprises:
a. a first guide RNA comprising a guide sequence that binds to a nucleotide sequence that is complementary to SEQ ID NO: 1420 and a second guide RNA comprising a guide sequence that binds to a nucleotide sequence that is complementary to SEQ ID NO: 1402;
b. a first guide RNA comprising a guide sequence that binds to a nucleotide sequence that is complementary to SEQ ID NO: 1420 and a second guide RNA comprising a guide sequence that binds to a nucleotide sequence that is complementary to SEQ ID NO: 1411;
c. a first guide RNA comprising a guide sequence that binds to a nucleotide sequence that is complementary to SEQ ID NO: 1420 and a second RNA comprising a guide guide sequence that binds to a nucleotide sequence that is complementary to SEQ ID NO: 1464;
d. a first guide RNA comprising a guide sequence that binds to a nucleotide sequence that is complementary to SEQ ID NO: 1428 and a second guide RNA comprising a guide sequence that binds to a nucleotide sequence that is complementary to SEQ ID NO: 1402;
e. a first guide RNA comprising a guide sequence that binds to a nucleotide sequence that is complementary to SEQ ID NO: 1428 and a second guide RNA comprising a guide sequence that binds to a nucleotide sequence that is complementary to SEQ ID NO: 1411;
f. a first guide RNA comprising a guide sequence that binds to a nucleotide sequence that is complementary to SEQ ID NO: 1428 and a second guide RNA comprising a guide sequence that binds to a nucleotide sequence that is complementary to SEQ ID NO: 1464;
g. a first guide RNA comprising a guide sequence that binds to a nucleotide sequence that is complementary to SEQ ID NO: 1475 and a second guide RNA comprising a guide sequence that binds to a nucleotide sequence that is complementary to SEQ ID NO: 1402;
h. a first guide RNA comprising a guide sequence that binds to a nucleotide sequence that is complementary to SEQ ID NO: 1475 and a second guide RNA comprising a guide sequence that binds to a nucleotide sequence that is complementary to SEQ ID NO: 1411; or
i. a first guide RNA comprising a guide sequence that binds to a nucleotide sequence that is complementary to SEQ ID NO: 1475 and a second guide RNA comprising a guide sequence that binds to a nucleotide sequence that is complementary to SEQ ID NO: 1464.

In some embodiments, a composition is provided comprising two pairs of guide RNAs (4 total guide RNAs) targeting a TSS and/or an E-box regulatory element of DMPK, wherein the first pair of guide RNAs comprise at least 17, 18, 19, 20, 21, or 22 contiguous nucleotides of a first guide sequence selected from any one of SEQ ID NOs: 1, 2, or 3, and at least 17, 18, 19, 20, 21, or 22 contiguous nucleotides of a second guide sequence selected from any one of SEQ ID NOs: 4, 5, or 6; and the second pair of guide RNAs comprise at least 17, 18, 19, 20, 21, or 22 contiguous nucleotides of a first guide sequence selected from any one of SEQ ID NOs: 7, 8, or 9, and at least 17, 18, 19, 20, 21, or 22 contiguous nucleotides of a second guide sequence selected from any one of SEQ ID NOs: 10, 11, or 12.

In some embodiments, a composition is provided comprising two pairs of guide RNAs (4 total guide RNAs) targeting a TSS and/or an E-box regulatory element of DMPK, wherein in one embodiment, the first pair of guide RNAs comprise a first guide RNA comprising at least 17, 18, 19, 20, 21, or 22 contiguous nucleotides of a guide sequence selected from any one of SEQ ID NOs: 1300, 1301, 1302, 1303, and 1304, and a second guide RNA comprising at least 17, 18, 19, 20, 21, or 22 contiguous nucleotides of a guide sequence selected from any one of SEQ ID NOs: 1305, 1306, 1307, and 1308; and the second pair of guide RNAs comprises a third guide RNA comprising at least 17, 18, 19, 20, 21, or 22 contiguous nucleotides of a guide sequence selected from any one of SEQ ID NOs: 1300, 1301, 1302, 1303, and 1304, and a fourth guide RNA comprising at least 17, 18, 19, 20, 21, or 22 contiguous nucleotides of a guide sequence selected from any one of SEQ ID NOs: 1305, 1306, 1307, and 1308. In some embodiments, the first and second pair of guide RNAs are identical (e.g., the first guide RNA is the same as the third guide RNA and the second guide RNA is the same as the fourth guide RNA). In some embodiments, the first and second pair of guide RNAs are not identical.

In some embodiments, a composition is provided comprising two pairs of guide RNAs (4 total guide RNAs) targeting a TSS and/or an E-box regulatory element of DMPK, wherein in one embodiment, the first pair of guide RNAs comprise a first guide RNA comprising at least 17, 18, 19, 20, 21, or 22 contiguous nucleotides of a guide sequence selected from any one of SEQ ID NOs: 1420, 1428, and 1475, and a second guide RNA comprising at least 17, 18, 19, 20, 21, or 22 contiguous nucleotides of a guide sequence selected from any one of SEQ ID NOs: 1402, 1411, and 1463; and the second pair of guide RNAs comprise a third guide RNA comprising at least 17, 18, 19, 20, 21, or 22 contiguous nucleotides of a first guide sequence selected from any one of SEQ ID NOs: 1420, 1428, and 1475, and a fourth guide RNA comprising at least 17, 18, 19, 20, 21, or 22 contiguous nucleotides of a second guide sequence selected from any one of SEQ ID NOs: 1402, 1411, and 1463. In some embodiments, the first and second pair of guide RNAs are identical (e.g., the first guide RNA is the same as the third guide RNA and the second guide RNA is the same as the fourth guide RNA). In some embodiments, the first and second pair of guide RNAs are not identical. In some embodiments, the guide RNA is associated with a lipid nanoparticle (LNP) or a viral vector to deliver the guide RNAs and compositions to a cell. In some embodiments, the vectors or LNPs associated with the guide RNAs and compositions are for use in preparing a medicament or pharmaceutical formulation for treating a disease or disorder.

In some embodiments, any one or more of the guide RNAs or pairs of guide RNAs disclosed herein is packaged in one or more vectors. Any type of vector, such as any of those described herein, may be used. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector. In some embodiments, the vector comprises a muscle-specific promoter. Exemplary muscle-specific promoters include a muscle creatine kinase promoter, a desmin promoter, an MHCK7 promoter, or an SPc5-12 promoter. See US 2004/0175727 A1; Wang et al., Expert Opin Drug Deliv. (2014) 11, 345-364; Wang et al., Gene Therapy (2008) 15, 1489-1499. In some embodiments, the muscle-specific promoter is a CK8 promoter. In some embodiments, the muscle-specific promoter is a CK8e promoter. In some embodiments, the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter. In some embodiments, the vector further comprises a nucleotide sequence encoding an endonuclease. In some embodiments, the endonuclease is a Cas protein. In some embodiments, the Cas protein is any of the Cas proteins disclosed herein. In some embodiments, the disclosure provides for a composition comprising multiple vectors, wherein at least one of the vectors comprises a nucleotide sequence encoding one or more guide RNAs and at least one of the vectors comprises a nucleotide sequence encoding an endonuclease (e.g., any of the Cas proteins disclosed herein).

In some embodiments, the guide RNA or pairs of guide RNAs are associated with an adeno-associated virus (AAV) vector. The AAV vector can be an AAV1, AAV2, AAV3, AAV4, AAV5, AAV6, AAV7, AAV8, AAVrh10, AAVrh74, or AAV9 vector, where the number following AAV indicates the AAV serotype. Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. In some embodiments, the AAV vector is an AAV serotype 9 vector. In some embodiments, the AAV vector is an AAVrh10 vector. In some embodiments, the AAV vector is an AAVrh74 vector.

In some embodiments, the guide RNA is associated with an LNP or lipoplex. Lipid nanoparticles (LNPs) and lipoplexes are a known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

The compositions described herein are useful for treating a disease or disorder characterized by toxic proteins and transcripts including, but not limited to Myotonic Dystrophy Type 1 (DM1), Myotonic dystrophy type 2 (DM2), Fuchs endothelial corneal dystrophy (FECD), Huntington disease-like 2 (HDL2), Huntington disease (HD), Spinocerebellar ataxia (SCA), Spinal and bulbar muscular atrophy (SBMA), Dentatorubral-pallidoluysian atrophy (DRPLA), Fragile X-associated tremor/ataxia syndrome (FXTAS), Frontotemporal dementia (FTD), amyotrophic lateral sclerosis (ALS), Oculopharyngeal muscular dystrophy (OPMD), Alzheimer's Disease (AD), and Parkinson's Disease (PD). Such compositions may be administered to subjects having or suspected of having any one of the diseases or disorders for the purpose of treating or alleviating the symptoms of those diseases, many of which are known in the art.

In some embodiments, the guide RNA is a single-molecule guide RNA (sgRNA). A sgRNA can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. In some embodiments, as described further in detail below, the sgRNA is modified.

In some embodiments, any of the guides disclosed herein may be used as a research tool, e.g., to study trafficking, expression, and processing by cells.

### A. Modified Guide RNAs

In some embodiments, the guide RNA is chemically modified. A guide RNA comprising one or more modified nucleosides or nucleotides is called a “modified” guide RNA or “chemically modified” guide RNA, to describe the presence of one or more non-naturally and/or naturally occurring components or configurations that are used instead of or in addition to the canonical A, G, C, and U residues. In some embodiments, a modified guide RNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified guide RNAs comprising nucleosides and nucleotides (collectively “residues”) that can have two, three, four, or more modifications. For example, a modified residue can have a modified sugar and a modified nucleobase, or a modified sugar and a modified phosphodiester. In some embodiments, every base of a guide RNA is modified, e.g., all bases have a modified phosphate group, such as a phosphorothioate group. In certain embodiments, all, or substantially all, of the phosphate groups of an guide RNA molecule are replaced with phosphorothioate groups. In some embodiments, modified guide RNAs comprise at least one modified residue at or near the 5′ end of the RNA. In some embodiments, modified guide RNAs comprise at least one modified residue at or near the 3′ end of the RNA.

In some embodiments, the guide RNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified guide RNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the guide RNAs described herein can contain one or more modified nucleosides or nucleotides, e.g., to introduce stability toward intracellular or serum-based nucleases. In some embodiments, the modified guide RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁_₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

In some embodiments, the guide RNA is an sgRNA that is modified, wherein the modification alters one or more 2′ positions and/or phosphodiester linkages.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Modifications of 2′-fluoro (2′-F) are encompassed.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

Abasic nucleotides refer to those which lack nitrogenous bases.

Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide.

In some embodiments, the guide RNA is an sgRNA that is modified, wherein the modification alters one or more 2′ positions and/or phosphodiester linkages. In some embodiments, the modification alters one or more, or all, of the first three nucleotides of the sgRNA and/or of the last three nucleotides of the sgRNA. In some embodiments, the modification includes one or more of a phosphorothioate modification, a 2′-OMe modification, a 2′-O-MOE modification, a 2′-F modification, a 2′-O-methine-4′ bridge modification, a 3′-thiophosphonoacetate modification, or a 2′-deoxy modification.

### B. Ribonucleoprotein Complex

In some embodiments, a composition is encompassed comprising: a) one or more guide RNAs targeting a regulatory element of any one or more of DM1 protein kinase (DMPK), CCHC-type zinc finger nucleic acid binding protein (CNBP), transcription Factor 4 (TCF4), junctophilin 3 (JPH3), huntingtin (HTT), ataxin 1 (ATXN1), ataxin 2 (ATXN2), ataxin 3 (ATXN3), ataxin 7 (ATXN7), ATXN8 opposite strand lncRNA (ATXN8OS), androgen receptor (AR), atrophin 1 (ATN1), fragile X Mental Retardation 1 (FMR1), chromosome 9 open reading frame 72 (C90RF72), polyadenylate-binding protein 2 (PABP2), amyloid precursor protein (APP), presenilin 1 (PSEN1), presenilin 2 (PSEN2), apolipoprotein E4 (APOE4), and synuclein alpha (SNCA); and b) an RNA-targeted endonuclease, including but not limited to any of the Cas proteins known in the art and/or disclosed herein, including, but not limited to, spCas9, saCas9, sluCas9 and variants thereof. In some embodiments, a composition is encompassed comprising: a) one or more guide RNAs comprising one or more guide sequences from Tables 3A-B, 5A-C, and 6A-B and throughout the specification and b) an RNA-targeted endonuclease, including but not limited to any of the Cas proteins known in the art and/or disclosed herein, including, but not limited to, spCas9, saCas9, sluCas9 and variants thereof. In some embodiments, the guide RNA together with a nuclease is called a ribonucleoprotein complex (RNP).

In some embodiments, the disclosure provides for an RNP complex, wherein the guide RNA (e.g., any of the guide RNAs disclosed herein) binds to or is capable of binding to a target sequence in the DMPK gene, or a target sequence bound by any of the sequences disclosed in Tables 1A 3A-B, 5A-C, and 6A-B, wherein the DMPK gene comprises a PAM recognition sequence position upstream of the target sequence, and wherein the RNP cuts at a position that is 3 nucleotides upstream (−3) of the PAM in the DMPK gene. In some embodiments, the RNP also cuts at a position that is 2 nucleotides upstream (−2), 4 nucleotides upstream (−4), 5 nucleotides upstream (−5), or 6 nucleotides upstream (−6) of the PAM in the DMPK gene. In some embodiments, the RNP cuts at a position that is 3 nucleotides upstream (−3) and 4 nucleotides upstream (−4) of the PAM in the DMPK gene.

In some embodiments, the nuclease is a Type II, Type V-A, Type V-B, Type V-C, Type V-U, Type VI-B nuclease. In some embodiments, the nuclease is a Cas9, Cas12i2, Cas12a, Cas12b, Cas12c, Tnp-B like, Cas13a (C2c2), or Cas13b nuclease. In some embodiments, the nuclease is a TAL nuclease, a meganuclease, or a zinc-finger nuclease. In some embodiments, the nuclease is a Cas9 nuclease. In some embodiments, the nuclease is a Cpf1 nuclease.

In some embodiments, the disclosure provides for a Cas nuclease. In some embodiments, the Cas nuclease is a Cpf1 nuclease. In some embodiments, the Cas nuclease is a Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the disclosure provides for a Cas9 nuclease. In some embodiments, the Cas9 nuclease includes those of the type II CRISPR systems of S. pyogenes, S. thermophilus, N. meningitidis, S. aureus, and other prokaryotes, and modified (e.g., engineered or mutant) versions thereof. See, e.g., US2016/0312198 A1; US 2016/0312199 A1. In some embodiments, the Cas nuclease is the Cas9 nuclease from Streptococcus pyogenes (SpCas9). In some embodiments, the Cas nuclease is the Cas9 nuclease from Staphylococcus lugdunensis (SluCas9). In some embodiments, the Cas9 nuclease is from Staphylococcus aureus (SaCas9). In some embodiments, the Cas protein is not a Cas nickase or Cas9 nickase (D10A mutant of Cas9). In some embodiments, the Cas protein is not a Cas nickase and CRISPR interference is not used. In particular embodiments, the Cas protein is not a nuclease-deficient/nuclease-dead Cas protein.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In some embodiments, the Cas protein (e.g., Cas9) is modified to contain only one functional nuclease domain. For example, the agent protein may be modified such that one of the nuclease domains is mutated or fully or partially deleted to reduce its nucleic acid cleavage activity.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell Oct. 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1_FRATN)). Further exemplary amino acid substitutions include D10A and N580A (based on the S. aureus Cas9 protein). See, e.g., Friedland et al., 2015, Genome Biol., 16:257.

In some embodiments, the Cas protein (e.g., Cas9) lacks cleavase activity. In some embodiments, the Cas protein comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the Cas9 lacking cleavase activity or the dCas DNA-binding polypeptide is a version of a Cas nuclease (e.g., a Cas9 nuclease discussed above) in which its endonucleolytic active sites are inactivated, e.g., by one or more alterations (e.g., point mutations) in its catalytic domains. See, e.g., US 2014/0186958 A1; US 2015/0166980 A1. In some embodiments, the Cas protein is not a nuclease dead caspase or nickase (e.g., not a dCas protein or nickase). In some embodiments, the disclosure specifically excludes the use of a catalytically-inactive Cas protein.

In some embodiments, the RNA-targeted endonuclease or Cas protein (e.g., Cas9) comprises one or more heterologous functional domains (e.g., is or comprises a fusion polypeptide).

In some embodiments, the heterologous functional domain may facilitate transport of the Cas protein (e.g., Cas9) into the nucleus of a cell. For example, the heterologous functional domain may be a nuclear localization signal (NLS). In some embodiments, the Cas protein may be fused with 1-10 NLS(s). In some embodiments, the Cas protein may be fused with 1-5 NLS(s). In some embodiments, the Cas protein may be fused with one NLS. Where one NLS is used, the NLS may be attached at the N-terminus or the C-terminus of the Cas protein sequence, and may be directly attached or attached via a linker. In some embodiments, the linker is between 3-15, 3-12, 3-10, 3-8, 3-5 amino acids in length. In some embodiments, the linker comprises glycine. In some embodiments, the linker comprises serine. It may also be inserted within the Cas protein sequence. In other embodiments, the Cas protein may be fused with more than one NLS. In some embodiments, the Cas protein may be fused with 2, 3, 4, or 5 NLSs. In some embodiments, the Cas protein may be fused with two NLSs. In certain circumstances, the two NLSs may be the same (e.g., two SV40 NLSs) or different. In some embodiments, the Cas protein is fused with one or more SV40 NLSs. In some embodiments, the SV40 NLS comprises the amino acid sequence of SEQ ID NO: 24 (PKKKRKV). In some embodiments, the Cas protein (e.g., the SluCas9 protein) is fused to one or more nucleoplasmin NLSs. In some embodiments, the Cas protein is fused to one or more c-myc NLSs. In some embodiments, the Cas protein is fused to one or more E1A NLSs. In some embodiments, the Cas protein is fused to one or more BP (bipartite) NLSs. In some embodiments, the nucleoplasmin NLS comprises the amino acid sequence of SEQ ID NO: 25 (KRPAATKKAGQAKKKK). In some embodiments, the Cas protein is fused with a c-Myc NLS. In some embodiments, the c-Myc NLS is SEQ ID NO: 1506 (PAAKKKKLD) and/or is encoded by the nucleic acid sequence of SEQ ID NO: 1507 (CCGGCAGCTAAGAAAAAGAAACTGGAT). In some embodiments, the is fused to two SV40 NLS sequences linked at the carboxy terminus. In some embodiments, the Cas protein may be fused with two NLSs, one linked at the N-terminus and one at the C-terminus. In some embodiments, the Cas protein may be fused with 3 NLSs. In some embodiments, the Cas protein may be fused with no NLS. In some embodiments, the Cas protein is fused to an SV40 NLS and to a nucleoplasmin NLS. In some embodiments, the SV40 NLS is fused to the C-terminus of the Cas protein, while the nucleoplasmin NLS is fused to the N-terminus of the Cas protein. In some embodiments, the SV40 NLS is fused to the N-terminus of the Cas protein, while the nucleoplasmin NLS is fused to the C-terminus of the Cas protein. In some embodiments, the SV40 NLS is fused to the Cas protein by means of a linker. In some embodiments, the nucleoplasmin NLS is fused to the Cas protein by means of a linker. In some embodiments, the Cas may be fused with an NLS on the C-terminus and does not comprise an NLS fused on the N-terminus. In some embodiments, the Cas may be fused with an NLS on the N-terminus and does not comprise an NLS fused on the C-terminus. In some embodiments, the Cas protein is fused to an SV40 NLS and to a nucleoplasmin NLS. In some embodiments, the SV40 NLS is fused to the C-terminus of the Cas, while the nucleoplasmin NLS is fused to the N-terminus of the Cas protein. In some embodiments, the SV40 NLS is fused to the N-terminus of the Cas, while the nucleoplasmin NLS is fused to the C-terminus of the Cas protein. In some embodiments, a c-myc NLS is fused to the N-terminus of the Cas and an SV40 NLS and/or nucleoplasmin NLS is fused to the C-terminus of the Cas. In some embodiments, a c-myc NLS is fused to the N-terminus of the Cas (e.g., by means of a linker such as GSVD (SEQ ID NO: 1508)), an SV40 NLS is fused to the C-terminus of the Cas (e.g., by means of a linker such as GSGS (SEQ ID NO: 1509)), and a nucleoplasmin NLS is fused to the C-terminus of the SV-40 NLS (e.g., by means of a linker such as GSGS (SEQ ID NO: 1510)). In some embodiments, the SV40 NLS is fused to the Cas protein by means of a linker. In some embodiments, the nucleoplasmin NLS is fused to the Cas protein by means of a linker.

In some embodiments, an additional domain may be: a) fused to the N- or C-terminus of the Cas protein (e.g., a Cas9 protein), b) fused to the N-terminus of an NLS fused to the N-terminus of a Cas protein, or c) fused to the C-terminus of an NLS fused to the C-terminus of a Cas protein. In some embodiments, the additional domain is a destabilizing domain. In some embodiments, a Cas protein (e.g., a Cas9 protein) fused to a destabilizing domain is destabilized and degraded by a cell expressing the Cas protein fused to the destabilizing domain. In some embodiments, a Cas protein (e.g., a Cas9 protein) fused to a destabilizing domain is destabilized and degraded by a cell expressing the Cas protein fused to the destabilizing domain in the absence of a small molecule that specifically binds to the destabilizing domain. In some embodiments, a Cas protein (e.g., a Cas9 protein) fused to a destabilizing domain is stabilized and functional in the presence of a small molecule that specifically binds to the destabilizing domain.

In some embodiments, the heterologous functional domain may be capable of modifying the intracellular half-life of the Cas protein (e.g., Cas9). In some embodiments, the half-life of the Cas protein may be increased. In some embodiments, the half-life of the Cas protein may be reduced. In some embodiments, the heterologous functional domain may be capable of increasing the stability of the Cas protein. In some embodiments, the heterologous functional domain may be capable of reducing the stability of the Cas protein. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the Cas protein may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyani, Midoriishi-Cyan), red fluorescent proteins (e.g., mKate, mKate2, mPlum, DsRed monomer, mCherry, mRFPI, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AUi, AUS, E, ECS, E2, FLAG, HA, nus, Softag 1, Softag 3, Strep, SBP, Glu-Glu, HSV, KT3, S, Si, T7, V5, VSV-G, 6×His, 8×His, biotin carboxyl carrierprotein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the Cas protein to a specific organelle, cell type, tissue, or organ. In some embodiments, the heterologous functional domain may target the Cas protein to muscle.

In further embodiments, the heterologous functional domain may be an effector domain. When the Cas protein is directed to its target sequence, e.g., when a Cas9 is directed to a target sequence by a guide RNA, the effector domain may modify or affect the target sequence. In some embodiments, the effector domain may be chosen from a nucleic acid binding domain or a nuclease domain (e.g., a non-Cas nuclease domain). In some embodiments, the heterologous functional domain is a nuclease, such as a FokI nuclease. See, e.g., U.S. Pat. No. 9,023,649.

As used herein, “Streptococcus pyogenes Cas9” may also be referred to as SpCas9, and includes wild type SpCas9 (e.g., SEQ ID NO: 710) and variants thereof. A variant of SpCas9 comprises one or more amino acid changes as compared to SEQ ID NO: 710, including insertion, deletion, or substitution of one or more amino acids, or a chemical modification to one or more amino acids. In some embodiments, the variant includes mutations at D10A or H840A (which creates a single-strand nickase), or mutations at D10A and H840A (which abrogates nuclease activity; this mutant is known as dead Cas9 or dCas9).

As used herein, “Staphylococcus aureus Cas9” may also be referred to as SaCas9, and includes wild type SaCas9 (e.g., SEQ ID NO: 711) and variants thereof. A variant of SaCas9 comprises one or more amino acid changes as compared to SEQ ID NO: 711, including insertion, deletion, or substitution of one or more amino acids, or a chemical modification to one or more amino acids.

As used herein, “Staphylococcus lugdunensis Cas9” may also be referred to as SluCas9, and includes wild type SluCas9 (e.g., SEQ ID NO: 712) and variants thereof. A variant of SluCas9 comprises one or more amino acid changes as compared to SEQ ID NO: 712, including insertion, deletion, or substitution of one or more amino acids, or a chemical modification to one or more amino acids.

In some embodiments, the nucleic acid encoding SaCas9 encodes an SaCas9 comprising an amino acid sequence that is at least 80%, 85%, 90%, 91%, 92%, 93%, 94%, 95%, 96%, 97%, 98%, 99% or 100% identical to the sequence of SEQ ID NO: 711:

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYNNDLIKINGELYRVIGVNNDLLNRIEVNMIDITYREYLENMN
DKRPPRIIKTIASKTQSIKKYSTDILGNLYEVKSKKHPQIIKKG.

In some embodiments, the nucleic acid encoding SaCas9 comprises the nucleic acid of SEQ ID NO: 914:

AAGCGCAATTACATCCTGGGCCTGGATATCGGCATCACCTCCGTGGGCTACG
GCATCATCGACTATGAGACACGGGATGTGATCGACGCCGGCGTGAGACTGTTCAAGGAG
GCCAACGTGGAGAACAATGAGGGCCGGCGGAGCAAGAGGGGAGCAAGGCGCCTGAAGC
GGAGAAGGCGCCACAGAATCCAGAGAGTGAAGAAGCTGCTGTTCGATTACAACCTGCTG
ACCGACCACTCCGAGCTGTCTGGCATCAATCCTTATGAGGCCCGGGTGAAGGGCCTGTCC
CAGAAGCTGTCTGAGGAGGAGTTTTCTGCCGCCCTGCTGCACCTGGCAAAGAGGAGAGG
CGTGCACAACGTGAATGAGGTGGAGGAGGACACCGGCAACGAGCTGAGCACAAAGGAG
CAGATCAGCCGCAATTCCAAGGCCCTGGAGGAGAAGTATGTGGCCGAGCTGCAGCTGGA
GCGGCTGAAGAAGGATGGCGAGGTGAGGGGCTCCATCAATCGCTTCAAGACCTCTGACT
ACGTGAAGGAGGCCAAGCAGCTGCTGAAGGTGCAGAAGGCCTACCACCAGCTGGATCAG
AGCTTTATCGATACATATATCGACCTGCTGGAGACCAGGCGCACATACTATGAGGGACC
AGGAGAGGGCTCCCCCTTCGGCTGGAAGGACATCAAGGAGTGGTACGAGATGCTGATGG
GCCACTGCACCTATTTTCCAGAGGAGCTGAGATCCGTGAAGTACGCCTATAACGCCGATC
TGTACAACGCCCTGAATGACCTGAACAACCTGGTCATCACCAGGGATGAGAACGAGAAG
CTGGAGTACTATGAGAAGTTCCAGATCATCGAGAACGTGTTCAAGCAGAAGAAGAAGCC
TACACTGAAGCAGATCGCCAAGGAGATCCTGGTGAACGAGGAGGACATCAAGGGCTACC
GCGTGACCAGCACAGGCAAGCCAGAGTTCACCAATCTGAAGGTGTATCACGATATCAAG
GACATCACAGCCCGGAAGGAGATCATCGAGAACGCCGAGCTGCTGGATCAGATCGCCAA
GATCCTGACCATCTATCAGAGCTCCGAGGACATCCAGGAGGAGCTGACCAACCTGAATA
GCGAGCTGACACAGGAGGAGATCGAGCAGATCAGCAATCTGAAGGGCTACACCGGCAC
ACACAACCTGTCCCTGAAGGCCATCAATCTGATCCTGGATGAGCTGTGGCACACAAACG
ACAATCAGATCGCCATCTTTAACAGGCTGAAGCTGGTGCCAAAGAAGGTGGACCTGAGC
CAGCAGAAGGAGATCCCAACCACACTGGTGGACGATTTCATCCTGTCCCCCGTGGTGAA
GCGGAGCTTCATCCAGAGCATCAAAGTGATCAACGCCATCATCAAGAAGTACGGCCTGC
CCAATGATATCATCATCGAGCTGGCCAGGGAGAAGAACTCTAAGGACGCCCAGAAGATG
ATCAATGAGATGCAGAAGAGGAACCGCCAGACCAATGAGCGGATCGAGGAGATCATCA
GAACCACAGGCAAGGAGAACGCCAAGTACCTGATCGAGAAGATCAAGCTGCACGATAT
GCAGGAGGGCAAGTGTCTGTATAGCCTGGAGGCCATCCCTCTGGAGGACCTGCTGAACA
ATCCATTCAACTACGAGGTGGATCACATCATCCCCCGGAGCGTGAGCTTCGACAATTCCT
TTAACAATAAGGTGCTGGTGAAGCAGGAGGAGAACTCTAAGAAGGGCAATAGGACCCCT
TTCCAGTACCTGTCTAGCTCCGATTCTAAGATCAGCTACGAGACCTTCAAGAAGCACATC
CTGAATCTGGCCAAGGGCAAGGGCCGCATCTCTAAGACCAAGAAGGAGTACCTGCTGGA
GGAGCGGGACATCAACAGATTCAGCGTGCAGAAGGACTTCATCAACCGGAATCTGGTGG
ACACCAGATACGCCACACGCGGCCTGATGAATCTGCTGCGGTCCTATTTCAGAGTGAACA
ATCTGGATGTGAAGGTGAAGAGCATCAACGGCGGCTTCACCTCCTTTCTGCGGAGAAAG
TGGAAGTTTAAGAAGGAGAGAAACAAGGGCTATAAGCACCACGCCGAGGATGCCCTGAT
CATCGCCAATGCCGACTTCATCTTTAAGGAGTGGAAGAAGCTGGACAAGGCCAAGAAAG
TGATGGAGAACCAGATGTTCGAGGAGAAGCAGGCCGAGAGCATGCCCGAGATCGAGAC
CGAGCAGGAGTACAAGGAGATTTTCATCACACCTCACCAGATCAAGCACATCAAGGACT
TCAAGGACTACAAGTATTCCCACAGGGTGGATAAGAAGCCCAACCGCGAGCTGATCAAT
GACACCCTGTATTCTACAAGGAAGGACGATAAGGGCAATACCCTGATCGTGAACAATCT
GAACGGCCTGTACGACAAGGATAATGACAAGCTGAAGAAGCTGATCAACAAGAGCCCC
GAGAAGCTGCTGATGTACCACCACGATCCTCAGACATATCAGAAGCTGAAGCTGATCAT
GGAGCAGTACGGCGACGAGAAGAACCCACTGTATAAGTACTATGAGGAGACCGGCAACT
ACCTGACAAAGTATTCCAAGAAGGATAATGGCCCCGTGATCAAGAAGATCAAGTACTAT
GGCAACAAGCTGAATGCCCACCTGGACATCACCGACGATTACCCCAACAGCCGGAATAA
GGTGGTGAAGCTGAGCCTGAAGCCATACAGGTTCGACGTGTACCTGGACAACGGCGTGT
ATAAGTTTGTGACAGTGAAGAATCTGGATGTGATCAAGAAGGAGAACTACTATGAAGTG
AATAGCAAGTGCTACGAGGAGGCCAAGAAGCTGAAGAAGATCAGCAACCAGGCCGAGT
TCATCGCCTCTTTTTACAACAATGACCTGATCAAGATCAATGGCGAGCTGTATAGAGTGA
TCGGCGTGAACAATGATCTGCTGAACCGCATCGAAGTGAATATGATCGACATCACCTACC
GGGAGTATCTGGAGAACATGAATGATAAGAGGCCCCCTCGCATCATCAAGACCATCGCC
TCTAAGACACAGAGCATCAAGAAGTACTCTACAGACATCCTGGGCAACCTGTATGAGGT
GAAGAGCAAGAAGCACCCTCAGATCATCAAGAAGGGC.

In some embodiments comprising a nucleic acid encoding SaCas9, the SaCas9 comprises an amino acid sequence of SEQ ID NO: 711.

In some embodiments, the SaCas9 is a variant of the amino acid sequence of SEQ ID NO: 711. In some embodiments, the SaCas9 comprises an amino acid other than an E at the position corresponding to position 781 of SEQ ID NO: 711. In some embodiments, the SaCas9 comprises an amino acid other than an N at the position corresponding to position 967 of SEQ ID NO: 711. In some embodiments, the SaCas9 comprises an amino acid other than an R at the position corresponding to position 1014 of SEQ ID NO: 711. In some embodiments, the SaCas9 comprises a K at the position corresponding to position 781 of SEQ ID NO: 711. In some embodiments, the SaCas9 comprises a K at the position corresponding to position 967 of SEQ ID NO: 711. In some embodiments, the SaCas9 comprises an H at the position corresponding to position 1014 of SEQ ID NO: 711. In some embodiments, the SaCas9 comprises an amino acid other than an E at the position corresponding to position 781 of SEQ ID NO: 711; an amino acid other than an N at the position corresponding to position 967 of SEQ ID NO: 711; and an amino acid other than an R at the position corresponding to position 1014 of SEQ ID NO: 711. In some embodiments, the SaCas9 comprises a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid other than an R at the position corresponding to position 244 of SEQ ID NO: 711. In some embodiments, the SaCas9 comprises an amino acid other than an N at the position corresponding to position 412 of SEQ ID NO: 711. In some embodiments, the SaCas9 comprises an amino acid other than an N at the position corresponding to position 418 of SEQ ID NO: 711. In some embodiments, the SaCas9 comprises an amino acid other than an R at the position corresponding to position 653 of SEQ ID NO: 711. In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; and an amino acid other than an R at the position corresponding to position 653 of SEQ ID NO: 711. In some embodiments, the SaCas9 comprises an A at the position corresponding to position 244 of SEQ ID NO: 711. In some embodiments, the SaCas9 comprises an A at the position corresponding to position 412 of SEQ ID NO: 711. In some embodiments, the SaCas9 comprises an A at the position corresponding to position 418 of SEQ ID NO: 711. In some embodiments, the SaCas9 comprises an A at the position corresponding to position 653 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; and an A at the position corresponding to position 653 of SEQ ID NO: 711.

In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; an amino acid other than an R at the position corresponding to position 653 of SEQ ID NO: 711; an amino acid other than an E at the position corresponding to position 781 of SEQ ID NO: 711; an amino acid other than an N at the position corresponding to position 967 of SEQ ID NO: 711; and an amino acid other than an R at the position corresponding to position 1014 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; an A at the position corresponding to position 653 of SEQ ID NO: 711; a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 715 (designated herein as SaCas9-KKH or SACAS9KKH):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYKNDLIKINGELYRVIGVNNDLLNRIEVNMIDITYREYLENMN
DKRPPHIIKTIASKTQSIKKYSTDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 716 (designated herein as SaCas9-HF):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELASVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDAQIAIFARLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATAGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYNNDLIKINGELYRVIGVNNDLLNRIEVNMIDITYREYLENMN
DKRPPRIIKTIASKTQSIKKYSTDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 717 (designated herein as SaCas9-KKH-HF):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELASVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDAQIAIFARLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATAGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYKNDLIKINGELYRVIGVNNDLLNRIEVNMIDITYREYLENMN
DKRPPHIIKTIASKTQSIKKYSTDILGNLYEVKSKKHPQIIKKG.

In some embodiments, the nucleic acid encoding SluCas9 encodes a SluCas9 comprising an amino acid sequence that is at least 80%, 85%, 90%, 91%, 92%, 93%, 94%, 95%, 96%, 97%, 98%, 99% or 100% identical to the sequence of SEQ ID NO: 712:

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELRSVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRNQMEIFTHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATRELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRQLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
RIKKTIGKKVNSIEKLTTDVLGNVFTNTQYTKPQLLFKRGN.

In some embodiments, the SluCas9 is a variant of the amino acid sequence of SEQ ID NO: 712. In some embodiments, the SluCas9 comprises an amino acid other than an Q at the position corresponding to position 781 of SEQ ID NO: 712. In some embodiments, the SluCas9 comprises an amino acid other than an R at the position corresponding to position 1013 of SEQ ID NO: 712. In some embodiments, the SluCas9 comprises a K at the position corresponding to position 781 of SEQ ID NO: 712. In some embodiments, the SluCas9 comprises a K at the position corresponding to position 966 of SEQ ID NO: 712. In some embodiments, the SluCas9 comprises an H at the position corresponding to position 1013 of SEQ ID NO: 712. In some embodiments, the SluCas9 comprises an amino acid other than an Q at the position corresponding to position 781 of SEQ ID NO: 712; and an amino acid other than an R at the position corresponding to position 1013 of SEQ ID NO: 712. In some embodiments, the SluCas9 comprises a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid other than an R at the position corresponding to position 246 of SEQ ID NO: 712. In some embodiments, the SluCas9 comprises an amino acid other than an N at the position corresponding to position 414 of SEQ ID NO: 712. In some embodiments, the SluCas9 comprises an amino acid other than a T at the position corresponding to position 420 of SEQ ID NO: 712. In some embodiments, the SluCas9 comprises an amino acid other than an R at the position corresponding to position 655 of SEQ ID NO: 712. In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; and an amino acid other than an R at the position corresponding to position 655 of SEQ ID NO: 712. In some embodiments, the SluCas9 comprises an A at the position corresponding to position 246 of SEQ ID NO: 712. In some embodiments, the SluCas9 comprises an A at the position corresponding to position 414 of SEQ ID NO: 712. In some embodiments, the SluCas9 comprises an A at the position corresponding to position 420 of SEQ ID NO: 712. In some embodiments, the SluCas9 comprises an A at the position corresponding to position 655 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; and an A at the position corresponding to position 655 of SEQ ID NO: 712.

In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; an amino acid other than an R at the position corresponding to position 655 of SEQ ID NO: 712; an amino acid other than an Q at the position corresponding to position 781 of SEQ ID NO: 712; a K at the position corresponding to position 966 of SEQ ID NO: 712; and an amino acid other than an R at the position corresponding to position 1013 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; an A at the position corresponding to position 655 of SEQ ID NO: 712; a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 718 (designated herein as SluCas9-KH or SLUCAS9KH):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELRSVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRNQMEIFTHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATRELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRKLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
HIKKTIGKKVNSIEKLTTDVLGNVFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 719 (designated herein as SluCas9-HF):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELASVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRAQMEIFAHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATAELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRQLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
RIKKTIGKKVNSIEKLTTDVLGNVFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 720 (designated herein as SluCas9-HF-KH):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELASVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRAQMEIFAHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATAELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRKLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
HIKKTIGKKVNSIEKLTTDVLGNVFTNTQYTKPQLLFKRGN.

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 721 (designated herein as sRGN1):

MNQKFILGLDIGITSVGYGLIDYETKNIIDAGVRLFPEANVENNEGRRSKRGSRRLKRRRIHRL
DRVKHLLAEYDLLDLTNIPKSTNPYQTRVKGLNEKLSKDELVIALLHIAKRRGIHNVDVAAD
KEETASDSLSTKDQINKNAKFLESRYVCELQKERLENEGHVRGVENRFLTKDIVREAKKIIDT
QMQYYPEIDETFKEKYISLVETRREYFEGPGKGSPFGWEGNIKKWFEQMMGHCTYFPEELRS
VKYSYSAELFNALNDLNNLVITRDEDAKLNYGEKFQIIENVFKQKKTPNLKQIAIEIGVHETEI
KGYRVNKSGTPEFTEFKLYHDLKSIVFDKSILENEAILDQIAEILTIYQDEQSIKEELNKLPEILN
EQDKAEIAKLIGYNGTHRLSLKCIHLINEELWQTSRNQMEIFNYLNIKPNKVDLSEQNKIPKD
MVNDFILSPVVKRTFIQSINVINKVIEKYGIPEDIIIELARENNSDDRKKFINNLQKKNEATRKRI
NEIIGQTGNQNAKRIVEKIRLHDQQEGKCLYSLKDIPLEDLLRNPNNYDIDHIIPRSVSFDDSM
HNKVLVRREQNAKKNNQTPYQYLTSGYADIKYSVFKQHVLNLAENKDRMTKKKREYLLEE
RDINKFEVQKEFINRNLVDTRYATRELTNYLKAYFSANNMNVKVKTINGSFTDYLRKVWKF
KKERNHGYKHHAEDALIIANADFLFKENKKLKAVNSVLEKPEIETKQLDIQVDSEDNYSEMFI
IPKQVQDIKDFRNFKYSHRVDKKPNRQLINDTLYSTRKKDNSTYIVQTIKDIYAKDNTTLKKQ
FDKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 722 (designated herein as sRGN2):

MNQKFILGLDIGITSVGYGLIDYETKNIIDAGVRLFPEANVENNEGRRSKRGSRRLKRRRIHRL
ERVKSLLSEYKIISGLAPTNNQPYNIRVKGLTEQLTKDELAVALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGQGSPFGWNGDLKKWYEMLMGHCTYFPQELRSVKYA
YSADLFNALNDLNNLIIQRDNSEKLEYHEKYHIIENVFKQKKKPTLKQIAKEIGVNPEDIKGYR
ITKSGTPEFTEFKLYHDLKSVLFDQSILENEDVLDQIAEILTIYQDKDSIKSKLTELDILLNEEDK
ENIAQLTGYNGTHRLSLKCIRLVLEEQWYSSRNQMEIFTHLNIKPKKINLTAANKIPKAMIDEF
ILSPVVKRTFIQSINVINKVIEKYGIPEDIIIELARENNSDDRKKFINNLQKKNEATRKRINEIIGQ
TGNQNAKRIVEKIRLHDQQEGKCLYSLESIALMDLLNNPQNYEVDHIIPRSVAFDNSIHNKVL
VKQIENSKKGNRTPYQYLNSSDAKLSYNQFKQHILNLSKSKDRISKKKKDYLLEERDINKFEV
QKEFINRNLVDTRYATRELTSYLKAYFSANNMDVKVKTINGSFTNHLRKVWRFDKYRNHGY
KHHAEDALIIANADFLFKENKKLKAVNSVLEKPEIETKQLDIQVDSEDNYSEMFIIPKQVQDIK
DFRNFKYSHRVDKKPNRQLINDTLYSTRKKDNSTYIVQTIKDIYAKDNTTLKKQFDKSPEKFL
MYQHDPRTFEKLEVIMKQYANEKNPLAKYHEETGEYLTKYSKKNNGPIVKSLKYIGNKLGS
HLDVTHQFKSSTKKLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYKEYCELNNIKGEPRIK
KTIGKKVNSIEKLTTDVLGNVFTNTQYTKPQLLFKRGN

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 723 (designated herein as sRGN3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 724 (designated herein as sRGN3.1):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKNYRFDVYLTEKGYKFVTIAYLNVFKKDNYYYIP
KDKYQELKEKKKIKDTDQFIASFYKNDLIKLNGDLYKIIGVNSDDRNIIELDYYDIKYKDYCEI
NNIKGEPRIKKTIGKKTESIEKFTTDVLGNLYLHSTEKAPQLIFKRGL

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 725 (designated herein as sRGN3.2):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKFSHRVDKKPNRQLINDTLYSTRMKDEHDYIVQTITDIYGKDNTNLKKQ
FNKNPEKFLMYQNDPKTFEKLSIIMKQYSDEKNPLAKYYEETGEYLTKYSKKNNGPIVKKIK
LLGNKVGNHLDVTNKYENSTKKLVKLSIKNYRFDVYLTEKGYKFVTIAYLNVFKKDNYYYI
PKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 726 (designated herein as sRGN3.3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTSYLKAYFSANNMDVKVKTINGSFTNHLRKVWRFD
KYRNHGYKHHAEDALIIANADFLFKENKKLQNTNKILEKPTIENNTKKVTVEKEEDYNNVFE
TPKLVEDIKQYRDYKFSHRVDKKPNRQLINDTLYSTRMKDEHDYIVQTITDIYGKDNTNLKK
QFNKNPEKFLMYQNDPKTFEKLSIIMKQYSDEKNPLAKYYEETGEYLTKYSKKNNGPIVKKI
KLLGNKVGNHLDVTNKYENSTKKLVKLSIKNYRFDVYLTEKGYKFVTIAYLNVFKKDNYYY
IPKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 727 (designated herein as sRGN4):

MNQKFILGLDIGITSVGYGLIDYETKNIIDAGVRLFPEANVENNEGRRSKRGSRRLKRRRIHRL
ERVKKLLEDYNLLDQSQIPQSTNPYAIRVKGLSEALSKDELVIALLHIAKRRGIHNINVSSEDE
DASNELSTKEQINRNNKLLKDKYVCEVQLQRLKEGQIRGEKNRFKTTDILKEIDQLLKVQKD
YHNLDIDFINQYKEIVETRREYFEGPGKGSPYGWEGDPKAWYETLMGHCTYFPDELRSVKY
AYSADLFNALNDLNNLVIQRDGLSKLEYHEKYHIIENVFKQKKKPTLKQIANEINVNPEDIKG
YRITKSGKPEFTSFKLFHDLKKVVKDHAILDDIDLLNQIAEILTIYQDKDSIVAELGQLEYLMS
EADKQSISELTGYTGTHSLSLKCMNMIIDELWHSSMNQMEVFTYLNMRPKKYELKGYQRIPT
DMIDDAILSPVVKRTFIQSINVINKVIEKYGIPEDIIIELARENNSDDRKKFINNLQKKNEATRKR
INEIIGQTGNQNAKRIVEKIRLHDQQEGKCLYSLESIPLEDLLNNPNHYEVDHIIPRSVSFDNSY
HNKVLVKQSENSKKSNLTPYQYFNSGKSKLSYNQFKQHILNLSKSQDRISKKKKEYLLEERDI
NKFEVQKEFINRNLVDTRYATRELTNYLKAYFSANNMNVKVKTINGSFTDYLRKVWKFKKE
RNHGYKHHAEDALIIANADFLFKENKKLKAVNSVLEKPEIETKQLDIQVDSEDNYSEMFIIPK
QVQDIKDFRNFKYSHRVDKKPNRQLINDTLYSTRKKDNSTYIVQTIKDIYAKDNTTLKKQFD
KSPEKFLMYQHDPRTFEKLEVIMKQYANEKNPLAKYHEETGEYLTKYSKKNNGPIVKSLKYI
GNKLGSHLDVTHQFKSSTKKLVKLSIKPYRFDVYLTDKGYKFITISYLDVLKKDNYYYIPEQK
YDKLKLGKAIDKNAKFIASFYKNDLIKLDGEIYKIIGVNSDTRNMIELDLPDIRYKEYCELNNI
KGEPRIKKTIGKKVNSIEKLTTDVLGNVFTNTQYTKPQLLFKRGN

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 728 (designated herein as Staphylococcus hyicus Cas9 or ShyCas9):

MNNYILGLDIGITSVGYGIVDSDTREIKDAGVRLFPEANVDNNEGRRSKRGARRLKRRRIHRL
DRVKHLLAEYDLLDLTNIPKSTNPYQTRVKGLNEKLSKDELVIALLHIAKRRGIHNVNVMMD
DNDSGNELSTKDQLKKNAKALSDKYVCELQLERFEQDYKVRGEKNRFKTEDFVREARKLLE
TQSKFFEIDQTFIMRYIELIETRREYFEGPGKGSPFGWEGNIKKWFEQMMGHCTYFPEELRSV
KYSYSAELFNALNDLNNLVITRDEDAKLNYGEKFQIIENVFKQKKTPNLKQIAIEIGVHETEIK
GYRVNKSGKPEFTQFKLYHDLKNIFKDPKYLNDIQLMDNIAEIITIYQDAESIIKELNQLPELLS
EREKEKISALSGYSGTHRLSLKCINLLLDDLWESSLNQMELFTKLNLKPKKIDLSQQHKIPSKL
VDDFILSPVVKRAFIQSIQVVNAIIDKYGLPEDIIIELARENNSDDRRKFLNQLQKQNEETRKQV
EKVLREYGNDNAKRIVQKIKLHNMQEGKCLYSLKDIPLEDLLRNPHHYEVDHIIPRSVAFDNS
MHNKVLVRADENSKKGNRTPYQYLNSSESSLSYNEFKQHILNLSKTKDRITKKKREYLLEER
DINKFDVQKEFINRNLVDTRYATRELTSLLKAYFSANNLDVKVKTINGSFTNYLRKVWKFDK
DRNKGYKHHAEDALIIANADFLFKHNKKLRNINKVLDAPSKEVDKKRVTVQSEDEYNQIFED
TQKAQAIKKFEIRKFSHRVDKKPNRQLINDTLYSTRNIDGIEYVVESIKDIYSVNNDKVKTKFK
KDPHRLLMYRNDPQTFEKFEKVFKQYESEKNPFAKYYEETGEKIRKFSKTGQGPYINKIKYLR
ERLGRHCDVTNKYINSRNKIVQLKIYSYRFDIYQYGNNYKMITISYIDLEQKSNYYYISREKYE
QKKKDKQIDDSYKFIGSFYKNDIINYNGEMYRVIGVNDSEKNKIQLDMIDISIKDYMELNNIK
KTGVIYKTIGKSTTHIEKYTTDILGNLYKAAPPKKPQLIFK

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 729 (designated herein as Staphylococcus microti Cas9 or Smi Cas9):

MEKDYILGLDIGIGSVGYGLIDYDTKSIIDAGVRLFPEANADNNLGRRAKRGARRLKRRRIHR
LERVKSLLSEYKIISGLAPTNNQPYNIRVKGLTEQLTKDELAVALLHIAKRRGIHNVDVAADK
EETASDSLSTKDQINKNAKFLESRYVCELQKERLENEGHVRGVENRFLTKDIVREAKKIIDTQ
MQYYPEIDETFKEKYISLVETRREYYEGPGKGSPYGWDADVKKWYQLMMGHCTYFPVEFRS
VKYAYTADLYNALNDLNNLTIARDDNPKLEYHEKYHIIENVFKQKRNPTLKQIAKEIGVNDI
NISGYRVTKSGKPQFTSFKLFHDLKKVVKDHAILDDIDLLNQIAEILTIYQDKDSIVAELGQLE
YLMSEADKQSISELTGYTGTHSLSLKCMNMIIDELWHSSMNQMEVFTYLNMRPKKYELKGY
QRIPTDMIDDAILSPVVKRSFKQAIGVVNAIIKKYGLPKDIIIELARESNSAEKSRYLRAIQKKN
EKTRERIEAIIKEYGNENAKGLVQKIKLHDAQEGKCLYSLKDIPLEDLLRNPNNYDIDHIIPRS
VSFDDSMHNKVLVRREQNAKKNNQTPYQYLTSGYADIKYSVFKQHVLNLAENKDRMTKKK
REYLLEERNINKYDVQKEFINRNLVDTRYTTRELTTLLKTYFTINNLDVKVKTINGSFTDFLR
KRWGFKKNRDEGYKHHAEDALIIANADYLFKEHKLLKEIKDVSDLAGDERNSNVKDEDQYE
EVFGGYFKIEDIKKYKIKKFSHRVDKKPNRQLINDTIYSTRVKDDKRYLINTLKNLYDKSNGD
LKERMQKDPESLLMYHHDPQTFEKLKIVMSQYENEKNPLAKYFEETGQYLTKYAKHDNGPA
IHKIKYYGNKLVEHLDITKNYHNPQNKVVQLSQKSFRFDVYQTDKGYKFISIAYLTLKNEKN
YYAISQEKYDQLKSEKKISNNAVFIGSFYTSDIIEINNEKFRVIGVNSDKNNLIEVDRIDIRQKEF
IELEEEKKNNRIKVTIGRKTTNIEKFHTDILGNMYKSKRPKAPQLVFKKG

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 730 (designated herein as Staphylococcus pasteuri Cas9 or Spa Cas9):

MKEKYILGLDLGITSVGYGIINFETKKIIDAGVRLFPEANVDNNEGRRSKRGSRRLKRRRIHRL
ERVKLLLTEYDLINKEQIPTSNNPYQIRVKGLSEILSKDELAIALLHLAKRRGIHNINVSSEDED
ASNELSTKEQINRNNKLLKDKYVCEVQLQRLKEGQIRGEKNRFKTTDILKEIDQLLKVQKDY
HNLDIDFINQYKEIVETRREYFEGPGQGSPFGWNGDLKKWYEMLMGHCTYFPQELRSVKYA
YSADLFNALNDLNNLIIQRDNSEKLEYHEKYHIIENVFKQKKKPTLKQIAKEIGVNPEDIKGYR
ITKSGTPQFTEFKLYHDLKSIVFDKSILENEAILDQIAEILTIYQDEQSIKEELNKLPEILNEQDK
AEIAKLIGYNGTHRLSLKCIHLINEELWQTSRNQMEIFNYLNIKPNKVDLSEQNKIPKDMVND
FILSPVVKRTFIQSINVINKVIEKYGIPEDIIIELARENNSDDRKKFINNLQKKNEATRKRINEIIG
QTGNQNAKRIVEKIRLHDQQEGKCLYSLESIALMDLLNNPQNYEVDHIIPRSVAFDNSIHNKV
LVKQIENSKKGNRTPYQYLNSSDAKLSYNQFKQHILNLSKSKDRISKKKKDYLLEERDINKFE
VQKEFINRNLVDTRYATRELTSYLKAYFSANNMDVKVKTINGSFTNHLRKVWRFDKYRNHG
YKHHAEDALIIANADFLFKENKKLQNTNKILEKPTIENNTKKVTVEKEEDYNNVFETPKLVED
IKQYRDYKFSHRVDKKPNRQLINDTLYSTRMKDEHDYIVQTITDIYGKDNTNLKKQFNKNPE
KFLMYQNDPKTFEKLSIIMKQYSDEKNPLAKYYEETGEYLTKYSKKNNGPIVKKIKLLGNKV
GNHLDVTNKYENSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYDIKYKDYCEINNIKG
EPRIKKTIGKKTESIEKFTTDVLGNLYLHSTEKAPQLIFKRGL

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 710 (designated herein as SpCas9):

MDKKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGD.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 731 (designated herein as Cas12i1):

MSNKEKNASETRKAYTTKMIPRSHDRMKLLGNFMDYLMDGTPIFFELWNQFGGGIDRDIISG
TANKDKISDDLLLAVNWFKVMPINSKPQGVSPSNLANLFQQYSGSEPDIQAQEYFASNFDTE
KHQWKDMRVEYERLLAELQLSRSDMHHDLKLMYKEKCIGLSLSTAHYITSVMFGTGAKNN
RQTKHQFYSKVIQLLEESTQINSVEQLASIILKAGDCDSYRKLRIRCSRKGATPSILKIVQDYEL
GTNHDDEVNVPSLIANLKEKLGRFEYECEWKCMEKIKAFLASKVGPYYLGSYSAMLENALS
PIKGMTTKNCKFVLKQIDAKNDIKYENEPFGKIVEGFFDSPYFESDTNVKWVLHPHHIGESNI
KTLWEDLNAIHSKYEEDIASLSEDKKEKRIKVYQGDVCQTINTYCEEVGKEAKTPLVQLLRY
LYSRKDDIAVDKIIDGITFLSKKHKVEKQKINPVIQKYPSFNFGNNSKLLGKIISPKDKLKHNL
KCNRNQVDNYIWIEIKVLNTKTMRWEKHHYALSSTRFLEEVYYPATSENPPDALAARFRTKT
NGYEGKPALSAEQIEQIRSAPVGLRKVKKRQMRLEAARQQNLLPRYTWGKDFNINICKRGN
NFEVTLATKVKKKKEKNYKVVLGYDANIVRKNTYAAIEAHANGDGVIDYNDLPVKPIESGF
VTVESQVRDKSYDQLSYNGVKLLYCKPHVESRRSFLEKYRNGTMKDNRGNNIQIDFMKDFE
AIADDETSLYYFNMKYCKLLQSSIRNHSSQAKEYREEIFELLRDGKLSVLKLSSLSNLSFVMF
KVAKSLIGTYFGHLLKKPKNSKSDVKAPPITDEDKQKADPEMFALRLALEEKRLNKVKSKKE
VIANKIVAKALELRDKYGPVLIKGENISDTTKKGKKSSTNSFLMDWLARGVANKVKEMVM
MHQGLEFVEVNPNFTSHQDPFVHKNPENTFRARYSRCTPSELTEKNRKEILSFLSDKPSKRPT
NAYYNEGAMAFLATYGLKKNDVLGVSLEKFKQIMANILHQRSEDQLLFPSRGGMFYLATYK
LDADATSVNWNGKQFWVCNADLVAAYNVGLVDIQKDFKKK

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 732 (designated herein as Cas12i2):

MSSAIKSYKSVLRPNERKNQLLKSTIQCLEDGSAFFFKMLQGLFGGITPEIVRFSTEQEKQQQD
IALWCAVNWFRPVSQDSLTHTIASDNLVEKFEEYYGGTASDAIKQYFSASIGESYYWNDCRQ
QYYDLCRELGVEVSDLTHDLEILCREKCLAVATESNQNNSIISVLFGTGEKEDRSVKLRITKKI
LEAISNLKEIPKNVAPIQEIILNVAKATKETFRQVYAGNLGAPSTLEKFIAKDGQKEFDLKKLQ
TDLKKVIRGKSKERDWCCQEELRSYVEQNTIQYDLWAWGEMFNKAHTALKIKSTRNYNFA
KQRLEQFKEIQSLNNLLVVKKLNDFFDSEFFSGEETYTICVHHLGGKDLSKLYKAWEDDPAD
PENAIVVLCDDLKNNFKKEPIRNILRYIFTIRQECSAQDILAAAKYNQQLDRYKSQKANPSVL
GNQGFTWTNAVILPEKAQRNDRPNSLDLRIWLYLKLRHPDGRWKKHHIPFYDTRFFQEIYAA
GNSPVDTCQFRTPRFGYHLPKLTDQTAIRVNKKHVKAAKTEARIRLAIQQGTLPVSNLKITEIS
ATINSKGQVRIPVKFDVGRQKGTLQIGDRFCGYDQNQTASHAYSLWEVVKEGQYHKELGCF
VRFISSGDIVSITENRGNQFDQLSYEGLAYPQYADWRKKASKFVSLWQITKKNKKKEIVTVE
AKEKFDAICKYQPRLYKFNKEYAYLLRDIVRGKSLVELQQIRQEIFRFIEQDCGVTRLGSLSLS
TLETVKAVKGIIYSYFSTALNASKNNPISDEQRKEFDPELFALLEKLELIRTRKKKQKVERIAN
SLIQTCLENNIKFIRGEGDLSTTNNATKKKANSRSMDWLARGVFNKIRQLAPMHNITLFGCGS
LYTSHQDPLVHRNPDKAMKCRWAAIPVKDIGDWVLRKLSQNLRAKNIGTGEYYHQGVKEF
LSHYELQDLEEELLKWRSDRKSNIPCWVLQNRLAEKLGNKEAVVYIPVRGGRIYFATHKVAT
GAVSIVFDQKQVWVCNADHVAAANIALTVKGIGEQSSDEENPDGSRIKLQLTS

### C. Determination of Efficacy of Guide RNAs

In some embodiments, the efficacy of a guide RNA is determined when delivered or

expressed together with other components forming an RNP. In some embodiments, the guide RNA is expressed together with an RNA-targeted endonuclease, including but not limited to any of the known Cas proteins and their variants known in the art and/or disclosed herein (e.g., SpCas9, SaCas9, SluCas9 or variants thereof). In some embodiments, the guide RNA is delivered to or expressed in a cell line that already stably expresses a the RNA-targeted endonuclease (e.g., Cas protein). In some embodiments the guide RNA is delivered to a cell as part of an RNP. In some embodiments, the guide RNA is delivered to a cell along with a nucleic acid (e.g., mRNA) encoding the RNA-targeted endonuclease (e.g., Cas protein).

In some embodiments, the efficacy of particular guide RNAs is determined based on in vitro models. In some embodiments, the in vitro model is a cell line.

In some embodiments, the efficacy of particular guide RNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of particular guide RNAs is determined based on in vivo models. In some embodiments, the in vivo model is a rodent model. In some embodiments, the rodent model is a mouse which expresses a gene comprising an expanded trinucleotide repeat or a self-complementary region. The gene may be the human version or a rodent (e.g., murine) homolog of any of the genes described throughout this application. In some embodiments, the gene is human DMPK. In some embodiments, the gene is a rodent (e.g., murine) homolog of DMPK In some embodiments, the in vivo model is anon-human primate, for example cynomolgus monkey. See, e.g., the mouse model described in Huguet et al., 2012, PLoS Genet, 8(11):e1003043. In some embodiments, the in vivo model is a non-human primate, for example cynomolgus monkey.

### III. Methods and Uses

Provided herein are methods and uses for gene editing, comprising contacting a cell with one or more guide RNAs or nucleic acids encoding one or more guide RNAs; and an RNA-targeted endonuclease, or a nucleic acid encoding the RNA-targeted endonuclease, wherein the guide RNA comprises a guide sequence that directs the RNA-targeted endonuclease to or near one or more regulatory elements that at least partially drive expression of a gene, and wherein the method causes disruption and/or exision of at least a portion of the one or more regulatory elements. In some embodiments, the disruption and/or exision of the one or more regulatory elements results in a reduction in production of a toxic protein or transcript.

Also provided are methods of treating a disease or disorder characterized by toxic proteins and transcripts in a human subject, comprising administering
a. one or more guide RNAs or nucleic acids encoding one or more guide RNAs; and
b. an RNA-targeted endonuclease, or a nucleic acid encoding the RNA-targeted endonuclease,
   wherein the guide RNA comprises a guide sequence that directs the RNA-targeted endonuclease to or near one or more regulatory elements that at least partially drive expression of a gene causing production of a toxic protein and/or toxic transcript, and wherein the method causes disruption and/or exision of at least a portion of the one or more regulatory elements, wherein the disruption and/or exision results in a reduction in the production of the toxic protein and/or transcript.

Also provided herein are methods of excising a regulatory element that at least partially drives expression of a gene that produces a toxic protein and/or toxic transcript. In some embodiments, the regulatory element is an intron. In a specific embodiment, the regulatory element is intron 1 of the DMPK gene. In some embodiments, the regulatory element is a transcription start site (TSS). In some embodiments, the regulatory element is an intronic E-box site. In some embodiments, the regulatory element is in exon 1 of the DMPK gene. In some embodiments, the regulatory element is intron 1 and exon 1 of the DMPK gene. In some embodiments, full or partial intron 1 of DMPK is excised along with full or partial exon 1 of DMPK.

In some embodiments, the gene is selected from: DM1 protein kinase (DMPK), CCHC-type zinc finger nucleic acid binding protein (CNBP), transcription Factor 4 (TCF4), junctophilin 3 (JPH3), huntingtin (HTT), ataxin 1 (ATXN1), ataxin 2 (ATXN2), ataxin 3 (ATXN3), ataxin 7 (ATXN7), ATXN8 opposite strand lncRNA (ATXN8OS), androgen receptor (AR), atrophin 1 (ATN1), fragile X Mental Retardation 1 (FMR1), chromosome 9 open reading frame 72 (C90RF72), polyadenylate-binding protein 2 (PABP2), amyloid precursor protein (APP), presenilin 1 (PSEN1), presenilin 2 (PSEN2), apolipoprotein E4 (APOE4), and synuclein alpha (SNCA).

In some embodiments, the region of interest for a respective gene is as follows:

TABLE 1

| Gene target | Potential Region of Interest 1 | Potential Region of Interest 2 |
|---|---|---|
| CNBP (DM2) | chr3: 129,173,132-129,187,485 | |
| HTT gene | chr4: 3,070,556-3,087,781 | chr4: 3,105,101-3,126,261 |
| C9ORF72 | chr9: 27,572,203-27,574,934 | |
| ATXN1 | chr6: 16,631,111-16,862,285 | |
| ATXN2 | chr12: 111,555,689-111,604,722 | |
| ATXN3 | chr14: 92,101,625-92,111,230 | |
| ATXN7 | chr3: 63,846,443-63,986,038 | |
| ATXN8 | chr13: 70,101,070-70,115,282 | |
| FMR1 | chrX: 147,910,266-147,914,185 | |
| APP | chr21: 26,021,307-26,214,787 | chr21: 25,825,845-26,040,823 |
| TCF4 | chr18: 55,231,638-55,781,081 | |
| JPH3 | chr16: 87,576,271-87,608,153 | |
| androgen receptor (AR) | chrX: 67,505,518-67,629,916 | |

Provided herein also are methods and uses for ameliorating or treating certain diseases and disorders characterized by toxic or aberrant mRNA, DNA, and/or protein production (toxic proteins and transcripts) by administering one or more guide RNAs or nucleic acids encoding one or more guide RNAs; and an RNA-targeted endonuclease, or a nucleic acid encoding the RNA-targeted endonuclease, wherein the guide RNA comprises a guide sequence that directs the RNA-targeted endonuclease to or near one or more regulatory elements that at least partially drive expression of a gene, and wherein the method causes disruption and/or exision of at least a portion of the one or more regulatory elements. Treating any such disease or disorder characterized by toxic proteins and/or transcripts described herein may comprises alleviating the symptoms of the disease or disorder. In some embodiments, the gene is DM1 protein kinase (DMPK) and the disorder is DM1. In some embodiments, the gene is CCHC-type zinc finger nucleic acid binding protein (CNBP) and the disease is DM2. In some embodiments, the gene is transcription Factor 4 (TCF4) and the disease is Fuchs endothelial corneal dystrophy. In some embodiments, the gene is junctophilin 3 (JPH3) and the disease is Huntington disease-like 2. In some embodiments, the gene is huntingtin (HTT) and the disease is Huntington disease. In some embodiments, the gene is ataxin 1 (ATXN1) and the disease is spinocerebellar ataxia type 1. In some embodiments, the gene is ataxin 2 (ATXN2) and the disease is spinocerebellar ataxia type 2. In some embodiments, the gene is ataxin 3 (ATXN3) and the disease is spinocerebellar ataxia type 3. In some embodiments, the gene is ataxin 7 (ATXN7) and the disease is spinocerebellar ataxia type 7. In some embodiments, the gene is ATXN8 opposite strand lncRNA (ATXN8OS) and the disease is spinocerebellar ataxia type 8. In some embodiments, the gene is androgen receptor (AR) and the disease is spinal and bulbar muscular atrophy. In some embodiments, the gene is atrophin 1 (ATN1) and the disease is dentatorubral-pallidoluysian atrophy. In some embodiments, the gene is fragile X Mental Retardation 1 (FMR1) and the disease is fragile X-associated tremor/ataxia syndrome. In some embodiments, the gene is chromosome 9 open reading frame 72 (C90RF72) and the disease is frontotemporal dementia or amyotrophic lateral sclerosis (ALS). In some embodiments, the gene is polyadenylate-binding protein 2 (PABP2) and the disease is oculopharyngeal muscular dystrophy. In some embodiments, the gene is amyloid precursor protein (APP), presenilin 1 (PSEN1), presenilin 2 (PSEN2), or apolipoprotein E4 (APOE4) and the disease is Alzheimer's disease. In some embodiments, the gene is synuclein alpha (SNCA) and the disease is Parkinson's disease.

In some embodiments, if the gene is DMPK and a subject has myotonic dystrophy type 1 (DM1), then administration to the subject leads to treatment of DM1. In some embodiments, if the gene is CNBP and a subject has myotonic dystrophy type 2 (DM2), then administration to the subject leads to treatment of DM2. In some embodiments, if the gene is TCF4 and a subject has Fuchs endothelial corneal dystrophy (FECD), then administration to the subject leads to treatment of FECD. In some embodiments, if the gene is JPH3 and a subject has Huntington disease-like 2 (HDL2), then administration to the subject leads to treatment of HDL2. In some embodiments, if the gene is HTT and a subject has Huntington disease (HD), then administration to the subject leads to treatment of HD. In some embodiments, if the gene is ATXN and a subject has Spinocerebellar ataxia (SCA), then administration to the subject leads to treatment of SCA. In some embodiments, if the gene is AR and a subject has Spinal and bulbar muscular atrophy (SBMA), then administration to the subject leads to treatment of SBMA. In some embodiments, if the gene is ATN1 and a subject has Dentatorubral-pallidoluysian atrophy (DRPLA), then administration to the subject leads to treatment of DRPLA. In some embodiments, if the gene is FMR1 and a subject has Fragile X-associated tremor/ataxia syndrome (FXTAS), then administration to the subject leads to treatment of FXTAS. In some embodiments, if the gene is C900RF72 and a subject has Frontotemporal dementia (FTD) or amyotrophic lateral sclerosis (ALS), then administration to the subject leads to treatment of FTD or ALS. In some embodiments, if the gene is PABP2 and a subject has Oculopharyngeal muscular dystrophy (OMPD), then administration to the subject leads to the treatment of OPMD. In some embodiments, if the gene is APP, PSEN1, PSEN2, or APOE4, and a subject has Alzheimer's Disease (AD), then administration to the subject leads to treatment of AD. In some embodiments, if the gene is SNCA and a subject has Parkinson's Disease (PD), then administration to the subject leads to treatment of PD.

In some embodiments, the gene is associated with aberrant nucleotide repeats. In some embodiments, the aberrant nucleotide repeat is a CTG repeat, a CAG repeat, a GGGGCC repeat, or GC(N) repeat. In some embodiments, the aberrant nucleotides repeat is a trinucleotide repeat. In some embodiments, the at least two guide RNAs or pair of guide RNAs together target sequences that disrupt a regulatory region of a gene characterized by a repeat region. In some embodiments, the guide RNA excises at least a portion of a regulatory region that at least partially drives expression of a gene. In some embodiments, the guide RNAs do not flank the repeat region. In some embodiments, the guide RNAs do not excise nucleotide repeats. In some embodiments, the gene is not associated with aberrant nucleotide repeats.

In some embodiments, at least two guide RNAs (e.g., a pair of guide RNAs) direct an RNA-targeted endonuclease to or near a regulatory element. In some embodiments, at least one of the guide RNAs directs the RNA-targeted endonuclease to a target sequence within 5, within 10, within 20, within 30, within 40, within 50 or within 100 nucleotides of the regulatory element.

In some embodiments, the regulatory element is not in the exon of the target gene. In some embodiments, the regulatory element is in an intron and the one or more guide RNAs direct the RNA-targeted endonuclease to a target sequence in the same intron. In some embodiments, the regulatory element is an enhancer. In some embodiments, the regulatory element is a promoter.

In some embodiments, the regulatory element is a transcription start site (TSS), E-box, NF-κB binding domain, 20 bp direct repeats, transcription factor AP2, transcription factor Sp1, Huntington disease gene regulatory region-binding protein 1 and 2 (HDBP1 and HDBP2), uncharacterized regulatory region (UCRR), 22-bp methylation sensitive element (MSE), and/or a single-nucleotide polymorphism (SNP) variant comprising a T/C at ss71651738. In some embodiments, the regulatory element is a TSS and E-box. In some embodiments, the regulatory element is selected from NF-κB binding domain, 20 bp direct repeats, transcription factor AP2, transcription factor Sp1, Huntington disease gene regulatory region-binding protein 1 and 2 (HDBP1 and HDBP2), uncharacterized regulatory region (UCRR). In another embodiment, the regulatory element is selected from 2-bp methylation sensitive element (MSE), and a single-nucleotide polymorphism (SNP) variant comprising a T/C at ss71651738. In some embodiments, the method comprises at least two guide RNAs, wherein the guide RNAs direct the RNA-targeted endonuclease to or near an E-box and/or a TSS.

In some embodiments, the method reduces expression of a target gene by at least 10%, 20%, 30%, 40%, 50% 60%, 70%, 80%, 90%, 95%, 96%, 97%, 98%, 99%, or 100%. In some embodiments, the method does not eliminate expression of the target gene.

### 1. DMPK

In some embodiments, the gene is DMPK and the one or more regulatory elements is selected from a transcription start site (TSS) and an E-Box. In some embodiments, the regulatory element is a TSS. In some embodiments, the regulatory element is an E-box. In some embodiments, the method comprises the use of at least two guide RNAs, wherein the guide RNAs direct the RNA-targeted endonuclease to or near an E-box and/or a TSS. In some embodiments, no portion of the target gene that codes for the gene is excised. In some embodiments, a portion of the exon is excised. In some embodiments, the entire exon is excised. In some embodiments, a portion or the entirety of an intron and a portion or the entirety of an exon is excised. In some embodiments, the method does not completely eliminate expression of the target gene. In some embodiments, the method reduces expression of the target gene by at least 10%, 20%, 30%, 40%, or 50%. In some embodiments, the reduction of DMPK transcript level is at least 50%, 60%, 70%, 80%, 90%, 95%, 96%, 97%, 98%, 99%, or 100%. In some embodiments, the reduction of DMPK transcript level is at least 70%. In some embodiments, the method abolishes CUG RNA foci in about 30%, about 40%, or about 50% myoblast nuclei. In some embodiments, the method ameliorates or treats Myotonic Dystrophy Type 1 (DM1). In some embodiments, an entire intron and an entire exon are excised. In some embodiments, at least a portion of exon 1 is excised from DMPK. In some embodiments, at least a portion of intron 1 is excised from DMPK. In some embodiments, at least a portion of exon 1 and intron 1 are excised from DMPK.

### 2. CNBP

In some embodiments, the gene is CNBP and at least one regulatory element that at least partially drives expression of CNBP is targeted by one or more guide RNAs. In some embodiments, a region of interest for gene editing occurs at about: chr3:129,173,132-129,187,485. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of CNBP transcript level is at least 50%, 60%, 70%, 80%, 90%, 95%, 96%, 97%, 98%, 99%, or 100%. In some embodiments, the reduction of CNBP transcript level is at least 70%. In some embodiments, the method abolishes CCTG RNA foci in about 30%, about 40%, or about 50% myoblast nuclei. In some embodiments, the method ameliorates or treats Myotonic dystrophy type 2 (DM2).

### 3. TCF4

In some embodiments, the gene is TCF4 and at least one regulatory element that at least partially drives expression of TCF4 is targeted by one or more guide RNAs. In some embodiments, a region of interest for gene editing occurs at about: chr18:55,231,638-55,781,081. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of TCF4 transcript level is at least 50%, 60%, 70%, 80%, 90%, 95%, 96%, 97%, 98%, 99%, or 100%. In some embodiments, the reduction of TCF4 transcript level is at least 70%. In some embodiments, the method abolishes CTG RNA foci in about 30%, about 40%, or about 50% myoblast nuclei. In some embodiments, the method ameliorates or treats Fuchs endothelial corneal dystrophy (FECD).

### 4. JPH3

In some embodiments, the gene is JPH3 and at least one regulatory element that at least partially drives expression of JPH3 is targeted by one or more guide RNAs. In some embodiments, a region of interest for gene editing occurs at about: chr16:87,576,271-87,608,153. In some embodiments, no portion of the target gene that codes for the genes is excised. In some embodiments, the method does not completely eliminate expression of the target gene. In some embodiments, the method reduces expression of the target gene by at least 10%, 20%, 30%, 40%, or 50%. In some embodiments, the reduction of JPH3 transcript level is at least 50%, 60%, 70%, 80%, 90%, 95%, 96%, 97%, 98%, 99%, or 100%. In some embodiments, the reduction of JPH3 transcript level is at least 70%. In some embodiments, the method abolishes CTG RNA foci in about 30%, about 40%, or about 50% myoblast nuclei. In some embodiments, the method ameliorates or treats Huntington disease-like 2 (HDL2).

### 5. HTT

In some embodiments, the gene is HTT and the one or more regulatory elements is selected from NF-κB binding domain, 20 bp direct repeats, transcription factor AP2, transcription factor Sp1, Huntington disease gene regulatory region-binding protein 1 and 2 (HDBP1 and HDBP2), and uncharacterized regulatory region (UCRR). In some embodiments, a region of interest for gene editing occurs at about: chr4:3,070,556-3,087,781. In some embodiments, a region of interest for gene editing occurs at about: chr4:3,105,101-3,126,261. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of HTT transcript level is at least 50%, 60%, 70%, 80%, 90%, 95%, 96%, 97%, 98%, 99%, or 100%. In some embodiments, the reduction of HTT transcript level is at least 70%. In some embodiments, the method ameliorates or treats Huntington disease (HD).

### 6. ATXN1

In some embodiments, the gene is ANTXN1 and at least one regulatory element that at least partially drives expression of ANTXN1 is targeted by one or more guide RNAs. In some embodiments, a region of interest for gene editing occurs at about: chr6:16,631,111-16,862,285. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of ANTXN1 transcript level is at least 50%, 60%, 70%, 80%, 90%, 95%, 96%, 97%, 98%, 99%, or 100%. In some embodiments, the reduction of ANTXN1 transcript level is at least 70%. In some embodiments, the method ameliorates or treats Spinocerebellar ataxia (SCA).

### 7. ATXN2

In some embodiments, the gene is ANTXN2 and at least one regulatory element that at least partially drives expression of ANTXN2 is targeted by one or more guide RNAs. In some embodiments, a region of interest for gene editing occurs at about: chr12:111,555,689-111,604,722. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of ANTXN2 transcript level is at least 50%, 60%, 70%, 80%, 90%, 95%, 96%, 97%, 98%, 99%, or 100%. In some embodiments, the reduction of ANTXN2 transcript level is at least 70%. In some embodiments, the method ameliorates or treats Spinocerebellar ataxia (SCA).

### 8. ATXN3

In some embodiments, the gene is ANTXN3 and at least one regulatory element that at least partially drives expression of ANTXN3 is targeted by one or more guide RNAs. In some embodiments, a region of interest for gene editing occurs at about: chr14:92,101,625-92,111,230. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of ANTXN3 transcript level is at least 50%, 60%, 70%, 80%, 90%, 95%, 96%, 97%, 98%, 99%, or 100%. In some embodiments, the reduction of ANTXN3 transcript level is at least 70%. In some embodiments, the method ameliorates or treats Spinocerebellar ataxia (SCA).

### 9. ATXN7

In some embodiments, the gene is ANTXN7 and at least one regulatory element that at least partially drives expression of ANTXN7 is targeted by one or more guide RNAs. In some embodiments, a region of interest for gene editing occurs at about: chr3:63,846,443-63,986,038. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of ANTXN7 transcript level is at least 50%, 60%, 70%, 80%, 90%, 95%, 96%, 97%, 98%, 99%, or 100%. In some embodiments, the reduction of ANTXN7 transcript level is at least 70%. In some embodiments, the method ameliorates or treats Spinocerebellar ataxia (SCA).

### 10. ATXN8

In some embodiments, the gene is ANTXN8 and at least one regulatory element that at least partially drives expression of ANTXN8 is targeted by one or more guide RNAs. In some embodiments, a region of interest for gene editing occurs at about: chr13:70,101,070-70,115,282. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of ANTXN8 transcript level is at least 50%, 60%, 70%, 80%, 90%, 95%, 96%, 97%, 98%, 99%, or 100%. In some embodiments, the reduction of ANTXN8 transcript level is at least 70%. In some embodiments, the method ameliorates or treats Spinocerebellar ataxia (SCA).

### 11. ATXN8OS

In some embodiments, the gene is ANTXN8OS and at least one regulatory element that at least partially drives expression of ANTXN8OS is targeted by one or more guide RNAs. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of ANTXN8OS transcript level is at least 50%, 60%, 70%, 80%, 90%, 95%, 96%, 97%, 98%, 99%, or 100%. In some embodiments, the reduction of ANTXN8OS transcript level is at least 70%. In some embodiments, the method ameliorates or treats Spinocerebellar ataxia (SCA).

### 12. AR

In some embodiments, the gene is AR and at least one regulatory element that at least partially drives expression of AR is targeted by one or more guide RNAs. In some embodiments, a region of interest for gene editing occurs at about: chrX:67,505,518-67,629,916. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of AR transcript level is at least 50%, 60%, 70%, 80%, 90%, 95%, 96%, 97%, 98%, 99%, or 100%. In some embodiments, the reduction of AR transcript level is at least 70%. In some embodiments, the method ameliorates or treats Spinal and bulbar muscular atrophy (SBMA).

### 13. ATN1

In some embodiments, the gene is ANT1 and at least one regulatory element that at least partially drives expression of ANT1 is targeted by one or more guide RNAs. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of ANT1 transcript level is at least 50%, 60%, 70%, 80%, 90%, 95%, 96%, 97%, 98%, 99%, or 100%. In some embodiments, the reduction of ANT1 transcript level is at least 70%. In some embodiments, the method ameliorates or treats Dentatorubral-pallidoluysian atrophy (DRPLA).

### 14. FMR1

In some embodiments, the gene is FMR1 and the one or more regulatory elements is selected from 2-bp methylation sensitive element (MSE) and the single-nucleotide polymorphism (SNP) variant comprising a T/C at ss71651738. In some embodiments, a region of interest for gene editing occurs at about: chrX:147,910,266-147,914,185. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of FMR1 transcript level is at least 50%, 60%, 70%, 80%, 90%, 95%, 96%, 97%, 98%, 99%, or 100%. In some embodiments, the reduction of FMR1 transcript level is at least 70%. In some embodiments, the method ameliorates or treats Fragile X-associated tremor/ataxia syndrome (FXTAS).

### 15. C90RF72

In some embodiments, the gene is C90RF72 and at least one regulatory element that at least partially drives expression of C900RF72 is targeted by one or more guide RNAs. In some embodiments, a region of interest for gene editing occurs at about: chr9:27,572,203-27,574,934. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of C90RF72 transcript level is at least 50%, 60%, 70%, 80%, 90%, 95%, 96%, 97%, 98%, 99%, or 100%. In some embodiments, the reduction of C90RF72 transcript level is at least 70%. In some embodiments, the method ameliorates or treats Frontotemporal dementia (FTD) or amyotrophic lateral sclerosis (ALS).

### 16. PABP2

In some embodiments, the gene is PABP2 and at least one regulatory element that at least partially drives expression of PABP2 is targeted by one or more guide RNAs. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of PABP2 transcript level is at least 50%, 60%, 70%, 80%, 90%, 95%, 96%, 97%, 98%, 99%, or 100%. In some embodiments, the reduction of PABP2 transcript level is at least 70%. In some embodiments, the method ameliorates or treats Oculopharyngeal muscular dystrophy (OPMD).

### 17. APP

In some embodiments, the gene is APP and at least one regulatory element that at least partially drives expression of APP is targeted by one or more guide RNAs. In some embodiments, a region of interest for gene editing occurs at about: chr21:26,021,307-26,214,787. In some embodiments, a region of interest for gene editing occurs at about: chr21:25,825,845-26,040,823. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of APP transcript level is at least 50%, 60%, 70%, 80%, 90%, 95%, 96%, 97%, 98%, 99%, or 100%. In some embodiments, the reduction of APP transcript level is at least 70%. In some embodiments, the method ameliorates or treats AD.

### 18. PSEN1

In some embodiments, the gene is PSEN1 and at least one regulatory element that at least partially drives expression of PSEN1 is targeted by one or more guide RNAs. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of PSEN1 transcript level is at least 50%, 60%, 70%, 80%, 90%, 95%, 96%, 97%, 98%, 99%, or 100%. In some embodiments, the reduction of PSEN1 transcript level is at least 70%. In some embodiments, the method ameliorates or treats Alzheimer's Disease (AD).

### 19. PSEN2

In some embodiments, the gene is PSEN2 and at least one regulatory element that at least partially drives expression of PSEN2 is targeted by one or more guide RNAs. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of PSEN2 transcript level is at least 50%, 60%, 70%, 80%, 90%, 95%, 96%, 97%, 98%, 99%, or 100%. In some embodiments, the reduction of PSEN2 transcript level is at least 70%. In some embodiments, the method ameliorates or treats Alzheimer's Disease (AD).

### 20. APOE4

In some embodiments, the gene is APOE4 and at least one regulatory element that at least partially drives expression of APOE4 is targeted by one or more guide RNAs. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of APOE4 transcript level is at least 50%, 60%, 70%, 80%, 90%, 95%, 96%, 97%, 98%, 99%, or 100%. In some embodiments, the reduction of APOE4 transcript level is at least 70%. In some embodiments, the method ameliorates or treats Alzheimer's Disease (AD).

### 21. SNCA

In some embodiments, the gene is SNCA and at least one regulatory element that at least partially drives expression of SNCA is targeted by one or more guide RNAs. In some embodiments, no portion of the target gene that codes for the gene is excised. In some embodiments, the method does not completely eliminate expression of the target gene. In some embodiments, the method reduces expression of the target gene by at least 10%, 20%, 30%, 40%, or 50%. In some embodiments, the reduction of SNCA transcript level is at least 50%, 60%, 70%, 80%, 90%, 95%, 96%, 97%, 98%, 99%, or 100%. In some embodiments, the reduction of SNCA transcript level is at least 70%. In some embodiments, the method ameliorates or treats Parkinson's Disease (PD).

In the methods described herein, the guide RNA may be expressed together with an RNA-targeted endonuclease, including but not limited to any of the Cas proteins known in the art and/or disclosed herein (e.g., SpCas9, SaCas9, SluCas9, ShyCas9, SmiCas9, SpaCas9, Cas12i1, Cas12i2, sRGN1, sRGN2, sRGN3, sRGN3.1, sRGN3.2, sRGN3.3, sRGN4). In some embodiments, the RNA-targeted endonuclease is a Cas nuclease or Cpf1 nuclease. In some embodiments, the Cas nuclease is Cas9. In some embodiments, the Cas9 nuclease is from Streptococcus pyogenes (spCas9), Staphylococcus aureus (saCas9), or Staphylococcus lugdunensis (sluCas9). In some embodiments, the Cas nuclease is sRGN3.1. In some embodiments, the Cas nuclease is sRGN3.3. In some embodiments, the Cas protein is not a Cas nickase. In some embodiments, the Cas protein is not a Cas nickase and CRISPR interference is not used. In particular embodiments, the Cas protein is not a nuclease-deficient/nuclease-dead Cas protein.

In some embodiments, the guide RNA is an sgRNA. In some embodiments, the sgRNA is modified, wherein the modification alters one or more 2′ positions and/or phosphodiester linkages. In some embodiments, the modification alters one or more, or all, of the first three nucleotides of the sgRNA and/or of the last three nucleotides of the sgRNA. In some embodiments, the modification includes one or more of a phosphorothioate modification, a 2′-OMe modification, a 2′-O-MOE modification, a 2′-F modification, a 2′-O-methine-4′ bridge modification, a 3′-thiophosphonoacetate modification, or a 2′-deoxy modification.

In some embodiments, the guide RNA is associated with a lipid nanoparticle (LNP) or a viral vector to deliver the guide RNAs and compositions therein. In some embodiments, the vectors or LNPs associated with the guide RNAs and compositions therein are for use in preparing a medicament or pharmaceutical formulation for treating a disease or disorder. In some embodiments, the viral vector is an adeno-associated virus (AAV) vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. The AAV vector can be an AAV1, AAV2, AAV3, AAV4, AAV5, AAV6, AAV7, AAV8, AAVrh10, AAVrh74, or AAV9 vector, where the number following AAV indicates the AAV serotype. In some embodiments, the AAV vector is an AAV serotype 9 vector, an AAVrh10 vector, or an AAVrh74 vector. In some embodiments, the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.

In some embodiments of the methods described herein, at least two guide RNAs act as a pair to excise at least a portion of a regulatory element that at least partially drives expression of a gene.

Further provided herein is a method and use for treating DM1 in a human subject, comprising administering one or more guide RNAs or nucleic acids encoding one or more guide RNAs; and an RNA-targeted endonuclease, or a nucleic acid encoding the RNA-targeted endonuclease, wherein the guide RNA comprises a guide sequence that directs the RNA-targeted endonuclease to or near one or more regulatory elements that at least partially drive expression of the DMPK gene, and wherein the method causes disruption and/or exision of at least a portion of the one or more regulatory elements.

In some method and use embodiments, where the regulatory element is TSS, a pair of guide RNAs is administered, wherein the pair of guide RNAs comprises a first guide RNA comprising at least 17, 18, 19, or 20 contiguous nucleotides of a first guide sequence selected from any one of SEQ ID NOs: 1, 2, and 3, and a second guide RNA comprising at least 17, 18, 19, or 20 contiguous nucleotides of a second guide sequence selected from any one of SEQ ID NOs: 4, 5, and 6. In some embodiments, the pair of guide RNAs comprises:
a. a first guide RNA comprising at least 17, 18, 19, or 20 contiguous nucleotides of a first guide sequence of SEQ ID NO: 1 and a second guide RNA comprising at least 17, 18, 19, or 20 contiguous nucleotides of a second guide sequence of SEQ ID NO: 4;
b. a first guide RNA comprising at least 17, 18, 19, or 20 contiguous nucleotides of a first guide sequence of SEQ ID NO: 1 and a second guide RNA comprising at least 17, 18, 19, or 20 contiguous nucleotides of a second guide sequence of SEQ ID NO: 5;
c. a first guide RNA comprising at least 17, 18, 19, or 20 contiguous nucleotides of a first guide sequence of SEQ ID NO: 1 and a second guide RNA comprising at least 17, 18, 19, or 20 contiguous nucleotides of a second guide sequence of SEQ ID NO: 6;
d. a first guide RNA comprising at least 17, 18, 19, or 20 contiguous nucleotides of a first guide sequence of SEQ ID NO: 2 and a second guide RNA comprising at least 17, 18, 19, or 20 contiguous nucleotides of a second guide sequence of SEQ ID NO: 4;
e. a first guide RNA comprising at least 17, 18, 19, or 20 contiguous nucleotides of a first guide sequence of SEQ ID NO: 2 and a second guide RNA comprising at least 17, 18, 19, or 20 contiguous nucleotides of a second guide sequence of SEQ ID NO: 5;
f. a first guide RNA comprising at least 17, 18, 19, or 20 contiguous nucleotides of a first guide sequence of SEQ ID NO: 2 and a second guide RNA comprising at least 17, 18, 19, or 20 contiguous nucleotides of a second guide sequence of SEQ ID NO: 6;
g. a first guide RNA comprising at least 17, 18, 19, or 20 contiguous nucleotides of a first guide sequence of SEQ ID NO: 3 and a second guide RNA comprising at least 17, 18, 19, or 20 contiguous nucleotides of a second guide sequence of SEQ ID NO: 4;
h. a first guide RNA comprising at least 17, 18, 19, or 20 contiguous nucleotides of a first guide sequence of SEQ ID NO: 3 and a second guide RNA comprising at least 17, 18, 19, or 20 contiguous nucleotides of a second guide sequence of SEQ ID NO: 5; or
i. a first guide RNA comprising at least 17, 18, 19, or 20 contiguous nucleotides of a first guide sequence of SEQ ID NO:3 and a second guide RNA comprising at least 17, 18, 19, or 20 contiguous nucleotides of a second guide sequence of SEQ ID NO: 6.

In some method and use embodiments, a composition is provided comprising a pair of guide RNAs targeting a TSS of the DMPK gene, wherein the pair of guide RNAs comprise a first guide RNA comprising a first guide sequence selected from any one of SEQ ID NOs: 1, 101, 102, 103, 2, 201, 202, 203, 3, 301, 302, and 303; and a second guide RNA comprising a second guide sequence selected from any one of SEQ ID NOs: 4, 401, 402, 403, 5, 501, 502, 503, 6, 601, 602, and 603. In some embodiments, the first guide RNA comprises a first guide sequence selected from any one of SEQ ID NOs: 1, 2, and 3; and the second guide RNA comprises a second guide sequence selected from any one of SEQ ID NOs: 4, 5, and 6. In some embodiments, the first guide RNA comprises a first guide sequence selected from any one of SEQ ID NOs: 101, 201, and 301; and the second guide RNA comprises a second guide sequence selected from any one of SEQ ID NOs: 401, 501, and 601. In some embodiments, the first guide RNA comprises a first guide sequence selected from any one of SEQ ID NOs: 102, 202, and 302; and the second guide RNA comprises a second guide sequence selected from any one of SEQ ID NOs: 402, 502, and 602. In some embodiments, the first guide RNA comprises a first guide sequence selected from any one of SEQ ID NOs: 103, 203, and 303; and the second guide RNA comprises a second guide sequence selected from any one of SEQ ID NOs: 403, 503, and 603.

In some method and use embodiments, the regulatory element is TSS and a pair of guide RNAs is administered, wherein the pair of guide RNAs comprise a first guide RNA comprising a first guide sequence that binds to a nucleotide sequence that is complementary to any one of SEQ ID NOs: 1, 2, and 3, and a second guide RNA comprising a second guide sequences that binds to a nucleotide sequence that is complementary to any one of SEQ ID NOs: 4, 5, and 6. In some embodiments, the pair of guide RNAs comprises:
a. a first guide RNA comprising a guide sequence that binds to a nucleotide sequence that is complementary to SEQ ID NO: 1 and a second guide RNA comprising a guide sequence that binds to a nucleotide sequence that is complementary to SEQ ID NO: 4;
b. a first guide RNA comprising a guide sequence that binds to a nucleotide sequence that is complementary to SEQ ID NO: 1 and a second guide RNA comprising a guide sequence that binds to a nucleotide sequence that is complementary to SEQ ID NO: 5;
c. a first guide RNA comprising a guide sequence that binds to a nucleotide sequence that is complementary to SEQ ID NO: 1 and a second guide RNA comprising a guide sequence that binds to a nucleotide sequence that is complementary to SEQ ID NO: 6;
d. a first guide RNA comprising a guide sequence that binds to a nucleotide sequence that is complementary to SEQ ID NO: 2 and a second guide RNA comprising a guide sequence that binds to a nucleotide sequence that is complementary to SEQ ID NO: 4;
e. a first guide RNA comprising a guide sequence that binds to a nucleotide sequence that is complementary to SEQ ID NO: 2 and a second guide RNA comprising a guide sequence that binds to a nucleotide sequence that is complementary to SEQ ID NO: 5;
f. a first guide RNA comprising a guide sequence that binds to a nucleotide sequence that is complementary to SEQ ID NO: 2 and a second guide RNA comprising a guide sequence that binds to a nucleotide sequence that is complementary to SEQ ID NO: 6;
g. a first guide RNA comprising a guide sequence that binds to a nucleotide sequence that is complementary to SEQ ID NO: 3 and a second guide RNA comprising a guide sequence that binds to a nucleotide sequence that is complementary to SEQ ID NO: 4;
h. a first guide RNA comprising a guide sequence that binds to a nucleotide sequence that is complementary to SEQ ID NO: 3 and a second guide RNA comprising a guide sequence that binds to a nucleotide sequence that is complementary to SEQ ID NO: 5; or
i. a first guide RNA comprising a guide sequence that binds to a nucleotide sequence that is complementary to SEQ ID NO: 3 and a second guide RNA comprising a guide sequence that binds to a nucleotide sequence that is complementary to SEQ ID NO: 6.

In some method and use embodiments, the regulatory element is an E-box and a pair of guide RNAs is administered, wherein the pair of guide RNAs comprise a first guide RNA comprising at least 17, 18, 19, or 20 contiguous nucleotides of a first guide sequence selected from any one of SEQ ID NOs: 7, 8, and 9, and a second guide RNA comprising at least 17, 18, 19, or 20 contiguous nucleotides of a second guide sequence selected from any one of SEQ ID NOs: 10, 11, and 12. In some embodiments, the pair of guide RNAs comprises:
a. a first guide RNA comprising at least 17, 18, 19, or 20 contiguous nucleotides of a first guide sequence of SEQ ID NO: 7 and a second guide RNA comprising at least 17, 18, 19, or 20 contiguous nucleotides of a second guide sequence of SEQ ID NO: 10;
b. a first guide RNA comprising at least 17, 18, 19, or 20 contiguous nucleotides of a first guide sequence of SEQ ID NO: 7 and a second guide RNA comprising at least 17, 18, 19, or 20 contiguous nucleotides of a second guide sequence of SEQ ID NO: 11;
c. a first guide RNA comprising at least 17, 18, 19, or 20 contiguous nucleotides of a first guide sequence of SEQ ID NO: 7 and a second guide RNA comprising at least 17, 18, 19, or 20 contiguous nucleotides of a second guide sequence of SEQ ID NO: 12;
d. a first guide RNA comprising at least 17, 18, 19, or 20 contiguous nucleotides of a first guide sequence of SEQ ID NO: 8 and a second guide RNA comprising at least 17, 18, 19, or 20 contiguous nucleotides of a second guide sequence of SEQ ID NO: 10;
e. a first guide RNA comprising at least 17, 18, 19, or 20 contiguous nucleotides of a first guide sequence of SEQ ID NO: 8 and a second guide RNA comprising at least 17, 18, 19, or 20 contiguous nucleotides of a second guide sequence of SEQ ID NO: 11;
f. a first guide RNA comprising at least 17, 18, 19, or 20 contiguous nucleotides of a first guide sequence of SEQ ID NO: 8 and a second guide RNA comprising at least 17, 18, 19, or 20 contiguous nucleotides of a second guide sequence of SEQ ID NO: 12;
g. a first guide RNA comprising at least 17, 18, 19, or 20 contiguous nucleotides of a first guide sequence of SEQ ID NO: 9 and a second guide RNA comprising at least 17, 18, 19, or 20 contiguous nucleotides of a second guide sequence of SEQ ID NO: 10;
h. a first guide RNA comprising at least 17, 18, 19, or 20 contiguous nucleotides of a first guide sequence of SEQ ID NO: 9 and a second guide RNA comprising at least 17, 18, 19, or 20 contiguous nucleotides of a second guide sequence of SEQ ID NO: 11; or
i. a first guide RNA comprising at least 17, 18, 19, or 20 contiguous nucleotides of a first guide sequence of SEQ ID NO: 9 and a second guide RNA comprising at least 17, 18, 19, or 20 contiguous nucleotides of a second guide sequence of SEQ ID NO: 12.

In some method and use embodiments, a composition is provided comprising a pair of guide RNAs targeting an E-box of the DMPK gene, wherein the pair of guide RNAs comprise a first guide RNA comprising a first guide sequence selected from any one of SEQ ID NOs: 7, 701, 702, 703, 8, 801, 802, 803, 9, 901, 902, and 903 and a second guide RNA comprising a second guide sequence selected from any one of SEQ ID NOs: 10, 1001, 1002, 1003, 11, 1101, 1102, 1103, 12, 1201, 1202, and 1203. In some embodiments, the first guide RNA comprises a first guide sequence selected from any one of SEQ ID NOs: 7, 8, and 9; and the second guide RNA comprises a second guide sequence selected from any one of SEQ ID NOs: 10, 11, and 12. In some embodiments, the first guide RNA comprises a first guide sequence selected from any one of SEQ ID NOs: 701, 801, and 901; and the second guide RNA comprises a second guide sequence selected from any one of SEQ ID NOs: 1001, 1101, and 1201. In some embodiments, the first guide RNA comprises a first guide sequence selected from any one of SEQ ID NOs: 702, 802, and 902; and the second guide RNA comprises a second guide sequence selected from any one of SEQ ID NOs: 1002, 1102, and 1202. In some embodiments, the first guide RNA comprises a first guide sequence selected from any one of SEQ ID NOs: 703, 803, and 903; and the second guide RNA comprises a second guide sequence selected from any one of SEQ ID NOs: 1003, 1103, and 1203.

In some method and use embodiments, the regulatory element is and E-box and a pair of guide RNAs is administered, wherein the pair of guide RNAs comprise a first guide RNA comprising a first guide sequence that binds to a nucleotide sequence that is complementary to any one of SEQ ID NOs: 7, 8, and 9, and a second guide RNA comprising a second guide sequences that binds to a nucleotide sequence that is complementary to any one of SEQ ID NOs: 10, 11, and 12. In some embodiments, the pair of guide RNAs comprises:
a. a first guide sequence that binds to a nucleotide sequence that is complementary to SEQ ID NO: 7 and a second guide sequence that binds to a nucleotide sequence that is complementary to SEQ ID NO: 10;
b. a first guide sequence that binds to a nucleotide sequence that is complementary to SEQ ID NO: 7 and a second guide sequence that binds to a nucleotide sequence that is complementary to SEQ ID NO: 11;
c. a first guide sequence that binds to a nucleotide sequence that is complementary to SEQ ID NO: 7 and a second guide sequence that binds to a nucleotide sequence that is complementary to SEQ ID NO: 12;
d. a first guide sequence that binds to a nucleotide sequence that is complementary to SEQ ID NO: 8 and a second guide sequence that binds to a nucleotide sequence that is complementary to SEQ ID NO: 10;
e. a first guide sequence that binds to a nucleotide sequence that is complementary to SEQ ID NO: 8 and a second guide sequence that binds to a nucleotide sequence that is complementary to SEQ ID NO: 11;
f. a first guide sequence that binds to a nucleotide sequence that is complementary to SEQ ID NO: 8 and a second guide sequence that binds to a nucleotide sequence that is complementary to SEQ ID NO: 12;
g. a first guide sequence that binds to a nucleotide sequence that is complementary to SEQ ID NO: 9 and a second guide sequence that binds to a nucleotide sequence that is complementary to SEQ ID NO: 10;
h. a first guide sequence that binds to a nucleotide sequence that is complementary to SEQ ID NO: 9 and a second guide sequence that binds to a nucleotide sequence that is complementary to SEQ ID NO: 11; or
i. a first guide sequence that binds to a nucleotide sequence that is complementary to SEQ ID NO: 9 and a second guide sequence that binds to a nucleotide sequence that is complementary to SEQ ID NO: 12.

In some embodiments, the RNA-targeted endonuclease is directed to a TSS and an E-box regulatory element of DMPK, and wherein a first pair of guide RNAs is administered, wherein the first pair of guide RNAs comprise at least 17, 18, 19, or 20 contiguous nucleotides of a first guide sequence selected from any one of SEQ ID NOs: 1, 2, and 3, and at least 17, 18, 19, or 20 contiguous nucleotides of a second guide sequence selected from any one of SEQ ID NOs: 4, 5, and 6; and a second pair of guide RNAs is administered, wherein the pair of guide RNAs comprise at least 17, 18, 19, or 20 contiguous nucleotides of a first guide sequence selected from any one of SEQ ID NOs: 7, 8, or 9, and at least 17, 18, 19, or 20 contiguous nucleotides of a second guide sequence selected from any one of SEQ ID NOs: 10, 11, or 12.

In some embodiments, the disclosure provides for a method of administering to a subject a composition comprising any one or more of the guides disclosed in Tables, 3A, 3B, 5A, 5B, 5C, 6A or 6B. In some embodiments, the composition comprises a guide sequence selected from any one of SEQ ID Nos: 1-11. In some embodiments, the composition comprises a guide sequence selected from any one of SEQ ID Nos: 1-11 and an endonuclease (e.g., SpCas9) or a nucleic acid encoding an endonuclease (e.g., SpCas9). In some embodiments, the composition comprises a guide sequence selected from any one of SEQ ID Nos: 1300-1308. In some embodiments, the composition comprises a guide sequence selected from any one of SEQ ID Nos: 1300-1308 and an endonuclease (e.g., SaCas9) or a nucleic acid encoding an endonuclease (e.g., SaCas9). In some embodiments, the composition comprises a guide sequence selected from any one of SEQ ID Nos: 1300-1308. In some embodiments, the composition comprises a guide sequence selected from any one of SEQ ID Nos: 1300-1308 and an endonuclease (e.g., SaCas9) or a nucleic acid encoding an endonuclease (e.g., SaCas9). In some embodiments, the composition comprises a guide sequence selected from any one of SEQ ID Nos: 1400-1496. In some embodiments, the composition comprises a guide sequence selected from any one of SEQ ID Nos: 1400-1496 and an endonuclease (e.g., SluCas9) or a nucleic acid encoding an endonuclease (e.g., SluCas9). In some embodiments, the composition comprises a guide sequence selected from any one of SEQ ID Nos: 1420, 1428, 1475, 1402, 1411, and 1463. In some embodiments, the composition comprises a guide sequence selected from any one of SEQ ID Nos: 1420, 1428, 1475, 1402, 1411, and 1463 and an endonuclease (e.g., SluCas9) or a nucleic acid encoding an endonuclease (e.g., SluCas9).

### IV. DNA-PK Inhibitor

In some embodiments, the method further comprises administrating a DNA-PK inhibitor (DNA-PKi). Where a DNA-PK inhibitor is used in a composition or method disclosed herein, it may be any DNA-PK inhibitor known in the art. DNA-PK inhibitors are discussed in detail, for example, in WO2014/159690; WO2013/163190; WO2018/013840; WO 2019/143675; WO 2019/143677; WO 2019/143678; US2014275059; US2013281431; US2020361877; US2020353101 and Robert et al., Genome Medicine (2015) 7:93, each of which are incorporated by reference herein. In some embodiments, the DNA-PK inhibitor is NU7441, KU-0060648, or any one of Compounds 1, 2, 3, 4, 5, or 6 (structures shown below), each of which is also described in at least one of the foregoing citations. In some embodiments, the DNA-PK inhibitor is Compound 1. In some embodiments, the DNA-PK inhibitor is Compound 2. In some embodiments, the DNA-PK inhibitor is Compound 6. In some embodiments, the DNA-PK inhibitor is Compound 3. Structures for exemplary DNA-PK inhibitors are as follows. Unless otherwise indicated, reference to a DNA-PK inhibitor by name or structure encompasses pharmaceutically acceptable salts thereof.

TABLE 2

| DNA-PK Inhibitor | Structure |
|---|---|
| NU7441 | 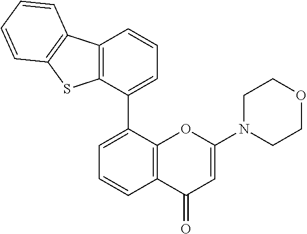 [SMILES: O=c1cc(N2CCOCC2)oc2c(-c3cccc4c3sc3ccccc34)cccc12] |
| KU-0060648 | 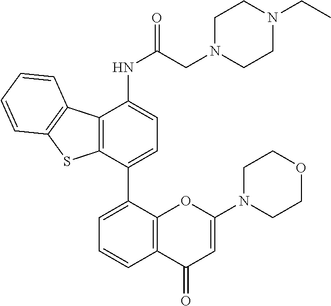 [SMILES: CCN1CCN(CC(=O)Nc2ccc(-c3cccc4c(=O)cc(N5CCOCC5)oc34)c3sc4ccccc4c23)CC1] |
| Compound 1 | 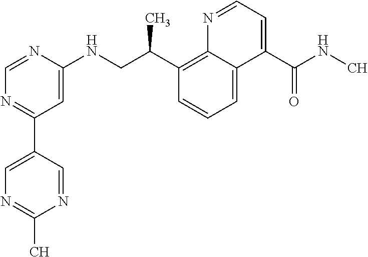 [SMILES: CNC(=O)c1ccnc2c([C@H](C)CNc3cc(-c4cnc(C)nc4)ncn3)cccc12] |
| Compound 2 | 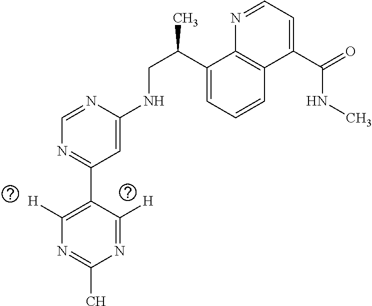 [SMILES: CNC(=O)c1ccnc2c([C@H](C)CNc3cc(-c4cnc(C)nc4)ncn3)cccc12] |
| Compound 3 | 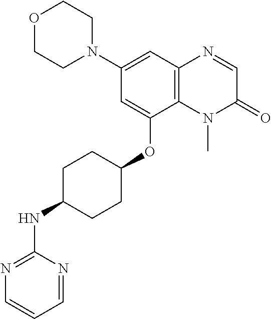 [SMILES: Cn1c(=O)cnc2cc(N3CCOCC3)cc(O[C@H]3CC[C@@H](Nc4ncccn4)CC3)c21] |
| Compound 4 | 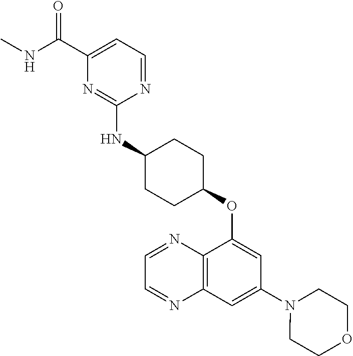 [SMILES: CNC(=O)c1ccnc(N[C@H]2CC[C@@H](Oc3cc(N4CCOCC4)cc4nccnc34)CC2)n1] |
| Compound 5 | 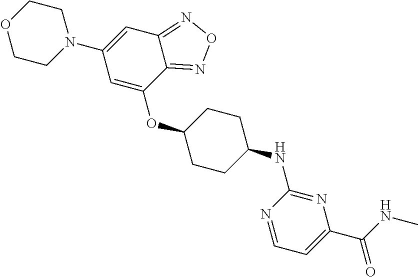 [SMILES: CNC(=O)c1ccnc(N[C@H]2CC[C@@H](Oc3cc(N4CCOCC4)cc4nonc34)CC2)n1] |
| Compound 6 | 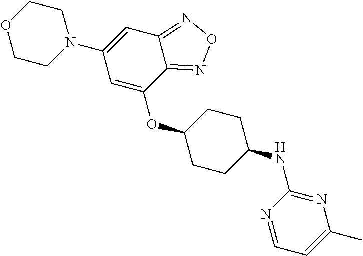 [SMILES: Cc1ccnc(N[C@H]2CC[C@@H](Oc3cc(N4CCOCC4)cc4nonc34)CC2)n1] |

In any of the foregoing embodiments where a DNA-PK inhibitor is used, it may be used in combination with only one gRNA or vector encoding only one gRNA to promote excision, i.e., the method does not always involve providing two or more guides that promote excision and/or disruption of one or more regulatory elements that at least partially drive expression of a gene, for example, in DM1, the CTG repeat.

In some embodiments where a DNA-PK inhibitor is used, it may be used in combination with a pair of gRNAs or vector encoding a pair of guide RNAs to promote excision. In some embodiments, the pair of gRNAs comprise gRNAs that are not the same. In particular embodiments, the pair of gRNAs together target one or more regulatory elements that at least partially drive expression of a gene that is known to produce toxic transcripts and/or proteins.

### V. Combination Therapy

In some embodiments, combination therapies comprising any of the methods or uses described herein together with an additional therapy suitable for ameliorating or treating, for example, DM1, DM2, Fuchs endothelial corneal dystrophy (FECD), Huntington disease-like 2 (HDL2), Huntington disease (HD), Spinocerebellar ataxia (SCA), Spinal and bulbar muscular atrophy (SBMA), Dentatorubral-pallidoluysian atrophy (DRPLA), Fragile X-associated tremor/ataxia syndrome (FXTAS), Frontotemporal dementia (FTD), amyotrophic lateral sclerosis (ALS), Oculopharyngeal muscular dystrophy (OPMD), Alzheimer's Disease (AD), Parkinson's Disease (PD) are provided. Suitable additional therapies for use in ameliorating various disorders characterized by toxic proteins and transcripts, such as those listed above, and/or one or more symptoms thereof are known in the art.

### VI. Delivery of Guide RNA Compositions

The methods and uses disclosed herein may use any suitable approach for delivering the guide RNAs and compositions described herein. Exemplary delivery approaches include vectors, such as viral vectors; lipid nanoparticles; transfection; and electroporation. In some embodiments, vectors or LNPs associated with the guide RNAs and nucleic acids encoding endonucleases disclosed herein are for use in preparing a medicament for treating DM1, DM2, Fuchs endothelial corneal dystrophy (FECD), Huntington disease-like 2 (HDL2), Huntington disease (HD), Spinocerebellar ataxia (SCA), Spinal and bulbar muscular atrophy (SBMA), Dentatorubral-pallidoluysian atrophy (DRPLA), Fragile X-associated tremor/ataxia syndrome (FXTAS), Frontotemporal dementia (FTD), amyotrophic lateral sclerosis (ALS), Oculopharyngeal muscular dystrophy (OPMD), Alzheimer's Disease (AD), Parkinson's Disease (PD).

Where a vector is used, it may be a viral vector, such as a non-integrating viral vector. In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), or AAV9 vector, wherein the number following AAV indicates the AAV serotype. Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors.

In some embodiments, the vector (e.g., viral vector, such as an adeno-associated viral vector) comprises a tissue-specific (e.g., muscle-specific) promoter, e.g., which is operatively linked to a sequence encoding the guide RNA. In some embodiments, the muscle-specific promoter is a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods Clin Dev. 2016; 3:16081, and references cited therein for detailed discussion of tissue-specific promoters including neuron-specific promoters. In some embodiments, a vector comprises a nucleic acid encoding one or more guide RNAs and a nucleic acid encoding a Cas protein (e.g., any of the Cas proteins disclosed herein). In some embodiments, the disclosure provides for a vector comprising a nucleic acid encoding one or more guide RNAs and a separate vector comprising a nucleic acid encoding a Cas protein. In some embodiments, the disclosure provides for a) a vector comprising a nucleic acid encoding one or more guide RNAs but not encoding a Cas protein, and b) a separate vector comprising a nucleic acid encoding a Cas protein and one or more additional guide RNAs.

In some embodiments, in addition to guide RNA and Cas protein sequences, the vectors further comprise nucleic acids that do not encode guide RNAs. Nucleic acids that do not encode guide RNA and Cas protein include, but are not limited to, promoters, enhancers, and regulatory sequences. In some embodiments, the vector comprises one or more nucleotide sequence(s) encoding a crRNA, a trRNA, or a crRNA and trRNA.

Lipid nanoparticles (LNPs) are a known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

Electroporation is a well-known means for delivery of cargo, and any electroporation methodology may be used for delivering the single vectors disclosed herein.

In some embodiments, methods or uses for delivering any one or more of the vectors disclosed herein to an ex vivo or in vivo cell is provided, wherein the guide RNA is encoded by a vector, associated with an LNP, or in aqueous solution. In some embodiments, the guide RNA/LNP or guide RNA is also associated with a Cas protein (e.g., Cas9) or sequence encoding the Cas protein (e.g., in the same vector, LNP, or solution).

TABLE 3A
Exemplary Guide RNA Sequences

| SEQ | | | |
|---|---|---|---|
| ID NO | Strand | Protospacer/Guide Sequence | |
| 1 | − | TAGCACTGAAGGGTTCTGAA | 20 nt |
| 101 | − | TAGCACTGAAGGGTTCTGA | 19 nt |
| 102 | − | TAGCACTGAAGGGTTCTG | 18 nt |
| 103 | − | TAGCACTGAAGGGTTCT | 17 nt |
| 2 | − | TGTGAGGGGTTAAGGCTGGG | 20 nt |
| 201 | − | TGTGAGGGGTTAAGGCTGG | 19 nt |
| 202 | − | TGTGAGGGGTTAAGGCTG | 18 nt |
| 203 | − | TGTGAGGGGTTAAGGCT | 17 nt |
| 3 | + | TCTCTGGCCACTTCTCTCTG | 20 nt |
| 301 | + | CTCTGGCCACTTCTCTCTG | 19 nt |
| 302 | + | TCTGGCCACTTCTCTCTG | 18 nt |
| 303 | + | CTGGCCACTTCTCTCTG | 17 nt |
| 4 | − | GACAGGCAGACATGCAGCCA | 20 nt |
| 401 | − | GACAGGCAGACATGCAGCC | 19 nt |
| 402 | − | GACAGGCAGACATGCAGC | 18 nt |
| 403 | − | GACAGGCAGACATGCAG | 17 nt |
| 5 | + | ATGTTGGACAGGCAGCACCA | 20 nt |
| 501 | + | TGTTGGACAGGCAGCACCA | 19 nt |
| 502 | + | GTTGGACAGGCAGCACCA | 18 nt |
| 503 | + | TTGGACAGGCAGCACCA | 17 nt |
| 6 | − | CCAACATGTCAGCCGAGGTG | 20 nt |
| 601 | − | CCAACATGTCAGCCGAGGT | 19 nt |
| 602 | − | CCAACATGTCAGCCGAGG | 18 nt |
| 603 | − | CCAACATGTCAGCCGAG | 17 nt |
| 7 | − | CATGGCTTAGGAGTCACAGC | 20 nt |
| 701 | − | CATGGCTTAGGAGTCACAG | 19 nt |
| 702 | − | CATGGCTTAGGAGTCACA | 18 nt |
| 703 | − | CATGGCTTAGGAGTCAC | 17 nt |
| 8 | + | TGACCCTGACCCTGACCCTG | 20 nt |
| 801 | + | GACCCTGACCCTGACCCTG | 19 nt |
| 802 | + | ACCCTGACCCTGACCCTG | 18 nt |
| 803 | + | CCCTGACCCTGACCCTG | 17 nt |
| 9 | + | CCCAGGCCACAGGAAACCAG | 20 nt |
| 901 | + | CCAGGCCACAGGAAACCAG | 19 nt |
| 902 | + | CAGGCCACAGGAAACCAG | 18 nt |
| 903 | + | AGGCCACAGGAAACCAG | 17 nt |
| 10 | + | TTGTCCCTCCTGGATCGCAG | 20 nt |
| 1001 | + | TGTCCCTCCTGGATCGCAG | 19 nt |
| 1002 | + | GTCCCTCCTGGATCGCAG | 18 nt |
| 1003 | + | TCCCTCCTGGATCGCAG | 17 nt |
| 11 | + | GGAATGGTGGCTGACCCACA | 20 nt |
| 1101 | + | GAATGGTGGCTGACCCACA | 19 nt |
| 1102 | + | AATGGTGGCTGACCCACA | 18 nt |
| 1103 | + | ATGGTGGCTGACCCACA | 17 nt |
| 12 | + | AACCAAACACCAGTCACTGA | 20 nt |
| 1201 | + | ACCAAACACCAGTCACTGA | 19 nt |
| 1202 | + | CCAAACACCAGTCACTGA | 18 nt |
| 1203 | + | CAAACACCAGTCACTGA | 17 nt |
| 1300 | − | ATGGCCTCTCTGCACCCCGCCT | 22 nt |
| 1301 | + | GACCCTGACCCTGACCCTGAGG | 22 nt |
| 1302 | − | TCTCTGCACCCCGCCTCAGGGT | 22 nt |
| 1303 | − | CACCCCGCCTCAGGGTCAGGGT | 22 nt |
| 1304 | + | CCATAGAGCCCACTTTTGGGGG | 22 nt |
| 1305 | + | CTGGTTCTCCCACAGGGCCCGC | 22 nt |
| 1306 | + | GAAGAACCCAAAGTTGTCCCTC | 22 nt |
| 1307 | - | CTGCGATCCAGGAGGGACAACT | 22 nt |
| 1308 | - | GGAGGGACAACTTTGGGTTCTT | 22 nt |
| 1400 | + | GTGGGTGCAGAACCTACAAAAG | 22 nt |
| 1401 | + | GAAGAACCCAAAGTTGTCCCTC | 22 nt |
| 1402 | + | AAGTTGTCCCTCCTGGATCGCA | 22 nt |
| 1403 | + | TTGTCCCTCCTGGATCGCAGAG | 22 nt |
| 1404 | + | TGTCCCTCCTGGATCGCAGAGG | 22 nt |
| 1405 | + | GTCCCTCCTGGATCGCAGAGGA | 22 nt |
| 1406 | + | TCCCTCCTGGATCGCAGAGGAG | 22 nt |
| 1407 | + | CTGGATCGCAGAGGAGGGGGCA | 22 nt |
| 1408 | + | GATCGCAGAGGAGGGGGCACTG | 22 nt |
| 1409 | + | CTGGTGGCGCCTGTCTGCAAAG | 22 nt |
| 1410 | + | GTCTGCAAAGCTGGTTCTCCCA | 22 nt |
| 1411 | + | TCTGCAAAGCTGGTTCTCCCAC | 22 nt |
| 1412 | + | TGAGTCACCGCCCTCCCAGTGC | 22 nt |
| 1413 | + | GAGTCACCGCCCTCCCAGTGCC | 22 nt |
| 1414 | + | CCTCCCAGTGCCTGGGCACCTG | 22 nt |
| 1415 | + | CTCCCAGTGCCTGGGCACCTGT | 22 nt |
| 1416 | + | TCCCAGTGCCTGGGCACCTGTT | 22 nt |
| 1417 | + | GCCTGGGCACCTGTTGGGGCAG | 22 nt |
| 1418 | + | GGCACCTGTTGGGGCAGCTGGA | 22 nt |
| 1419 | + | CCTGTTGGGGCAGCTGGAGAGG | 22 nt |
| 1420 | + | GCTGGCGCCGAACACCTGCCTG | 22 nt |
| 1421 | + | CACCTGCCTGTCGGCTGCGCCC | 22 nt |
| 1422 | + | CGCCCCTGGCAGCTGCCCCATG | 22 nt |
| 1423 | + | CAGCTGCCCCATGCTGGCCCTC | 22 nt |
| 1424 | + | TGCTGGCCCTCCTGGCTTGCCC | 22 nt |
| 1425 | + | CCTCCTGGCTTGCCCCAGGCCA | 22 nt |
| 1426 | + | CTTGCCCCAGGCCACAGGAAAC | 22 nt |
| 1427 | + | TTGCCCCAGGCCACAGGAAACC | 22 nt |
| 1428 | + | TGCCCCAGGCCACAGGAAACCA | 22 nt |
| 1429 | + | CAGGCCACAGGAAACCAGGGGA | 22 nt |
| 1430 | + | AGGCCACAGGAAACCAGGGGAG | 22 nt |
| 1431 | + | GAGAGGGCCATAGAGCCCACTT | 22 nt |
| 1432 | + | AGAGGGCCATAGAGCCCACTTT | 22 nt |
| 1433 | + | GAGGGCCATAGAGCCCACTTTT | 22 nt |
| 1434 | + | AGGGCCATAGAGCCCACTTTTG | 22 nt |
| 1435 | + | GGGCCATAGAGCCCACTTTTGG | 22 nt |
| 1436 | + | CCATAGAGCCCACTTTTGGGGG | 22 nt |
| 1437 | + | CATAGAGCCCACTTTTGGGGGG | 22 nt |
| 1438 | + | CCCACTTTTGGGGGGAGGGTCC | 22 nt |
| 1439 | + | TTTTGGGGGGAGGGTCCTAGGA | 22 nt |
| 1440 | + | TTTGGGGGGAGGGTCCTAGGAG | 22 nt |
| 1441 | + | GCATGACCCTGACCCTGACCCT | 22 nt |
| 1442 | + | TGACCCTGACCCTGACCCTGAG | 22 nt |
| 1443 | + | GACCCTGACCCTGACCCTGAGG | 22 nt |
| 1444 | + | ACCCTGACCCTGACCCTGAGGC | 22 nt |
| 1445 | + | TGACCCTGAGGCGGGGTGCAGA | 22 nt |
| 1446 | + | TGAGGCGGGGTGCAGAGAGGCC | 22 nt |
| 1447 | + | GAGGCGGGGTGCAGAGAGGCCA | 22 nt |
| 1448 | − | CTGGGTGTGTCTCCTTCTTTTG | 22 nt |
| 1449 | − | GAGGGACAACTTTGGGTTCTTC | 22 nt |
| 1450 | − | GGAGGGACAACTTTGGGTTCTT | 22 nt |
| 1451 | − | TGCGATCCAGGAGGGACAACTT | 22 nt |
| 1452 | − | CTGCGATCCAGGAGGGACAACT | 22 nt |
| 1453 | − | GCCCCCTCCTCTGCGATCCAGG | 22 nt |
| 1454 | − | TGCCCCCTCCTCTGCGATCCAG | 22 nt |
| 1455 | − | CAGTGCCCCCTCCTCTGCGATC | 22 nt |
| 1456 | − | GTGGGAGAACCAGCTTTGCAGA | 22 nt |
| 1457 | − | GCGGTGACTCACGCGGGCCCTG | 22 nt |
| 1458 | − | GGCGGTGACTCACGCGGGCCCT | 22 nt |
| 1459 | − | ACTGGGAGGGCGGTGACTCACG | 22 nt |
| 1460 | − | CACTGGGAGGGCGGTGACTCAC | 22 nt |
| 1461 | − | CAGGTGCCCAGGCACTGGGAGG | 22 nt |
| 1462 | − | CAACAGGTGCCCAGGCACTGGG | 22 nt |
| 1463 | − | CCAACAGGTGCCCAGGCACTGG | 22 nt |
| 1464 | − | GCCCCAACAGGTGCCCAGGCAC | 22 nt |
| 1465 | − | TGCCCCAACAGGTGCCCAGGCA | 22 nt |
| 1466 | − | TCCAGCTGCCCCAACAGGTGCC | 22 nt |
| 1467 | − | CCAGCCTCTCCAGCTGCCCCAA | 22 nt |
| 1468 | − | GGCGCAGCCGACAGGCAGGTGT | 22 nt |
| 1469 | − | TGCCAGGGGCGCAGCCGACAGG | 22 nt |
| 1470 | − | CAGCTGCCAGGGGCGCAGCCGA | 22 nt |
| 1471 | − | GGCCAGCATGGGGCAGCTGCCA | 22 nt |
| 1472 | − | GGGCCAGCATGGGGCAGCTGCC | 22 nt |
| 1473 | − | AGGGCCAGCATGGGGCAGCTGC | 22 nt |
| 1474 | − | GGCAAGCCAGGAGGGCCAGCAT | 22 nt |
| 1475 | − | GGGCAAGCCAGGAGGGCCAGCA | 22 nt |
| 1476 | − | GGGGCAAGCCAGGAGGGCCAGC | 22 nt |
| 1477 | − | CTGTGGCCTGGGGCAAGCCAGG | 22 nt |
| 1478 | − | CCTGTGGCCTGGGGCAAGCCAG | 22 nt |
| 1479 | − | TTTCCTGTGGCCTGGGGCAAGC | 22 nt |
| 1480 | − | CTCCCCTGGTTTCCTGTGGCCT | 22 nt |
| 1481 | − | TCTCCCCTGGTTTCCTGTGGCC | 22 nt |
| 1482 | − | CTCTCCCCTGGTTTCCTGTGGC | 22 nt |
| 1483 | − | TGGCCCTCTCCCCTGGTTTCCT | 22 nt |
| 1484 | − | GTGGGCTCTATGGCCCTCTCCC | 22 nt |
| 1485 | − | CCTCCCCCCAAAAGTGGGCTCT | 22 nt |
| 1486 | − | CCTAGGACCCTCCCCCCAAAAG | 22 nt |
| 1487 | − | TCCTAGGACCCTCCCCCCAAAA | 22 nt |
| 1488 | − | TCATGCTGGGAGCTCCCTCTCC | 22 nt |
| 1489 | − | AGGGTCAGGGTCAGGGTCATGC | 22 nt |
| 1490 | − | CAGGGTCAGGGTCAGGGTCATG | 22 nt |
| 1491 | − | ACCCCGCCTCAGGGTCAGGGTC | 22 nt |
| 1492 | − | CACCCCGCCTCAGGGTCAGGGT | 22 nt |
| 1493 | − | CTCTGCACCCCGCCTCAGGGTC | 22 nt |
| 1494 | − | TCTCTGCACCCCGCCTCAGGGT | 22 nt |
| 1495 | − | TGGCCTCTCTGCACCCCGCCTC | 22 nt |
| 1496 | − | ATGGCCTCTCTGCACCCCGCCT | 22 nt |

### EXAMPLES

The following examples are provided to illustrate certain disclosed embodiments and are not to be construed as limiting the scope of this disclosure in any way.

### VII. Example 1: In Vitro Study of SpCas9

### 1. Materials and Methods

A. SpCas9 sgRNA Selection.

sgRNAs targeting DMPK TSS and E-boxes were designed, and 12 exemplary sgRNA protospacer sequences (20-nucleotide in length) adjacent to the canonical NGG protospacer adjacent motif (PAM) and having >50% on-target efficiency scores were selected (Table 3B).

TABLE 3B
SpCas9 sgRNA

| SpCas9 | | | | Protospacer_ | Protospacer_ | |
|---|---|---|---|---|---|---|
| sgRNA | SEQ | | Protospacer sequence | start | end | PAM |
| name | ID NO | Strand | (20 nt) | (hg38) | (hg38) | sequence |
| TU1 | 1 | − | TAGCACTGAAGGGTTCTGAA | 45782619 | 45782638 | GGG |
| TU2 | 2 | − | TGTGAGGGGTTAAGGCTGGG | 45782565 | 45782584 | AGG |
| TU3 | 3 | + | TCTCTGGCCACTTCTCTCTG | 45782486 | 45782505 | CGG |
| TD1 | 4 | − | GACAGGCAGACATGCAGCCA | 45782447 | 45782466 | GGG |
| TD2 | 5 | + | ATGTTGGACAGGCAGCACCA | 45782351 | 45782370 | TGG |
| TD3 | 6 | − | CCAACATGTCAGCCGAGGTG | 45782338 | 45782357 | CGG |
| EU1 | 7 | − | CATGGCTTAGGAGTCACAGC | 45781935 | 45781954 | AGG |
| EU2 | 8 | + | TGACCCTGACCCTGACCCTG | 45781869 | 45781888 | AGG |
| EU3 | 9 | + | CCCAGGCCACAGGAAACCAG | 45781789 | 45781808 | GGG |
| ED1 | 10 | + | TTGTCCCTCCTGGATCGCAG | 45781594 | 45781613 | AGG |
| ED2 | 11 | + | GGAATGGTGGCTGACCCACA | 45781485 | 45781504 | CGG |
| ED3 | 12 | + | AACCAAACACCAGTCACTGA | 45781217 | 45781236 | GGG |

### B. Primary Myoblast Culture.

Primary healthy myoblasts (P01431-18F) and DM1 patient myoblasts (03001-32F) were obtained from Cook MyoSite. Myoblasts were maintained in myoblast growth medium consisting of Skeletal Muscle Cell Growth Basal Medium-2 (Lonza, CC-3246) and Skeletal Muscle Cell Growth Medium-2 SingleQuots Supplements and Growth Factors (Lonza, CC-3244) and purified with EasySep Human CD56 Positive Selection Kit II (StemCell Tech, 17855) following the manufacturer's instruction. Purified myoblasts were then banked in liquid nitrogen tank until 3-4 days before nucleofection.

### C. Preparation of RNPs.

RNPs were assembled with recombinant SpCas9 protein (Aldevron) and chemically modified sgRNAs (Synthego) at a ratio of 1:3 (protein: sgRNA). RNP complexes were first assembled for individual sgRNAs with 20 pmol of SpCas9 protein and 60 pmol of sgRNAs in 5 μL of P5 Nucleofector Solution. After incubation at room temperature for 20 minutes, the two RNP complexes (one for upstream sgRNA and one for downstream sgRNA) were mixed at 1:1 ratio and then further mixed with two hundred thousand primary myoblasts resuspended in 10 μL of P5 Nucleofector Solution.

D. Nucleofection of RNPs into Primary DM1 Myoblasts.

The Nucleofector 96-well Shuttle System (Lonza) was used to deliver the SpCa9/sgRNA RNPs into primary DM1 patient myoblasts using the nucleofection program CM138.

### E. Genomic DNA Extraction, PCR Amplification and Gel Electrophoresis.

Genomic DNA of DM1 myoblasts was isolated with the Kingfisher Flex purification system (Thermal Fisher) in 96-well format following the manufacturer's instruction. The TSS+E-box region was amplified using GoTaq Green Master Mix (Promega) and PCR primers flanking DMPK TSS and E-boxes. The forward primer sequence used was CATCACACCTGTGGCTTCCT (SEQ ID NO. 13), and the reverse primer sequence was TACCCTGCCAACCCAACTTC (SEQ ID NO. 14). Amplification was conducted using the following cycling parameters: 1 cycle at 95° C. for 2 min; 30 cycles of 95° C. for 30 sec, 64° C. for 30 sec, and 72° C. for 2 min; 1 cycle at 72° C. for 10 min. The PCR products were analyzed by gel electrophoresis.

F. mRNA Isolation and cDNA Preparation.

mRNA of DM1 myoblasts/myotubes was isolated with the Kingfisher Flex purification system (Thermal Fisher) in 96-well format following the manufacturer's instruction. cDNA was synthesized with SuperScript™ IV VILO™ Master Mix with ezDNase™ Enzyme (Thermal Fisher) following mRNA isolation.

G. ddPCR.

The primers and probes of ddPCR were designed using online primer design software. For mis-splicing assessment, three target primers/probe sets were used to detect exon inclusion (spliced-in) isoform of BIN1, KIF13a and DMD, respectively, and three reference primers/probe sets were used to detect the total expression level of BIN1, KIF13a and DMD, respectively. The ddPCR reference primers and probes used to detect the splicing profiles are described in Table 4. For DMPK expression, one target primer/probe set was used to detect DMPK expression levels, and one reference primer/probe set was used to detect GAPDH expression levels.

TABLE 4
ddPCR primer/probe sets for detecting gene′s alternative splicing

| ddPCR | Oligo | | | SEQ ID |
|---|---|---|---|---|
| set | type | Name | Sequence (5′ to 3′) | NO. |
| BIN1 | Fwd | cnTaqBIN1F1 | GAGCAACACCTTCACGGTCAA | 15 |
| | Primer | | | |
| | Rev | cnTaqBIN1R1 | CGCGTTGTCACTGTTCTTCTTT | 16 |
| | Primer | | | |
| | Probe | cnTaqBIN1Probe1 | TAAACTGTTTTCGCGGCTG | 17 |
| DMD | Fwd | cnTaqDMDF3 | TCCCTAGTTCAAGAGGAAGAAATACC | 18 |
| | Primer | | | |
| | Rev | cnTaqDMDR1 | TGCCATGTGGAAAAGACTTCCTA | 19 |
| | Primer | | | |
| | Probe | cnTaqDMDProbe3 | CTGGAAAGCCAATGAG | 20 |
| KIF13A | Fwd | cnTaqKIF13AF | TGATGAGGATGGTGATGATATGG | 21 |
| | Primer | | | |
| | Rev | cnTaqKIF13AR | ATCTGACCACCTCTCCCTTACG | 22 |
| | Primer | | | |
| | Probe | cnTaqKIF13AProbe | TAGTTATCAGGAAGAAGACTTA | 23 |

Primers are indicated as forward or reverse primers using Fwd and Rev, respectively.

The 24 μL of ddPCR reaction consisted of 12 μL of Supermix for Probes (no dUTP) (Bio-Rad Laboratories), 1 μL of Reference primers mix (21.6 μM), 1 μL of Reference probe (6 μM), 1 μL of Target primers mix (21.6 μM), 1 μL of Target probe (6 μM), 2 μL of H₂O and 6 μL of sample genomic DNA. Droplets were generated using probe oil with the QX200 Droplet Generator (Bio-Rad Laboratories). Droplets were transferred to a 96-well PCR plate, sealed and cycled in a C1000 deep well Thermocycler (Bio-Rad Laboratories) under the following cycling protocol: 1 cycle at 95° C. for 10 min; 40 cycles of 94° C. for 30 sec, and 57° C. for 1 min; 1 cycle at 98° C. for 10 min (for enzyme inactivation). The cycled plate was then transferred and read in the FAM and HEX channels using the Bio-Rad QX200 Droplet Reader (Bio-Rad Laboratories). ddPCR analysis was performed with the Bio-Rad QuantaSoft Pro Software.

### H. FISH Staining of RNA Foci.

Primary myoblasts were fixed for 15 min with 4% paraformaldehyde (PFA) and washed five times with 1×PBS for 10 min each at room temperature. Before staining, cells were permeabilized with 0.5% triton X-100 in 1×PBS for 5 min at room temperature, and then washed with 30% formamide and 2× saline-sodium citrate (SSC) mixture for 10 min at room temperature. Cells were then stained with 1 ng/μL of Cy3-PNA(CAG)₅ probe (PNA Bio, F5001) diluted in 30% formamide, 2×SSC, 2 μg/mL BSA, 66 μg/mL yeast tRNA, and 2 mM vanadyl complex for 15 min at 80° C. Following probe staining, cells were then washed in 30% formamide and 2×SSC mixture for 30 min at 42° C., then washed in 30% formamide and 2×SSC mixture for 30 min at 37° C., and then washed in 1×SSC solution for 10 min at room temperature, and finally washed in 1×PBS for 10 min at room temperature. For staining of myotube nuclei, cells were stained with anti-Myogenin antibody diluted in 1% bovine serum albumin (BSA) for overnight at 4° C. and washed twice with 1×PBS for 10 min each at room temperature. Cells were then incubated with the secondary antibody goat anti-rabbit Alexa 647 (Thermo Fisher, A32728) diluted in 1% BSA for 1 hr at room temperature and washed twice with 1×PBS for 10 min each at room temperature. Next, cells were stained with Hoechst solution (Thermo Fisher, H3569) at 0.1 mg/ml for 5 min and washed once with 1×PBS for 5 min. PBS was aspirated and fresh 100 μl of fresh PBS was added to each well. High-throughput acquisition of images was completed with the ImageXpress Micro Confocal High-Content Imaging System (Molecular Devices). RNA foci quantifications were accomplished with a customized analysis module of the MetaXpress program (Molecular Devices).

### 2. In Vitro Results

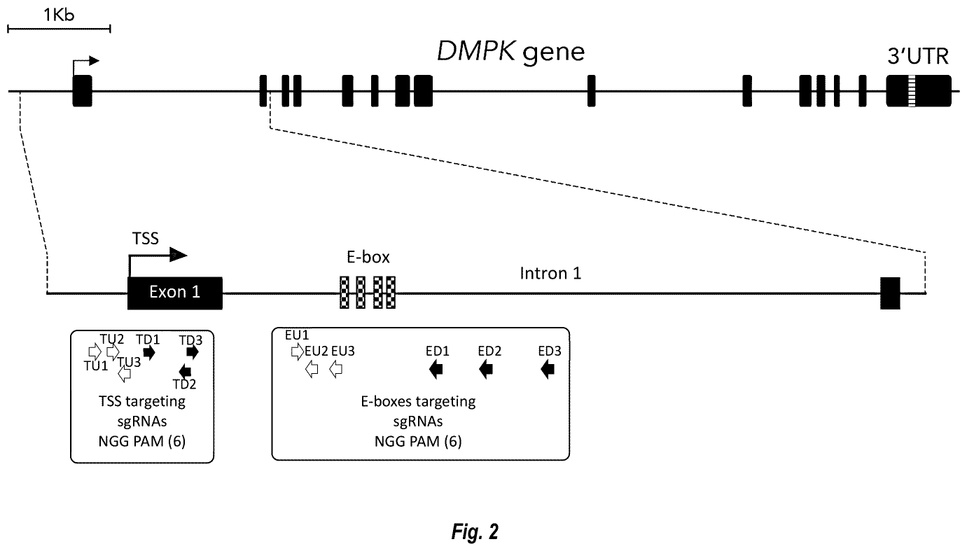

Six NGG Protospacer Adjacent Motif (PAM) SpCas9 sgRNAs were selected for targeting the TSS of the human DMPK gene: three sgRNAs (TU1-TU3) are located upstream of the DMPK TSS and three (TD1-TD3) are located downstream of the DMPK TSS (Table 3B and FIG. 2). Similarly, six SpCas9 sgRNAs were selected to target the four E-box elements within intron 1 of human DMPK gene: three sgRNAs (EU1-EU3) are located upstream of the four E-box elements and three (ED1-ED3) are located downstream of the four E-box elements (Table 3B and FIG. 2).

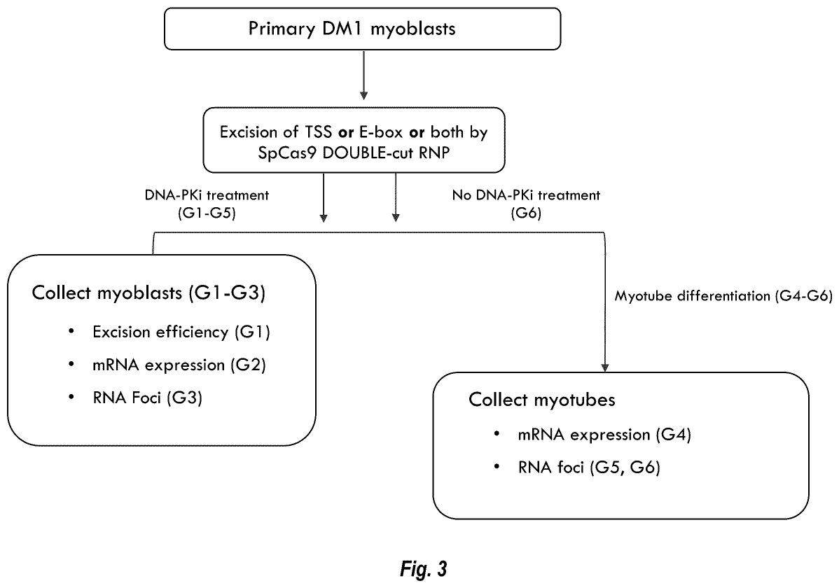

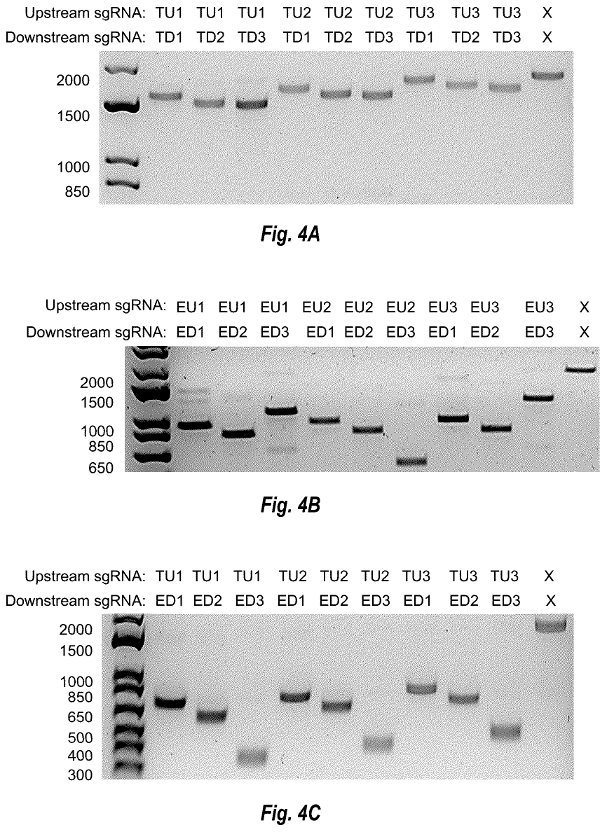

For the study, the 12 upstream and downstream SpCas9 sgRNAs described above were paired, which resulted in 27 sgRNA pairs. The first nine pairs (three TU sgRNAs paired with three TD sgRNAs) targeted the TSS of DMPK, the second nine pairs (three EU sgRNAs paired with three ED sgRNAs) targeted the E-box region; and the third nine pairs (three TU sgRNAs paired with three ED sgRNAs) targeted both the TSS and the E-box region (FIGS. 4-7). Each SpCas9 sgRNA pair consisting of one upstream sgRNA and one downstream sgRNA were assembled with recombinant SpCas9 protein to form a ribonucleoprotein (RNP) complex and delivered to primary DM1 patient myoblasts using the Amaxa 4D-Nucleofector. Post nucleofection, myoblasts were divided into six groups, and were treated with (Groups 1-5) or without (Group 6) 3 μM DNA-dependent Protein Kinase Inhibitor (DNA-PKi) Compound 6 for 24 hrs. DNA-PKi was used to enhance excision efficiency, in an effort to more accurately ascertain the contribution of each regulatory region to DMPK transcription. 72 hrs post nucleofection, myoblasts from Groups 1-3 were subjected to genomic DNA isolation, mRNA isolation, and RNA foci staining, whereas Groups 4-6 were subjected to myogenic differentiation to form myotubes. Myotubes were collected at day 15 post nucleofection (Day 12 post differentiation) for mRNA isolation (Group 4) and RNA foci staining (Groups 5, 6) (FIG. 3).

First, excision of TSS and/or E-boxes was evaluated by PCR using genomic DNA prepared from Group 1 myoblast samples and a primer set flanking the TSS and E-box region. Downshifted PCR bands were observed from all paired sgRNAs-nucleofected samples compare to DM1 mock control, indicating successful excision events (FIGS. 4A-4C).

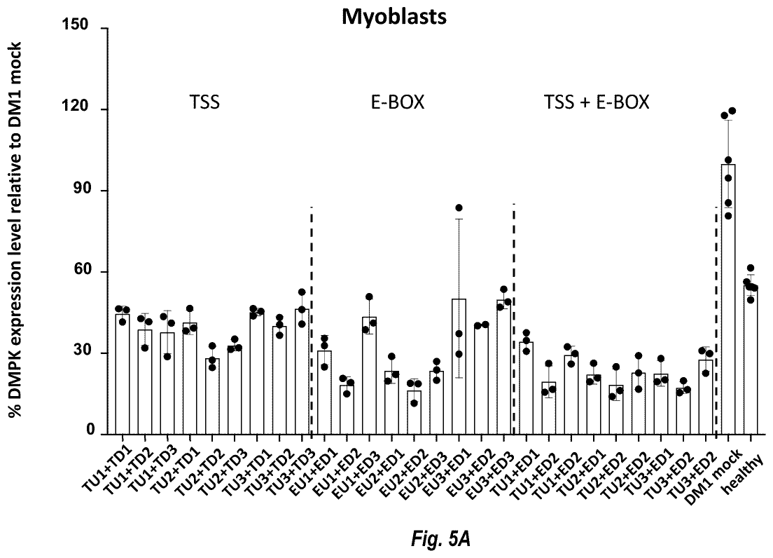

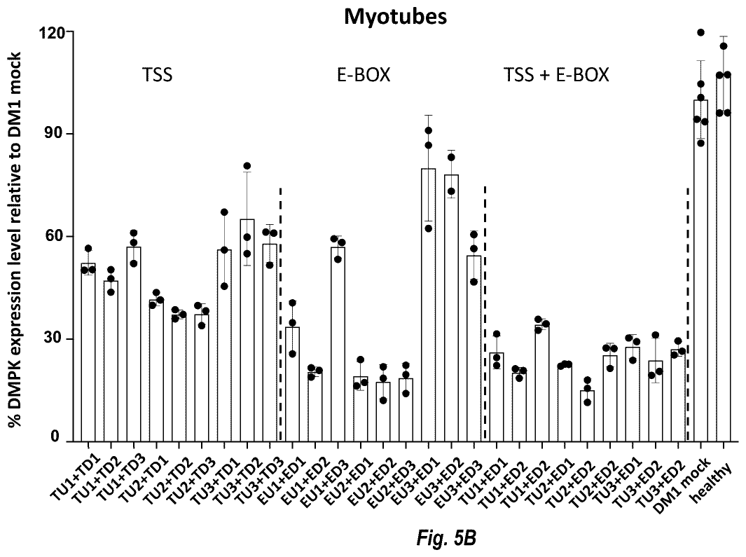

Next, DMPK mRNA expression levels in DM1 myoblasts (Group 2) and myotubes (Group 4) were assessed using ddPCR. Of all the sgRNA pairs tested, four targeting the E-box region, and eight targeting both TSS and E-box elements (TSS+E-Box) showed at least a 70% reduction of DMPK transcript levels in both myoblasts (FIG. 5A) and myotubes (FIG. 5B) as compared to control, indicating a synergistic effect of E-boxes and TSS in regulating DMPK expression in myogenic cells.

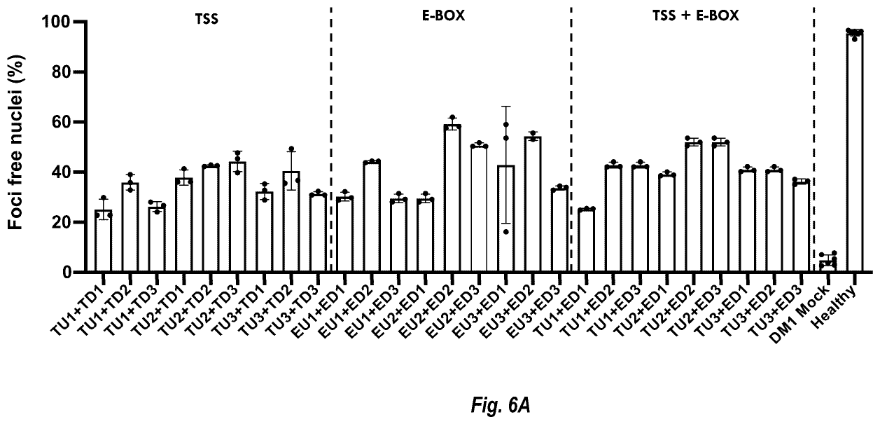

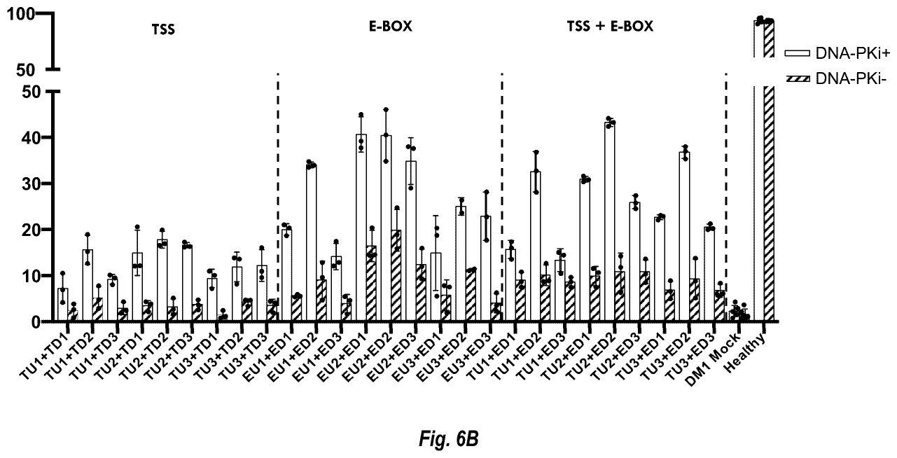

FISH staining of RNA foci showed robust reduction of CUG foci in primary DM1 patient myoblasts with DNA-PKi treatment (Group 3). Eleven sgRNA pairs (two pairs targeting TSS, five pairs targeting E-boxes, four pairs targeting both TSS and E-boxes) abolished CUG RNA foci in at least 40% myoblast nuclei (FIG. 6A), which might significantly alleviate DM1 phenotypes. DNA-PKi-treated DM1 patient myotubes (Group 5) showed substantially higher foci-free myonuclei than vehicle-treated DM1 myotubes (Group 6; FIG. 6B).

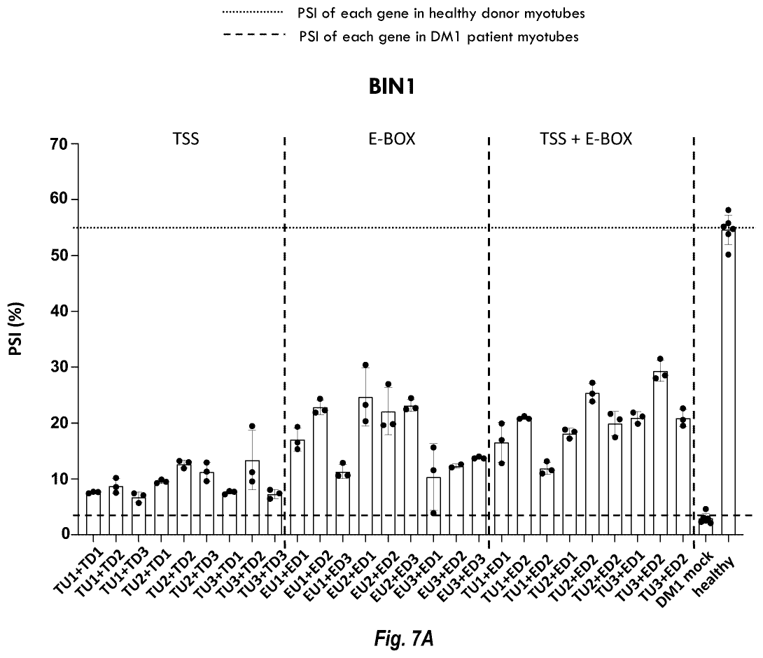

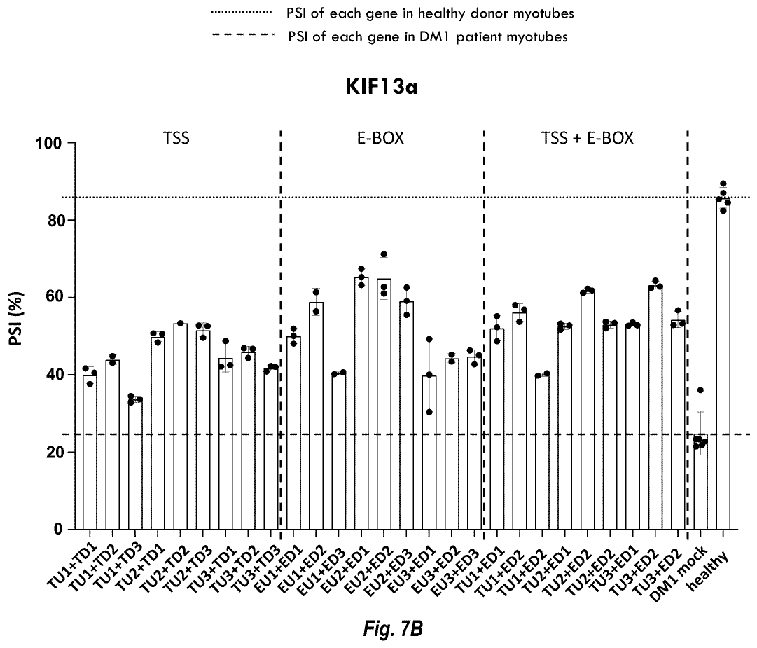

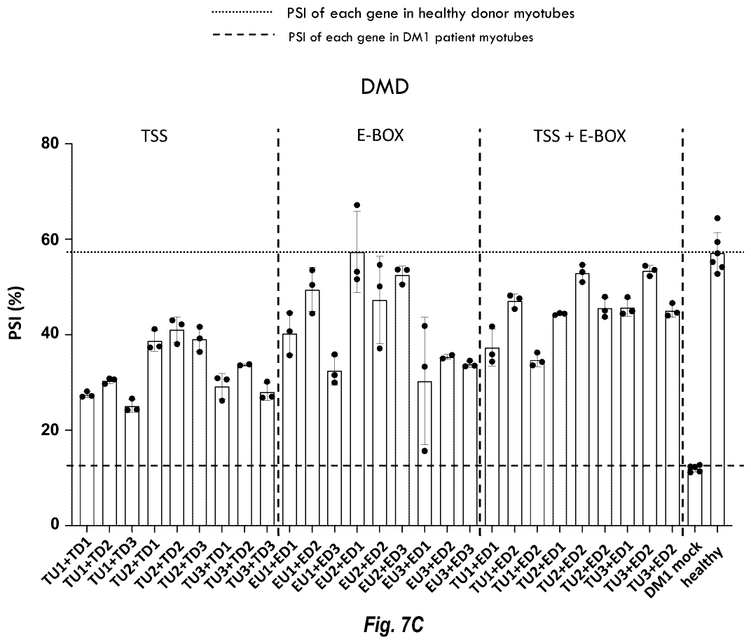

Finally, the alternative splicing profile of three genes (BIN1, KIF13a and DMD) in DM1 myotubes treated with paired sgRNAs (Group 4) was analyzed. Mis-splicing defects in these genes were improved by all 27 pairs of sgRNAs analyzed. In particular, sgRNA pairs targeting E-boxes or TSS+E-boxes showed the most robust effect (FIG. 7).

### VIII. Example 2: In Vitro Study of SaCas9 and SluCas9

### 1. Materials and Methods

A. SaCas9 and SluCas9 sgRNA Selection.

sgRNAs targeting DMPK E-boxes were designed to include all possible guides within ±150 bp of E-box. This results in total of 9 saCas9 sgRNAs (Table 5A) and 97 sluCas9 sgRNAs (Table 5B). Nine SaCas9 sgRNA protospacer sequences (22-nucleotide in length) adjacent to the canonical NNGRRT PAM and 6 SluCas9 sgRNAs (Table 5C) with the NNGG PAM sequences were selected.

TABLE 5A
SaCas9 soRNA

| SaCas9 | | | | Protospacer_ | Protospacer_ | |
|---|---|---|---|---|---|---|
| sgRNA | SEQ | | Protospacer sequence | start | end | PAM |
| name | ID NO | Strand | (20 nt) | (hg38) | (hg38) | sequence |
| SaU1 | 1300 | − | ATGGCCTCTCTGCACCCCGCCT | 45781888 | 45781910 | CAGGGT |
| SaU2 | 1301 | + | GACCCTGACCCTGACCCTGAGG | 45781869 | 45781891 | CGGGGT |
| SaU3 | 1302 | − | TCTCTGCACCCCGCCTCAGGGT | 45781882 | 45781904 | CAGGGT |
| SaU4 | 1303 | − | CACCCCGCCTCAGGGTCAGGGT | 45781876 | 45781898 | CAGGGT |
| SaU5 | 1304 | + | CCATAGAGCCCACTTTTGGGGG | 45781817 | 45781839 | GAGGGT |
| SaD1 | 1305 | + | CTGGTTCTCCCACAGGGCCCGC | 45781646 | 45781668 | GTGAGT |
| SaD2 | 1306 | + | GAAGAACCCAAAGTTGTCCCTC | 45781580 | 45781602 | CTGGAT |
| SaD3 | 1307 | − | CTGCGATCCAGGAGGGACAACT | 45781591 | 45781613 | TTGGGT |
| SaD4 | 1308 | − | GGAGGGACAACTTTGGGTTCTT | 45781581 | 45781603 | CTGGGT |

TABLE 5B
SluCas9 sgRNA

| SaCas9 | | | | Protospacer_ | Protospacer_ | |
|---|---|---|---|---|---|---|
| sgRNA | SEQ | | Protospacer sequence | start | end | PAM |
| name | ID NO | Strand | (20 nt) | (hg38) | (hg38) | sequence |
| slu1 | 1400 | + | GTGGGTGCAGAACCTACAAAAG | 45781543 | 45781565 | AAGG |
| slu2 | 1401 | + | GAAGAACCCAAAGTTGTCCCTC | 45781580 | 45781602 | CTGG |
| slu3 | 1402 | + | AAGTTGTCCCTCCTGGATCGCA | 45781590 | 45781612 | GAGG |
| slu4 | 1403 | + | TTGTCCCTCCTGGATCGCAGAG | 45781593 | 45781615 | GAGG |
| slu5 | 1404 | + | TGTCCCTCCTGGATCGCAGAGG | 45781594 | 45781616 | AGGG |
| slu6 | 1405 | + | GTCCCTCCTGGATCGCAGAGGA | 45781595 | 45781617 | GGGG |
| slu7 | 1406 | + | TCCCTCCTGGATCGCAGAGGAG | 45781596 | 45781618 | GGGG |
| slu8 | 1407 | + | CTGGATCGCAGAGGAGGGGGCA | 45781602 | 45781624 | CTGG |
| slu9 | 1408 | + | GATCGCAGAGGAGGGGGCACTG | 45781605 | 45781627 | GTGG |
| slu10 | 1409 | + | CTGGTGGCGCCTGTCTGCAAAG | 45781624 | 45781646 | CTGG |
| slu11 | 1410 | + | GTCTGCAAAGCTGGTTCTCCCA | 45781636 | 45781658 | CAGG |
| slu12 | 1411 | + | TCTGCAAAGCTGGTTCTCCCAC | 45781637 | 45781659 | AGGG |
| slu13 | 1412 | + | TGAGTCACCGCCCTCCCAGTGC | 45781669 | 45781691 | CTGG |
| slu14 | 1413 | + | GAGTCACCGCCCTCCCAGTGCC | 45781670 | 45781692 | TGGG |
| slu15 | 1414 | + | CCTCCCAGTGCCTGGGCACCTG | 45781680 | 45781702 | TTGG |
| slu16 | 1415 | + | CTCCCAGTGCCTGGGCACCTGT | 45781681 | 45781703 | TGGG |
| slu17 | 1416 | + | TCCCAGTGCCTGGGCACCTGTT | 45781682 | 45781704 | GGGG |
| slu18 | 1417 | 1 | GCCTGGGCACCTGTTGGGGCAG | 45781689 | 45781711 | CTGG |
| slu19 | 1418 | + | GGCACCTGTTGGGGCAGCTGGA | 45781694 | 45781716 | GAGG |
| slu20 | 1419 | + | CCTGTTGGGGCAGCTGGAGAGG | 45781698 | 45781720 | CTGG |
| slu21 | 1420 | + | GCTGGCGCCGAACACCTGCCTG | 45781719 | 45781741 | TCGG |
| slu22 | 1421 | + | CACCTGCCTGTCGGCTGCGCCC | 45781731 | 45781753 | CTGG |
| slu23 | 1422 | + | CGCCCCTGGCAGCTGCCCCATG | 45781748 | 45781770 | CTGG |
| slu24 | 1423 | + | CAGCTGCCCCATGCTGGCCCTC | 45781757 | 45781779 | CTGG |
| slu25 | 1424 | + | TGCTGGCCCTCCTGGCTTGCCC | 45781768 | 45781790 | CAGG |
| slu26 | 1425 | + | CCTCCTGGCTTGCCCCAGGCCA | 45781775 | 45781797 | CAGG |
| slu27 | 1426 | + | CTTGCCCCAGGCCACAGGAAAC | 45781783 | 45781805 | CAGG |
| slu28 | 1427 | + | TTGCCCCAGGCCACAGGAAACC | 45781784 | 45781806 | AGGG |
| slu29 | 1428 | + | TGCCCCAGGCCACAGGAAACCA | 45781785 | 45781807 | GGGG |
| slu30 | 1429 | 1 | CAGGCCACAGGAAACCAGGGGA | 45781790 | 45781812 | GAGG |
| slu31 | 1430 | + | AGGCCACAGGAAACCAGGGGAG | 45781791 | 45781813 | AGGG |
| slu32 | 1431 | + | GAGAGGGCCATAGAGCCCACTT | 45781810 | 45781832 | TTGG |
| slu33 | 1432 | + | AGAGGGCCATAGAGCCCACTTT | 45781811 | 45781833 | TGGG |
| slu34 | 1433 | + | GAGGGCCATAGAGCCCACTTTT | 45781812 | 45781834 | GGGG |
| slu35 | 1434 | + | AGGGCCATAGAGCCCACTTTTG | 45781813 | 45781835 | GGGG |
| slu36 | 1435 | + | GGGCCATAGAGCCCACTTTTGG | 45781814 | 45781836 | GGGG |
| slu37 | 1436 | + | CCATAGAGCCCACTTTTGGGGG | 45781817 | 45781839 | GAGG |
| slu38 | 1437 | + | CATAGAGCCCACTTTTGGGGGG | 45781818 | 45781840 | AGGG |
| slu39 | 1438 | + | CCCACTTTTGGGGGGAGGGTCC | 45781825 | 45781847 | TAGG |
| slu40 | 1439 | + | TTTTGGGGGGAGGGTCCTAGGA | 45781830 | 45781852 | GAGG |
| slu41 | 1440 | + | TTTGGGGGGAGGGTCCTAGGAG | 45781831 | 45781853 | AGGG |
| slu42 | 1441 | + | GCATGACCCTGACCCTGACCCT | 45781865 | 45781887 | GAGG |
| slu43 | 1442 | + | TGACCCTGACCCTGACCCTGAG | 45781868 | 45781890 | GCGG |
| slu44 | 1443 | + | GACCCTGACCCTGACCCTGAGG | 45781869 | 45781891 | CGGG |
| slu45 | 1444 | + | ACCCTGACCCTGACCCTGAGGC | 45781870 | 45781892 | GGGG |
| slu46 | 1445 | + | TGACCCTGAGGCGGGGTGCAGA | 45781880 | 45781902 | GAGG |
| slu47 | 1446 | + | TGAGGCGGGGTGCAGAGAGGCC | 45781886 | 45781908 | ATGG |
| slu48 | 1447 | + | GAGGCGGGGTGCAGAGAGGCCA | 45781887 | 45781909 | TGGG |
| slu49 | 1448 | − | CTGGGTGTGTCTCCTTCTTTTG | 45781559 | 45781581 | TAGG |
| slu50 | 1449 | − | GAGGGACAACTTTGGGTTCTTC | 45781580 | 45781602 | TGGG |
| slu51 | 1450 | − | GGAGGGACAACTTTGGGTTCTT | 45781581 | 45781603 | CTGG |
| slu52 | 1451 | − | TGCGATCCAGGAGGGACAACTT | 45781590 | 45781612 | TGGG |
| slu53 | 1452 | − | CTGCGATCCAGGAGGGACAACT | 45781591 | 45781613 | TTGG |
| slu54 | 1453 | − | GCCCCCTCCTCTGCGATCCAGG | 45781601 | 45781623 | AGGG |
| slu55 | 1454 | − | TGCCCCCTCCTCTGCGATCCAG | 45781602 | 45781624 | GAGG |
| slu56 | 1455 | − | CAGTGCCCCCTCCTCTGCGATC | 45781605 | 45781627 | CAGG |
| slu57 | 1456 | − | GTGGGAGAACCAGCTTTGCAGA | 45781637 | 45781659 | CAGG |
| slu58 | 1457 | − | GCGGTGACTCACGCGGGCCCTG | 45781658 | 45781680 | TGGG |
| slu59 | 1458 | − | GGCGGTGACTCACGCGGGCCCT | 45781659 | 45781681 | GTGG |
| slu60 | 1459 | − | ACTGGGAGGGCGGTGACTCACG | 45781667 | 45781689 | CGGG |
| slu6 | 1460 | − | CACTGGGAGGGCGGTGACTCAC | 45781668 | 45781690 | GCGG |
| slu62 | 1461 | − | CAGGTGCCCAGGCACTGGGAGG | 45781680 | 45781702 | GCGG |
| slu63 | 1462 | − | CAACAGGTGCCCAGGCACTGGG | 45781683 | 45781705 | AGGG |
| slu64 | 1463 | − | CCAACAGGTGCCCAGGCACTGG | 45781684 | 45781706 | GAGG |
| slu65 | 1464 | − | GCCCCAACAGGTGCCCAGGCAC | 45781687 | 45781709 | TGGG |
| slu66 | 1465 | − | TGCCCCAACAGGTGCCCAGGCA | 45781688 | 45781710 | CTGG |
| slu67 | 1466 | − | TCCAGCTGCCCCAACAGGTGCC | 45781694 | 45781716 | CAGG |
| slu68 | 1467 | − | CCAGCCTCTCCAGCTGCCCCAA | 45781702 | 45781724 | CAGG |
| slu69 | 1468 | − | GGCGCAGCCGACAGGCAGGTGT | 45781730 | 45781752 | TCGG |
| slu70 | 1469 | − | TGCCAGGGGCGCAGCCGACAGG | 45781737 | 45781759 | CAGG |
| slu71 | 1470 | − | CAGCTGCCAGGGGCGCAGCCGA | 45781741 | 45781763 | CAGG |
| slu72 | 1471 | − | GGCCAGCATGGGGCAGCTGCCA | 45781754 | 45781776 | GGGG |
| slu73 | 1472 | − | GGGCCAGCATGGGGCAGCTGCC | 45781755 | 45781777 | AGGG |
| slu74 | 1473 | − | AGGGCCAGCATGGGGCAGCTGC | 45781756 | 45781778 | CAGG |
| slu75 | 1474 | − | GGCAAGCCAGGAGGGCCAGCAT | 45781767 | 45781789 | GGGG |
| slu76 | 1475 | − | GGGCAAGCCAGGAGGGCCAGCA | 45781768 | 45781790 | TGGG |
| slu77 | 1476 | − | GGGGCAAGCCAGGAGGGCCAGC | 45781769 | 45781791 | ATGG |
| slu78 | 1477 | − | CTGTGGCCTGGGGCAAGCCAGG | 45781778 | 45781800 | AGGG |
| slu79 | 1478 | − | CCTGTGGCCTGGGGCAAGCCAG | 45781779 | 45781801 | GAGG |
| slu80 | 1479 | − | TTTCCTGTGGCCTGGGGCAAGC | 45781782 | 45781804 | CAGG |
| slu81 | 1480 | − | CTCCCCTGGTTTCCTGTGGCCT | 45781791 | 45781813 | GGGG |
| slu82 | 1481 | − | TCTCCCCTGGTTTCCTGTGGCC | 45781792 | 45781814 | TGGG |
| slu83 | 1482 | − | CTCTCCCCTGGTTTCCTGTGGC | 45781793 | 45781815 | CTGG |
| slu84 | 1483 | − | TGGCCCTCTCCCCTGGTTTCCT | 45781798 | 45781820 | GTGG |
| slu85 | 1484 | − | GTGGGCTCTATGGCCCTCTCCC | 45781808 | 45781830 | CTGG |
| slu86 | 1485 | − | CCTCCCCCCAAAAGTGGGCTCT | 45781821 | 45781843 | ATGG |
| slu87 | 1486 | − | CCTAGGACCCTCCCCCCAAAAG | 45781829 | 45781851 | TGGG |
| slu88 | 1487 | − | TCCTAGGACCCTCCCCCCAAAA | 45781830 | 45781852 | GTGG |
| slu89 | 1488 | − | TCATGCTGGGAGCTCCCTCTCC | 45781849 | 45781871 | TAGG |
| slu90 | 1489 | − | AGGGTCAGGGTCAGGGTCATGC | 45781865 | 45781887 | TGGG |
| slu91 | 1490 | − | CAGGGTCAGGGTCAGGGTCATG | 45781866 | 45781888 | CTGG |
| slu92 | 1491 | − | ACCCCGCCTCAGGGTCAGGGTC | 45781875 | 45781897 | AGGG |
| slu93 | 1492 | − | CACCCCGCCTCAGGGTCAGGGT | 45781876 | 45781898 | CAGG |
| slu94 | 1493 | − | CTCTGCACCCCGCCTCAGGGTC | 45781881 | 45781903 | AGGG |
| slu95 | 1494 | − | TCTCTGCACCCCGCCTCAGGGT | 45781882 | 45781904 | CAGG |
| slu96 | 1495 | − | TGGCCTCTCTGCACCCCGCCTC | 45781887 | 45781909 | AGGG |
| slu97 | 1496 | − | ATGGCCTCTCTGCACCCCGCCT | 45781888 | 45781910 | CAGG |

TABLE 5C
Paired Slu Cas9 soRNA

| SaCas9 | | | | Protospacer_ | Protospacer_ | |
|---|---|---|---|---|---|---|
| sgRNA | SEQ | | Protospacer sequence | start | end | PAM |
| name | ID NO | Strand | (20 nt) | (hg38) | (hg38) | sequence |
| SluU1 | 1428 | + | TGCCCCAGGCCACAGGAAACCA | 45781785 | 45781807 | GGGG |
| SluU2 | 1475 | − | GGGCAAGCCAGGAGGGCCAGCA | 45781768 | 45781790 | TGGG |
| SluU3 | 1420 | + | GCTGGCGCCGAACACCTGCCTG | 45781719 | 45781741 | TCGG |
| SluD1 | 1463 | − | CCAACAGGTGCCCAGGCACTGG | 45781684 | 45781706 | GAGG |
| SluD2 | 1411 | + | TCTGCAAAGCTGGTTCTCCCAC | 45781637 | 45781659 | AGGG |
| SluD3 | 1402 | + | AAGTTGTCCCTCCTGGATCGCA | 45781590 | 45781612 | GAGG |
| SluU64- | 1497 | − | ggggcTCGAAGGGTCCTTGTAG | 45770273 | 45770294 | CGGG |
| CTG | | | | | | |
| SluD34- | 1498 | + | CGCGCCAGACGCTCCCCAGAGC | 45770014 | 45770035 | AGGG |
| CTG | | | | | | |

### B. Primary Myoblast Culture.

Primary healthy myoblasts (P01431-18F) and DM1 patient myoblasts (03001-32F) were obtained from Cook MyoSite. Myoblasts were maintained in myoblast growth medium consisted of Myotonic™ Basal Medium (Cook Myosite, MB-2222) and Myotonic™ Growth Supplement (Cook Myosite, MS-3333) and purified with EasySep Human CD56 Positive Selection Kit II (StemCell Tech, 17855) following the manufacturer's instruction. Purified myoblasts were then banked in liquid nitrogen tank until 3-4 days before nucleofection.

### C. Preparation of RNPs.

The RNPs were assembled with either recombinant SaCas9 protein (Aldevron) or SluCas9 (Aldevron) and chemically modified sgRNAs (Synthego) at a ratio of 1:3 (protein:sgRNA). Specifically, SaCas9 RNPs complexes were first assembled for individual sgRNAs with 20 pmol of SaCas9 protein and 60 pmol of sgRNAs in 5 μL of P5 Nucleofector Solution. SluCas9 RNPs complexes were first assembled for individual sgRNAs with 15 pmol of SluCas9 and 45 pmol of sgRNAs in 5 μL of P5 Nucleofector Solution.

After incubation at room temperature for 20 minutes, two RNP complexes, one for upstream sgRNA and one for downstream sgRNA, were mixed at 1:1 ratio and then further mixed with two hundred thousand of primary myoblasts resuspended in 10 μL of P5 Nucleofector Solution.

D. Nucleofection of RNPs into Primary DM1 Myoblasts.

The Nucleofector 96-well Shuttle System (Lonza) was used to deliver the SaCa9/sgRNA and SluCas9/sgRNA RNPs into primary DM1 patient myoblasts using the nucleofection program CM138.

### E. Genomic DNA Extraction, PCR Amplification and Gel Electrophoresis.

Genomic DNA of DM1 myoblasts was isolated with the Kingfisher Flex purification system (Thermal Fisher) in 96-well format following the manufacturer's instruction. The E-boxes region was amplified using Q5 High-Fidelity 2× Master Mix (New England biolabs) and PCR primers flanking DMPK E-boxes. The forward primer sequence is tcctatgccaccactctgg (SEQ ID NO: 27), and the reverse primer sequence is AACTAAAGGACGCAGGGAC (SEQ ID NO: 26). Amplification was conducted using the following cycling parameters: 1 cycle at 98° C. for 2 min; 30 cycles of 98° C. for 30 sec, 66° C. for 30 sec, and 72° C. for 2 min; 1 cycle at 72° C. for 2 min. The PCR products were analyzed by gel electrophoresis.

F. mRNA Isolation and cDNA Preparation.

mRNA of DM1 myotubes was isolated with the Kingfisher Flex purification system (Thermal Fisher) in 96-well format following the manufacturer's instruction. cDNA was synthesized with SuperScript™ IV VILO™ Master Mix with ezDNase™ Enzyme (Thermal Fisher) following mRNA isolation.

G. ddPCR.

The primers and probes of ddPCR for hDMPK and GAPDH were pre-designed by Fisher Scientifics qPCR assays (Hs00266705_g1 and Hs01094329_m1). For DMPK expression, one target primer/probe set was used to detect DMPK expression levels, and one reference primer/probe set was used to detect GAPDH expression levels. The 24 μL of ddPCR reaction consisted of 12 μL of Supermix for Probes (no dUTP) (Bio-Rad Laboratories), 1 μL of Reference primers mix (21.6 μM), 1 μL of Reference probe (6 μM), 1 μL of Target primers mix (21.6 μM), 1 μL of Target probe (6 μM), 2 μL of H2O and 6 μL of sample genomic DNA. Droplets were generated using probe oil with the QX200 Droplet Generator (Bio-Rad Laboratories). Droplets were transferred to a 96-well PCR plate, sealed and cycled in a C1000 deep well Thermocycler (Bio-Rad Laboratories) under the following cycling protocol: 1 cycle at 95° C. for 10 min; 40 cycles of 94° C. for 30 sec, and 57° C. for 1 min; 1 cycle at 98° C. for 10 min (for enzyme inactivation). The cycled plate was then transferred and read in the FAM and HEX channels using the Bio-Rad QX200 Droplet Reader (Bio-Rad Laboratories). ddPCR analysis was performed with the Bio-Rad QuantaSoft Pro Software.

### H. FISH Staining of RNA Foci.

Primary myotubes were fixed for 15 min with 4% paraformaldehyde (PFA) and washed five times with 1×PBS for 5 min each at room temperature. Before staining, cells were permeabilized with 0.5% triton X-100 in 1×PBS for 5 min at room temperature, and then washed with 30% formamide and 2× saline-sodium citrate (SSC) mixture for 10 min at room temperature. Cells were then stained with 1 ng/μL of Cy3-PNA(CAG)₅ probe (PNA Bio, F5001) diluted in 30% formamide, 2×SSC, 2 μg/mL BSA, 66 μg/mL yeast tRNA, and 2 mM vanadyl complex for 15 min at 80° C. Following probe staining, cells were then washed in 30% formamide and 2×SSC mixture for 30 min at 60° C., then washed in 30% formamide and 2×SSC mixture for 30 min at 55° C., and then washed in 1×SSC solution for 10 min at room temperature, and finally washed in 1×PBS for 10 min at room temperature. For staining of myotube nuclei, cells were stained with anti-Myogenin antibody diluted in 1% bovine serum albumin (BSA) for overnight at 4° C. and washed twice with 1×PBS for 5 min each at room temperature. Cells were then incubated with the secondary antibody goat anti-rabbit Alexa 488 (Thermo Fisher, A32731) diluted in 1% BSA for 1 hr at room temperature and washed twice with 1×PBS for 5 min each at room temperature. Next, cells were stained with Hoechst solution (Thermo Fisher, H3569) at 0.1 mg/ml for 5 min and washed once with 1×PBS for 5 min. PBS was aspirated and fresh 100 μl of fresh PBS was added to each well. High-throughput acquisition of images was completed with the ImageXpress Micro Confocal High-Content Imaging System (Molecular Devices). RNA foci quantifications were accomplished with a customized analysis module of the MetaXpress program (Molecular Devices).

### 2. In Vitro Results

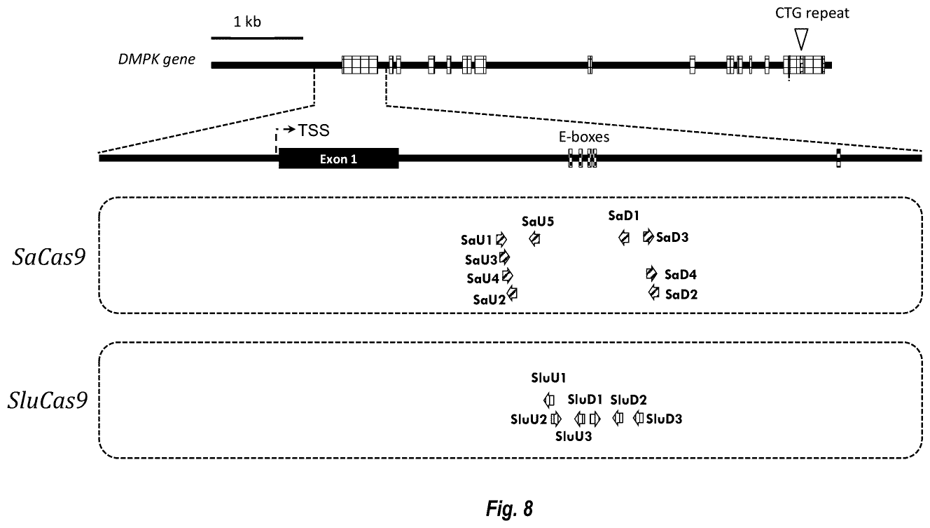

Nine (9) SaCas9 sgRNAs with the NNGRRT Protospacer Adjacent Motif (PAM) sequences were selected for targeting the four E-boxes within intron 1 of human DMPK gene; 5 of them (SaU1-SaU5) are located upstream of the four E-boxes and 4 of them (SaD1-SaD4) are located downstream of the four E-boxes (Table 5A and FIG. 8).

For SluCas9, a total of 97 sgRNAs with the NNGG PAM sequences were selected to target the four E-boxes region±150 bp region within intron 1 of the human DMPK gene (Table 5B). Three upstream sgRNAs (SluU1-SluU3) located upstream of the four E-boxes, and three downstream sgRNAs (SluD1-SluD3) located downstream of the four E-boxes were selected for use in the in vitro study (Table 5C and FIG. 8).

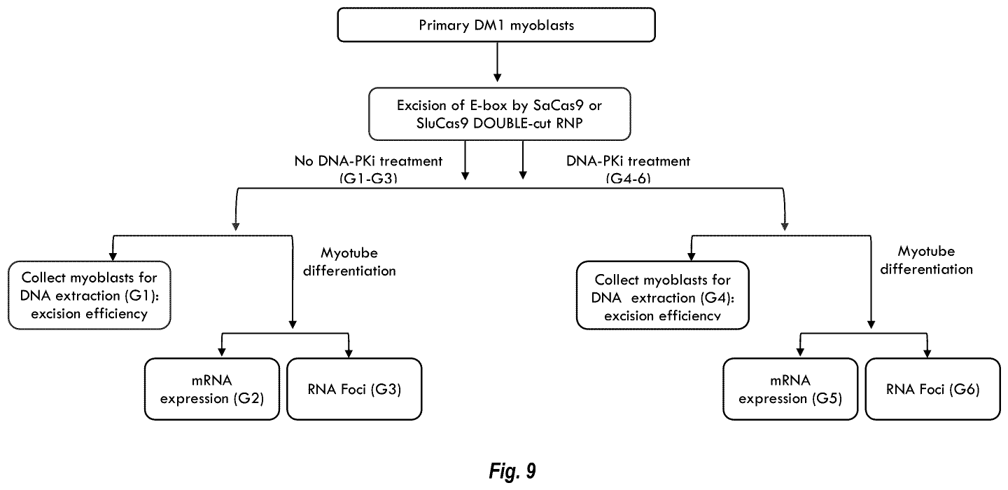

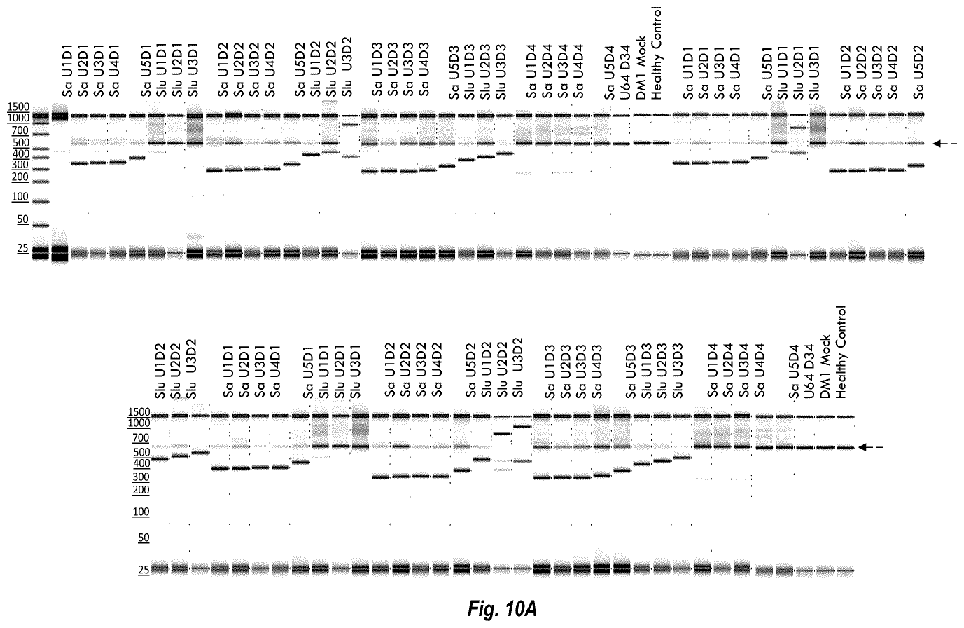

As a proof-of-concept (PoC), the 9 SaCas9 sgRNAs described above in Table 5A were paired, which resulted in total of 20 saCas9 sgRNA pairs (i.e., in Table 5A, where each upstream guide (designated with a “U”), was paired with each downstream guide (designated with a “D”) for a total of 20 total guide pairs). Six SluCas9 sgRNAs were also paired (Table 5C), which resulted in 9 combinations of SluCas9 sgRNA pairs (i.e., in Table 5C, where each upstream guide (designated with a “U”), was paired with each downstream guide (designated with a “D”) for a total of 9 total guide pairs). Each sgRNA pair consisting of one upstream sgRNA and one downstream sgRNA were assembled with recombinant SaCas9 or SluCas9 protein, respectively, into a ribonucleoprotein (RNP) complex and delivered into primary DM1 patient myoblasts with Amaxa 4D-Nucleofector. Post nucleofection, myoblasts were divided into 6 groups and were treated with DMSO (Groups 1-3) or 3 μM of DNA-dependent Protein Kinase Inhibitor (DNA-PKi) Compound 6 (Group 4-6) for 48 hrs. DNA-PKi was used to boost the excision efficiency and to better understand the contribution of E-boxes to DMPK transcription. 72 hrs post nucleofection, myoblasts from Groups 1 and 4 were collected for genomic DNA isolation, whereas the rest of the groups were subjected to myogenic differentiation to form myotubes. The myotubes were collected on Day 15 post nucleofection (Day 12 post differentiation) for mRNA isolation (Group 2 and 5) and RNA foci staining (Groups 3 and 6) (FIG. 9).

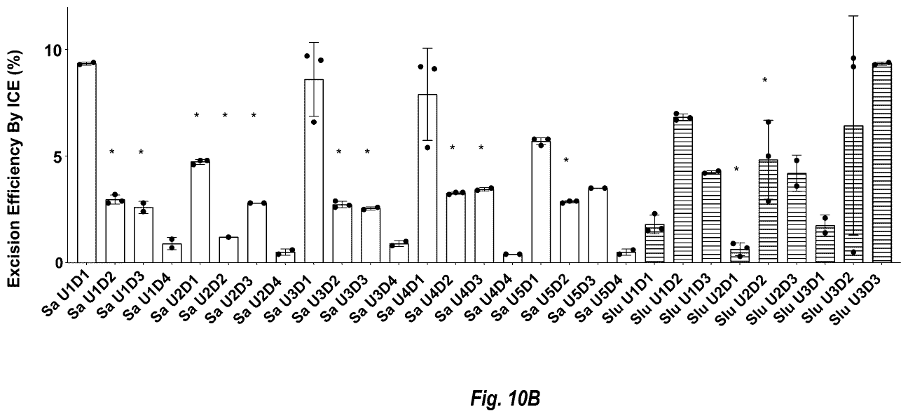

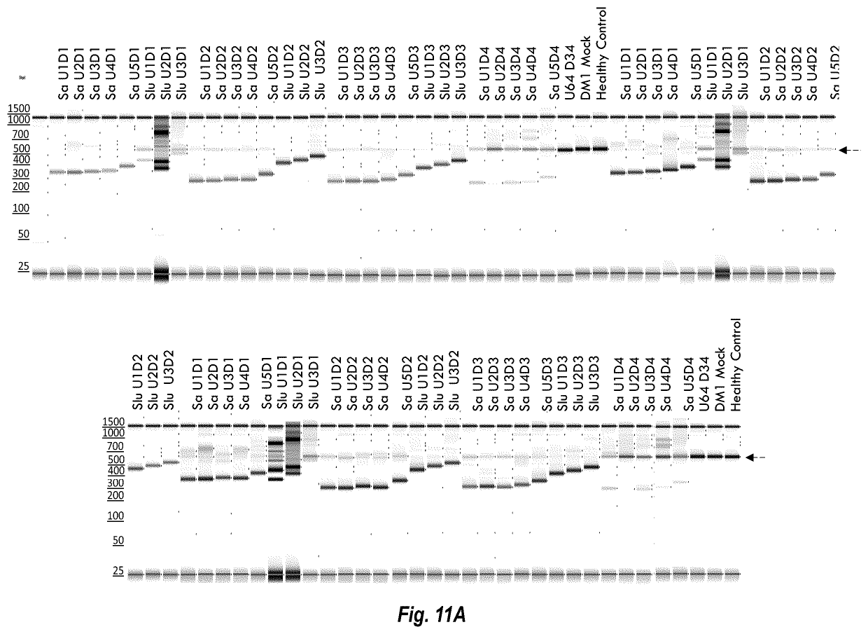

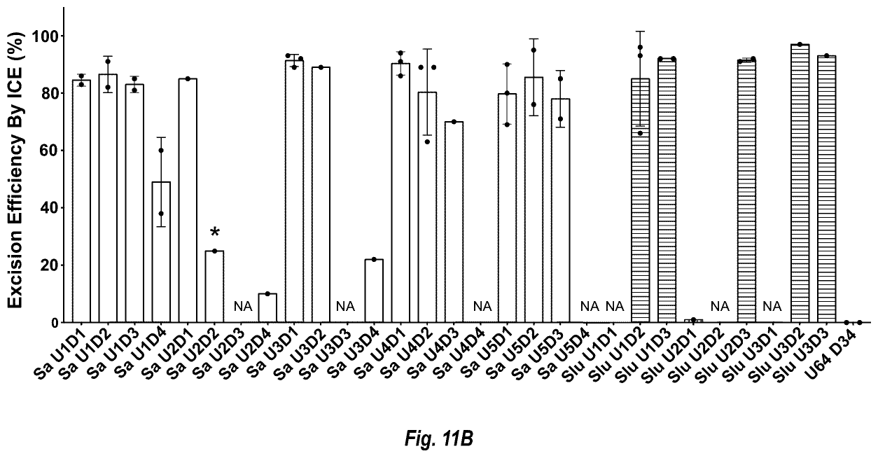

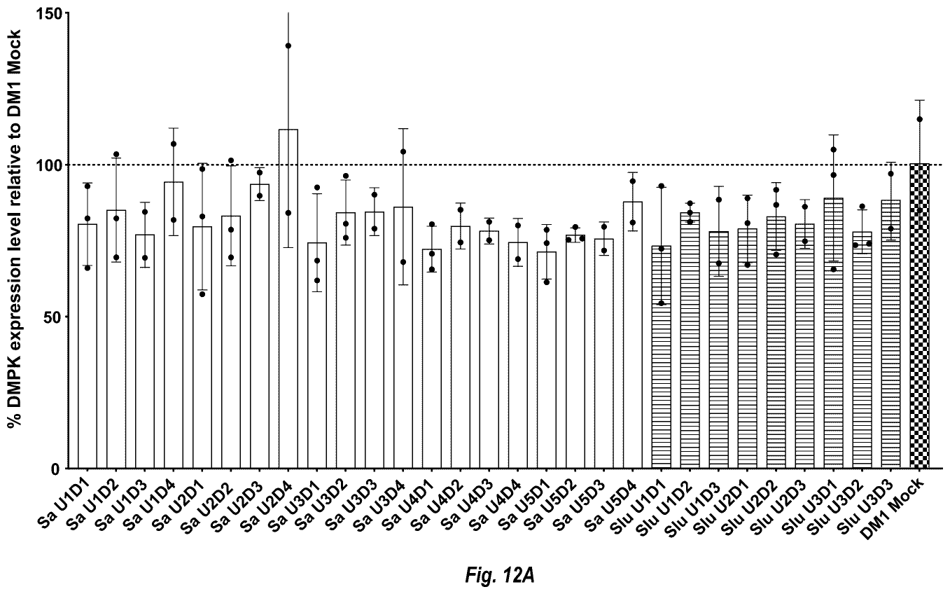

The ability of paired sgRNAs to induce excision of E-boxes was verified by PCR using genomic DNA prepared from Group 1 and 4 myoblast samples and a primer set flanking the E-box region (with anormal allele amplicon size of 567 bp). The E-box region was amplified by PCR and the products were run on TapeStation to confirm the expected excision; and submit for Sanger sequencing. TapeStation analysis was used to assess the excision profile induced by individual SluCas9 sgRNA pairs (FIG. 10A for vehicle group and FIG. 11A DNA-PKi group). Many SluCas9 sgRNA pairs induced excision of expected sizes which were represented by the PCR band located below the 567 bp PCR band. Compared to the vehicle group, the DNA-PKi group showed more apparent expected excision represented by the higher intensity of the smaller amplicon size band (FIG. 10A for vehicle group and FIG. 11A DNA-PKi group). The ICE analysis of excision efficiency confirmed as much (FIGS. 10B and 11B). Excision (Dual-cut) efficiency were estimated using Sanger sequencing results in the ICE (inference of CRISPR edits) analysis algorithm (https://ice.sherlock.vrtx.com/) (FIGS. 10B and 11B). ICE analysis is a method that quantifies the identity and prevalence of indels using Sanger sequencing data (Hsiau, T. et al. (2018) bioRxiv https://www.biorxiv.org/content/10.1101/251082v3).

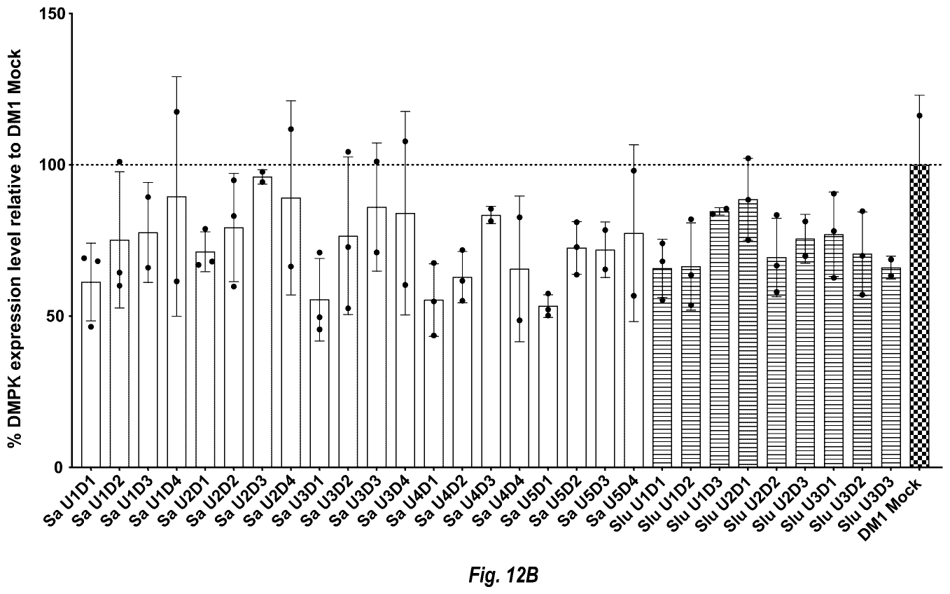

Next, the DMPK mRNA expression levels were assessed in DM1 myotubes (Group 2 and Group 5) through ddPCR. DM1 Mock samples were used as control to normalize the DMPK mRNA expression. Among the 29 pairs that are assayed by ddPCR DMPK expression assay, most of the pairs showed at least 20-30% of DMPK mRNA level in DMSO group (FIG. 12A). Consistent with the ICE analysis, DNA-Pki treatment group showed more dramatic hDMPK mRNA reduction with the top performing pairs reaching nearly 50% reduction (FIG. 12B).

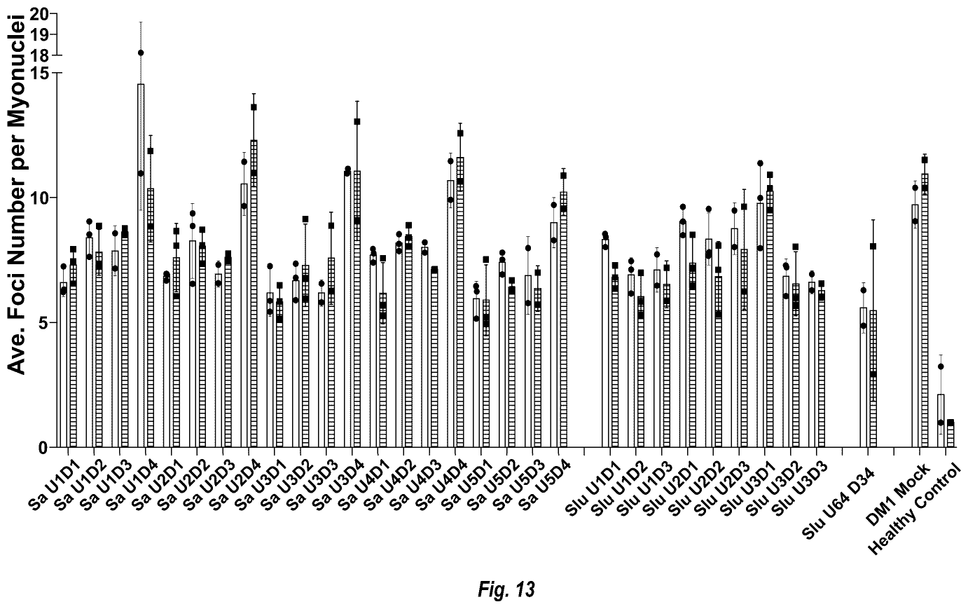

FISH staining of RNA foci showed reduction of CUG foci (formed by the CUG repeat expansion in the DMPK mRNA) in myonuclei of DM1 patient myotubes by individual sgRNA pairs with (open bar) or without (grey bar) DNA-PKi treatment (FIG. 13). Among the 20 SaCas9 sgRNA pairs and 9 SluCas9 sgRNA pairs evaluated, most of the SaCas9 and sluCas9 sgRNA pairs showed significant reduction of the average Foci number per myonuclei, indicating the reducing the CUG foci level in the differentiated myotubes. Several of the most efficient pairs (SaU1DI, SaU3D1, SaU3D3, saU5D1 sluU3D2, and sluU3D3; SEQ ID NOs: 1300/1305, 1302/1305, 1302/1307, 1304/1305, 1302/1306, and 1302/1307, respectively), showed similar levels of average Foci reduction (˜50%) as the most efficient CTG excision sluCas9 sgRNA pair (a control sluU64D34, Wherein SluU64 has a sequence of ggggcTCGAAGGGTCCTTGTAG (SEQ ID NO: 1497) and SluD34 has a sequence of CGCGCCAGACGCTCCCCAGAGC (SEQ ID NO: 1498)) in these DM1 patient myotubes (FIG. 13). Unlike the ICE analysis, no further average foci number reduction in myonuclei were observed in DNA-PKi group (FIG. 13 open (without DNA-PKi) versus gray bars (with DNA-PKi)).

### IX. Example 3: Single Clone Study

### 1. Materials and Methods

A. SpCas9 sgRNA Selection

A study of targeting excision of transcription start site (TSS) and four E-boxes and/or other cis-regulatory elements in the DMPK promoter and intronic enhancer region with spCas9 guide pairs was conducted. As described herein, the efficiency of DMPK mRNA level reduction both in myoblast and myotubes was demonstrated (FIGS. 5A-B), which also translated into a clear foci reduction and correction of mis-spliced gene in DM1 patient primary myotubes (FIGS. 6A-B). These results demonstrate the potential of targeting cis-element and TSS of human DMPK gene to reduce the toxic CUG repeat containing RNA transcript and therefore correct the mis-splicing caused by MBNL1 sequestration by those toxic RNA transcripts.

To further determine the ability of each cis-element region and TSS to mediate reduction of DMPK transcript, immortalized healthy myoblast was utilized to develop multiple clonal derivation myoblast lines bearing specific region deletion and assess their DMPK expression.

Provided herein are the data from clonal cell line derivation and evaluation of each cis-element region and TSS region ability to reduce the expression of DMPK mRNA transcripts.

### 2. Results

TABLE 6A
sgRNA for Clonal Cells

| SaCas9 | SEQ | | | Position | Position | |
|---|---|---|---|---|---|---|
| sgRNA | ID | | Protospacer sequence | on | in | PAM |
| name | NO | Strand | (20 nt) | Chr 19 | intron | sequence |
| Sp66 | 1500 | 1 | GGAGACAGGTCTGGGCACAG | 45782127 | 66 | AGG |
| Sp1182 | 1501 | −1 | GAGGCCAGGAGAGTCATTAG | 45781011 | 1182 | GGG |
| Sp2246 | 1502 | 1 | AGAGAAGGGGAGACAGACAG | 45779947 | 2246 | AGG |
| Sp424 | 1503 | 1 | AAGCCAGGAGGGCCAGCATG | 45781769 | 424 | GGG |
| Sp503 | 1504 | −1 | TCACCGCCCTCCCAGTGCCT | 45781690 | 503 | GGG |
| TSS-SpU2 | 1505 | 1 | TGTGAGGGGTTAAGGCTGGG | 45782565 | NA | AGG |

TABLE 6B
sgRNA Pairs for Single Clone

| Region of | | | | | |
|---|---|---|---|---|---|
| deletion | Region A | Region B | Region C | Region D | Region E |
| Down | Sp66 (SEQ | Sp1182 (SEQ | Sp2246 (SEQ | Sp503 (SEQ | sp2246 (SEQ |
| guides | ID NO: 1500) | ID NO: 1501) | ID NO: 1502) | ID NO: 1504) | ID NO: 1502) |
| Up | TSS-SpU2 | Sp66(SEQ | Sp1182 (SEQ | Sp424 (SEQ | sp66 (SEQ |
| guides | (SEQ ID | ID NO: 1500) | ID NO: 1501) | ID NO: 1503) | ID NO: 1500) |
| | NO: 1505) | | | | |

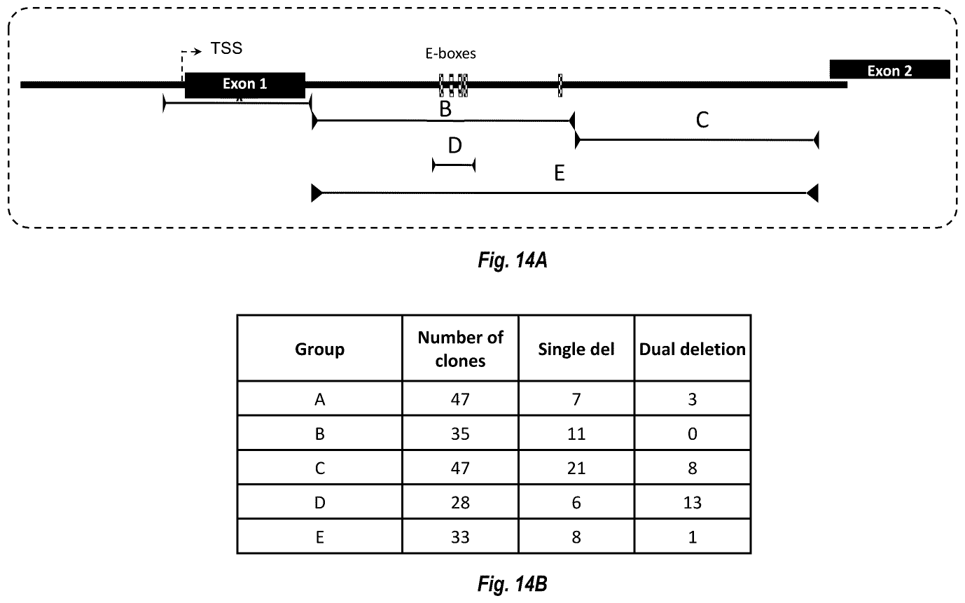

Six NGG Protospacer Adjacent Motif (PAM) SpCas9 sgRNAs were selected for targeting TSS and various region of cis-elements of human DMPK gene (Table 6A-B and FIG. 14A-B). A total of 5 regions (A, B, C, D, and E) were targeted with spCas9 guides to generate single-cell clonal derivation to evaluate each regions' ability in reduction of DMPK gene transcripts (FIG. 14A). In order to generate respective region of deletion, the sgRNA were paired according to Table 6B. Each SpCas9 sgRNA pair consisted of one upstream sgRNA and one downstream sgRNA and each sgRNA pair was assembled with recombinant SpCas9 protein to form a ribonucleoprotein (RNP) complex and delivered to immortalized healthy human myoblasts using the Amaxa 4D-Nucleofector. Post nucleofection, cells were cultured for 3 days and then single cells were sorted using Sony FACS machine into each individual well of a 96-well cell culture plate pre-coated with Matrigel and fresh myoblast culture medium. Clonal cell derived from single cells were allowed to expand in 96 well plate for 1-2 weeks and then split into two fraction one for cell banking and another fraction for genomic DNA isolation and genotyping for excision of the respective region. A total of 28 to 47 starting single-cell clones were obtained (FIG. 14B). Genotyping confirmed the successful bi-allelic excision of each respective region. 0-13 single cell derived clonal myoblast lines were obtained for each region (FIG. 14B).

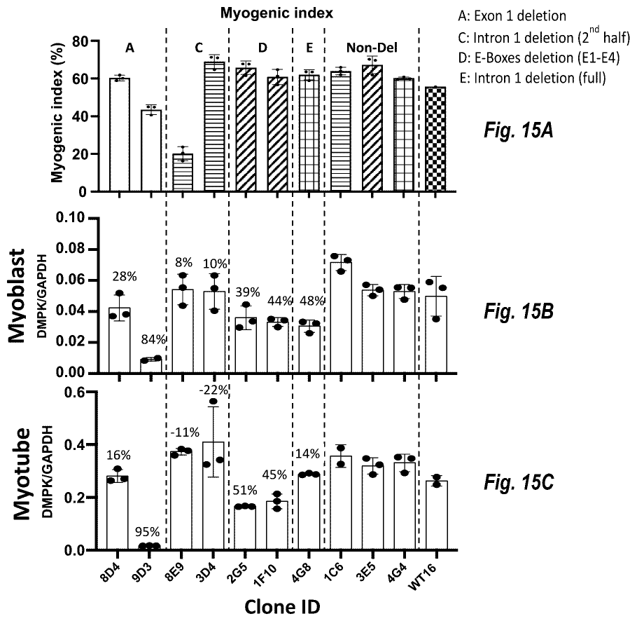

Next, DMPK mRNA expression levels were assessed in the clonal myoblasts derived from single cells both in myoblast stage and differentiated myotube stages using ddPCR (FIGS. 15A-B).

Of the sgRNA pairs targeting different regions (A to E) tested, all clonal cells with the E-box region excision and 1 clonal cell targeting TSS region showed the largest reduction of DMPK transcript levels in both myoblasts (FIG. 15B) and myotubes (FIG. 15C), indicating targeting TSS and E-box region has great potential to mediate DMPK mRNA reduction. Because interruption of TSS can result in total knockout of DMPK gene expression, and therefore may interfere with the normal function of DMPK gene in skeletal muscle and heart, the SaCas9 and SluCas9 study was focused on the Ebox region.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

1. A method of gene editing, comprising contacting a cell with
a. one or more guide RNAs or nucleic acids encoding one or more guide RNAs; and
b. an RNA-targeted endonuclease, or a nucleic acid encoding the RNA-targeted endonuclease,
wherein the guide RNA comprises a guide sequence that directs the RNA-targeted endonuclease to or near one or more regulatory elements that at least partially drive expression of a gene, and wherein the method causes disruption and/or excision of at least a portion of the one or more regulatory elements, wherein the gene is selected from: DM1 protein kinase (DMPK), CCHC-type zinc finger nucleic acid binding protein (CNBP), transcription Factor 4 (TCF4), junctophilin 3 (JPH3), huntingtin (HTT), ataxin 1 (ATXN1), ataxin 2 (ATXN2), ataxin 3 (ATXN3), ataxin 7 (ATXN7), ATXN8 opposite strand lncRNA (ATXN8OS), androgen receptor (AR), atrophin 1 (ATN1), fragile X Mental Retardation 1 (FMR1), chromosome 9 open reading frame 72 (C90RF72), polyadenylate-binding protein 2 (PABP2), amyloid precursor protein (APP), presenilin 1 (PSEN1), presenilin 2 (PSEN2), apolipoprotein E4 (APOE4), and synuclein alpha (SNCA).

2. (canceled)

3. A method of treating DM1 in a human subject, comprising administering
a. one or more guide RNAs or nucleic acids encoding one or more guide RNAs; and
b. an RNA-targeted endonuclease, or a nucleic acid encoding the RNA-targeted endonuclease,
wherein the guide RNA comprises a guide sequence that directs the RNA-targeted endonuclease to or near one or more regulatory elements that at least partially drive expression of the DMPK gene, and wherein the method causes disruption and/or excision of at least a portion of the one or more regulatory elements.

4. The method of claim 1, wherein the gene is associated with aberrant nucleotide repeats.

5. (canceled)

6. The method of claim 1, where the gene is DMPK.

7. The method of claim 1, wherein the one or more regulatory elements is a transcription start site (TSS), an E-Box, or a TSS and an E-Box.

8. (canceled)

9. (canceled)

10. The method of claim 1, wherein the gene is HTT or FMR1.

11. The method of claim 10, wherein
a. the gene is HTT and the one or more regulatory elements is selected from NF-κB binding domain, 20 bp direct repeats, transcription factor AP2, transcription factor Sp1, Huntington disease gene regulatory region-binding protein 1 and 2 (HDBP1 and HDBP2), and uncharacterized regulatory region (UCRR); or
b. the gene is FMR1 and the one or more regulatory elements is selected from 22-bp methylation sensitive element (MSE) and the single-nucleotide polymorphism (SNP) variant comprising a T/C at ss71651738.

12.-30. (canceled)

31. The method of claim 1, wherein
a. the guide sequence directs the RNA-targeted endonuclease to a target sequence within 100, 200, 300, 400, 500, 600, 700, 800, 900, 1000, 1100, 1200, 1300, 1400 or 1500 nucleotides of a regulatory element;
b. a pair of guide RNAs comprising a pair of guide sequences directs the RNA-targeted endonuclease to two target sequences flanking a regulatory element, wherein at least one or both of the target sequences flanking the regulatory element are within 5, within 10, within 20, within 30, within 40, within 50 or within 100 nucleotides of the regulatory element; or
c. a pair of guide RNAs excises at least a portion of a regulatory element, wherein excised portion is less than 3000, 2500, 2200, 2000, 1800, 1500, 1400, 1300, 1200, 1100, 1000, 900, 800, 700, 600, 500, 400, 300, 200, 100, 50, or 25 nucleotides in length.

32.-40. (canceled)

41. The method of claim 1, wherein the regulatory element is an enhancer or a promoter.

42. (canceled)

43. The method of claim 1, wherein the RNA-targeted endonuclease is a Cas nuclease, optionally wherein the Cas nuclease is Cas9 or is a Cpf1 nuclease.

44. (canceled)

45. The method of claim 43, wherein the Cas9 nuclease is from Streptococcus pyogenes (spCas9), Staphylococcus aureus (saCas9), or Staphylococcus lugdunensis (sluCas9).

46.-58. (canceled)

59. The method of claim 1, wherein the guide RNA is associated with a lipid nanoparticle (LNP), lipoplex, or a viral vector.

60.-82. (canceled)

83. A composition comprising a first guide RNA and a second guide RNA, wherein the first guide RNA directs an RNA-targeted endonuclease to or near the 3′ end of a regulatory element that at least partially drives expression of a gene that produces a toxic protein and/or toxic transcript, and wherein the second guide RNA directs a RNA-targeted endonuclease to or near the 5′ end the regulatory element.

84. The composition of claim 83, wherein the regulatory element is in an intron, a transcription start (TSS) site, an intronic E-box site, or is in exon 1 of the DMPK gene.

85.-88. (canceled)

89. The composition of claim 83, wherein the gene is HTT and the regulatory element is a cis-regulatory element for regulating gene expression, or the gene is FMR1 and the regulatory element is a 22-bp methylation sensitive element (MSE).

90. (canceled)

91. (canceled)

92. The composition of claim 83, wherein first and second guide RNAs function to excise at least a portion of a regulatory element, wherein the excised portion is less than 3000, 2500, 2200, 2000, 1800, 1500, 1400, 1300, 1200, 1100, 1000, 900, 800, 700, 600, 500, 400, 300, 200, 100, 50, or 25 nucleotides in length.

93-95. (canceled)

96. The composition of claim 83, wherein the guide RNA is associated with a lipid nanoparticle (LNP), lipoplex, or one or more viral vectors.

97. (canceled)

98. The composition of claim 96 comprising
a. more than one viral vector, wherein a first vector comprises one or more guide RNAs and a second vector comprises a nucleic acid encoding an RNA-targeted endonuclease, optionally wherein the RNA-targeted endonuclease is a Cas nuclease or a Cpf1 nuclease; or
b. one viral vector comprising at least one guide RNA and a nucleic acid encoding an RNA-targeted endonuclease, optionally wherein the RNA-targeted endonuclease is a Cas nuclease or a Cpf1 nuclease.

99. (canceled)

100. (canceled)

101. The composition of claim 98, wherein the Cas nuclease is Cas9, optionally wherein the Cas9 nuclease is from Streptococcus pyogenes (spCas9), Staphylococcus aureus (saCas9), or Staphylococcus lugdunensis (sluCas9).

102.-107. (canceled)

108. The composition of claim 83, wherein
a. the first guide RNA comprises at least one guide sequence selected from:
i. at least 17, 18, 19, or 20 contiguous nucleotides of any one of SEQ ID NOs: 1, 2, or 3;
ii. at least 17, 18, 19, or 20 contiguous nucleotides of a sequence complementary to any one of SEQ ID NOs: 1, 2, or 3; or
iii. a sequence that binds to a sequence that is complementary to any one of SEQ ID NOs: 1, 2 or 3; and
the second guide RNA comprises at least one guide sequence selected from:
i. at least 17, 18, 19, or 20 contiguous nucleotides of any one of SEQ ID NOs: 4, 5, or 6;
ii. at least 17, 18, 19, or 20 contiguous nucleotides of a sequence complementary to any one of SEQ ID NOs: 4, 5, or 6; or
iii. a sequence that binds to a sequence that is complementary to any one of SEQ ID Nos: 4, 5 or 6; or
b. the first guide RNA comprises at least one guide sequence selected from:
i. at least 17, 18, 19, or 20 contiguous nucleotides of any one of SEQ ID NOs: 7, 8, or 9;
ii. at least 17, 18, 19, or 20 contiguous nucleotides of a sequence complementary to any one of SEQ ID NOs: 7, 8, or 9; or
ii. a sequence that binds to a sequence that is complementary to any one of SEQ ID NOs: 7, 8 or 9; and
the second guide RNA comprises at least one guide sequence selected from:
i. at least 17, 18, 19, or 20 contiguous nucleotides of any one of SEQ ID NOs: 10, 11, or 12;
ii. at least 17, 18, 19, or 20 contiguous nucleotides of a sequence complementary to any one of SEQ ID NOs: 10, 11, or 12: or
ii. a sequence that binds to a sequence that is complementary to any one of SEQ ID NOs: 10, 11 or 12:
c the first guide RNA comprises at least one guide sequence selected from:
i. at least 17, 18, 19, 20, 21, or 22 contiguous nucleotides of any one of SEQ ID NOs: 1300, 1301, 1302, 1303, or 1304;
ii. at least 17, 18, 19, 20, 21, or 22 contiguous nucleotides of a sequence complementary to any one of SEQ ID NOs: 1300, 1301, 1302, 1303, or 1304; or
iii. a sequence that binds to a sequence that is complementary to any one of SEQ ID NOs: 1300, 1301, 1302, 1303, or 1304; and
the second guide RNA comprises at least one guide sequence selected from:
i. at least 17, 18, 19, 20, 21, or 22 contiguous nucleotides of any one of SEQ ID NOs: 1305, 1306, 1307, or 1308;
ii. at least 17, 18, 19, 20, 21, or 22 contiguous nucleotides of a sequence complementary to any one of SEQ ID NOs: 1305, 1306, 1307, or 1308: or
iii. a sequence that binds to a sequence that is complementary to any one of SEQ ID Nos: 1305, 1306, 1307, or 1308: or
d. the first guide RNA comprises at least one guide sequence selected from:
i. at least 17, 18, 19, 20, 21, or 22 contiguous nucleotides of any one of SEQ ID NOs: 1420, 1428, or 1475;
ii. at least 17, 18, 19, 20, 21, or 22 contiguous nucleotides of a sequence complementary to any one of SEQ ID NOs: 1420, 1428, or 1475: or
iii. a sequence that binds to a sequence that is complementary to any one of SEQ ID NOs: 1420, 1428, or 1475; and
the second guide RNA comprises at least one guide sequence selected from:
i. at least 17, 18, 19, 20, 21, or 22 contiguous nucleotides of any one of SEQ ID NOs: 1402, 1411, or 1463;
ii. at least 17, 18, 19, 20, 21, or 22 contiguous nucleotides of a sequence complementary to any one of SEQ ID NOs: 1402, 1411, or 1463: or
ii. a sequence that binds to a sequence that is complementary to any one of SEQ ID NOs: 1402, 1411, or 1463.

109.-126. (canceled)
